(19) United States
(12) Patent Application Publication
(10) Pub. No.: US 2023/0114479 A1
(43) Pub. Date: Apr. 13, 2023

(54) WEARABLE DEVICE AND COMPUTATIONAL PLATFORM FOR DETECTING AND DETERMINING CANDIDATE TREATMENTS FOR A BIOLOGICAL CONDITION

(71) Applicant: Moonshot Wearables, Inc., Cary, NC (US)

(72) Inventors: Chris Nyers, Cary, NC (US); James L. Sword, Garland, TX (US); Subir Mukherjee, Wake Forest, NC (US)

(21) Appl. No.: 17/802,730

(22) PCT Filed: Feb. 26, 2021
(86) PCT No.: PCT/US2021/019839

§ 371 Date: Aug. 26, 2022

Related U.S. Application Data
(60) Provisional application No. 62/981,661, filed on Feb. 26, 2020.

(51) Int. Cl. A61B 5/145; A61B 5/00; A61B 5/1455

(52) U.S. Cl. CPC A61B 5/14546; A61B 5/7275; A61B 5/14551; A61B 5/6838; A61B 5/6826

(57) ABSTRACT

Apparatuses, systems, and techniques are described to detect one or more biological conditions. In one or more implementations, the presence of anemia can be detected in individuals. A wearable device can collect data regarding amounts of attenuation of electromagnetic radiation having one or more wavelength ranges. The amounts of attenuation of the electromagnetic radiation can be used to determine measures of biomolecules and the measures of biomolecules can be used to determine probabilities of individuals being diagnosed with anemia. Additionally, one or more implementations of a computational platform can facilitate the exchange of information between healthcare practitioners and patients regarding the diagnosis and treatment of one or more biological conditions. The computational platform can also analyze large amounts of information to generate computational models to determine probabilities of individuals being diagnosed with anemia and to determine candidate treatments for anemia.

### CLAIM OF PRIORITY

This patent application claims the benefit of priority to U.S. Provisional Application Ser. No. 62/981,661, filed Feb. 26, 2020, which is incorporated by reference herein in its entirety.

### BACKGROUND

Anemia is a deficiency in red blood cells or hemoglobin, both of which are necessary to transport oxygen to sustain life. According to the World Health Organization, 25% of the world's population is affected by anemia. The World Health Assembly and UNICEF have identified anemia as a “global health crisis,” and selected it as one of six global targets for improving maternal, infant, and young children's nutrition by 2025. Anemia impacts the global economy (4.05% of worldwide GDP=$3T annually). The illness creates a greater incidence of maternal death and miscarriage (avg. of 22%), a decrease in cognition, and negatively impacts day-to-day quality of life. Anemia can often be present in children and pregnant women. Anemia can be particularly prevalent in the developing world. Anemia, the most common blood disorder is not one illness. There are different types of anemia: aplastic, iron deficiency, sickle cell, thalassemia, vitamin deficiency, and hemolytic.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example environment to obtain data used in detecting and determining candidate treatments for one or more biological conditions or issues.

FIG. 2 illustrates an example wearable device that includes sensor circuitry to detect levels of hemoglobin metalloproteins and to determine hematocrit levels present in the blood of individuals.

FIG. 3 illustrates an example wearable device that can be placed on a finger and that includes an array of emitters that each transmit electromagnetic radiation at different wavelengths and one or more detectors that detect electromagnetic radiation produced by the array of emitters.

FIG. 4 illustrates a side view of an example wearable device that generates data that can be used to determine a probability of anemia being present in an individual.

FIG. 5A illustrates a top view of an example wearable device that generates data that can be used to determine a probability of anemia being present in an individual.

FIG. 5B illustrates a bottom view of an example wearable device that generates data that can be used to determine a probability of anemia being present in an individual.

FIG. 6 illustrates another example wearable device that includes sensor circuitry to detect levels of hemoglobin metalloproteins and to determine hematocrit levels present in the blood of individuals with a clamp member to attach to a finger of an individual.

FIG. 7 illustrates another example wearable device that can be placed on a finger and that includes an array of emitters that each transmit electromagnetic radiation at different wavelengths and one or more detectors that detect electromagnetic radiation produced by the array of emitters with the emitters and detectors positioned on a same side of the wearable device.

FIG. 8A illustrates a top view of an example of an additional wearable device that generates data that can be used to determine a probability of anemia being present in an individual.

FIG. 8B illustrates a side view of an example of an additional wearable device that generates data that can be used to determine a probability of anemia being present in an individual.

FIG. 9 illustrates an example environment to facilitate communication between individuals, healthcare practitioners, such as doctors, nurses, and health support workers, regarding information corresponding to the presence of one or more biological conditions or issues.

FIG. 10 illustrates an example environment to obtain and analyze information related to the detection, diagnosis, and candidate treatments of one or more biological conditions or issues.

FIG. 11 illustrates an example framework to generate one or more computational models that can determine a probability that an individual is anemic, a type of anemia associated with the individual, and/or determine one or more candidate treatments for anemia.

FIG. 12 is a flow diagram of an example process for a standalone wearable device to use sensor data to provide an indication of a probability of anemia being present in an individual.

FIG. 13 is a flow diagram of an example process to analyze electromagnetic emission and detection information to determine a probability of anemia being present in an individual.

FIG. 14 is a flow diagram of an example process for a system to analyze at least sensor data to determine a probability of anemia being present in an individual and to determine one or more candidate treatments for the individual with respect to anemia.

FIG. 15 is a block diagram illustrating an example architecture of software, which can be installed on any one or more of the devices described herein.

FIG. 16 illustrates a diagrammatic representation of a machine in the form of a computer system within which a set of instructions may be executed for causing the machine to perform any one or more of the methodologies or processes discussed herein, according to one or more example implementations.

### DETAILED DESCRIPTION

Conventional techniques for the detection of anemia are typically invasive and require obtaining blood samples from individuals because physical examination of patients with anemia can indicate symptoms that may be related to other biological conditions. Specialized devices and technicians are often used to obtain blood samples from individuals and to operate the devices that analyze the blood samples to identify individuals that are anemic. However, anemia is most prevalent in the developing parts of the world and in rural areas where resources are typically low. Thus, the equipment used to detect anemia and the workers needed to obtain blood samples and operate the equipment are often unavailable in the locations where anemia is prevalent. Furthermore, conventional non-invasive devices and techniques that are used in the detection of anemia are inaccurate and/or imprecise in the results provided. In addition, conventional non-invasive techniques used in the detection of anemia also require equipment and/or technicians that are not readily available in environments with relatively low resources. Consequently, anemia often goes undetected and untreated in millions of individuals worldwide.

One or implementations described herein can be directed to a product ecosystem that includes a wearable device, a mobile device application, a handheld device, in some scenarios, and a computational platform. The product ecosystem can address various aspects related to one or more biological conditions. For example, one or more implementations herein can be directed to the detection of a biological condition. In addition, one or more implementations described herein can be related to generating diagnostic health information that can be used as part of the process implemented by healthcare practitioners to diagnose individuals with a biological condition. Further, one or more implementations described herein can be directed to determining suggested clinical treatments that can include recommendations for candidate treatments of a biological condition.

The wearable device can enable remote detection of a biological condition and monitoring of characteristics of individuals to determine real-time or near real-time intervention in relation to the biological condition. Additionally, a mobile device application can provide data visualization and insights to inform patients and/or healthcare practitioners with regard to real-time or near real-time decisions in relation to the biological condition. The mobile device application can also be used in telemedicine sessions between a patient and a healthcare practitioner. Accordingly, problems that can arise due to relatively large distances between patients and healthcare practitioners can be minimized and the cost of providing assistance to individuals that are diagnosed with the biological condition can be reduced.

Further, the computational platform can analyze data obtained from a number of data sources to identify insights into the health of individuals and to determine candidate treatments for a biological condition using one or more machine learning techniques. In various examples, the computational platform can be integrated with third-party platforms such that the computational platform can be extensible. The computational platform can also store electronic versions of medical records of individuals and, in some implementations, access electronic versions of medical records of individuals stored by third-party systems. In one or more examples, the computational platform can also provide access to information that can be used to educate and train healthcare practitioners and/or individuals in relation to a biological condition.

In illustrative examples, the implementations described herein are directed to one or more wearable devices that can be used, for example, to detect characteristics of blood of individuals to determine a probability that anemia is present in the individuals in a non-invasive manner. Additionally, the implementations described herein are directed to one or more systems to analyze patient information to determine one or more candidate treatments for anemia. Further, the implementations described herein can make anemia related information readily available to patients and healthcare providers.

In various implementations, a wearable device can be placed on or over a finger of an individual. In one or more implementations, the wearable device can be wrapped around a finger of an individual. For example, the wearable device can be a cap or have a tube-like structure that can be disposed over a finger of an individual. The wearable device can include emitter circuitry to emit electromagnetic radiation at a number of different wavelengths. The wearable device can also include detector circuitry to detect at least a portion of the electromagnetic radiation emitted by the emitter circuitry. Based on the amount of attenuation of the electromagnetic radiation emitted by the wearable device, blood-related information can be determined. The amount of attenuation of the electromagnetic radiation can include an amount of the electromagnetic radiation that is absorbed by one or more substances and an amount of the electromagnetic radiation that is scattered. For example, an amount of oxygenated hemoglobin can be determined based on the attenuation of one or more wavelengths of electromagnetic radiation emitted by the wearable device. In addition, an amount of deoxygenated hemoglobin can be determined based on the attenuation of one or more additional wavelengths of electromagnetic radiation emitted by the wearable device. The amount of attenuation of one or more wavelengths of electromagnetic radiation emitted by the wearable device can be used to determine packed cell volume (PCV). The packed cell volume can correspond to a volume percentage of red blood cells. Packed cell volume can also be referred to as hematocrit, volume of packed red cells, or erythrocyte volume fraction.

The wearable device can determine a measure of a probability of anemia being present in an individual at a given time based on the amount of at least one of oxygenated hemoglobin, deoxygenated hemoglobin, or packed cell volume. Additionally, the wearable device can include a plurality of indicators that each correspond to different levels of anemia or different probabilities of a diagnosis of anemia. For example, the wearable device can include a first indicator that corresponds to a first level of anemia or a first range of probabilities of a diagnosis of anemia, a second indicator that corresponds to a second level of anemia or a second range of probabilities of a diagnosis of anemia, and a third indicator that corresponds to a third level of anemia or a third range of probabilities of a diagnosis of anemia. The first indicator can correspond to measures of at least one of oxygenated hemoglobin, deoxygenated hemoglobin, or packed cell volume that are included in a first range of levels, the second indicator can correspond to measures of at least one of oxygenated hemoglobin, deoxygenated hemoglobin, or packed cell volume that are included in a second range of levels, and the third indictor can correspond to measures of at least one of oxygenated hemoglobin, deoxygenated hemoglobin, or packed cell volume that are included in a third range of levels. In various implementations, the first indicator can be associated with a first color, the second indicator can be associated with a second color, and the third indicator can be associated with a third color. In one or more illustrative examples, the first indicator can be a red color, the second indictor can be a yellow color, and the third indicator can be a green color.

The wearable device can also include one or more energy storage devices to provide energy for the wearable device to perform one or more functions. Further, the wearable device can include communications circuitry to wirelessly transmit and receive signals. The communications circuitry can be used to transmit blood-related information obtained by the wearable device to one or more additional devices. To illustrate, the communications circuitry can transmit blood-related information obtained by the wearable device to a mobile device of an individual that has placed the wearable device on a finger. The mobile device can execute an application to display the blood-related information received from the wearable device. In additional implementations, the communications circuitry can transmit blood-related information to a computing device or platform of a healthcare practitioner. In this way, the healthcare practitioner can access the blood-related information of the individual that is obtained from the wearable device.

The implementations of a wearable device described herein are different from conventional devices that may obtain some blood-related information because the conventional devices are typically data gathering devices that do not include features that enable the determination and indication of a level of anemia of an individual that is wearing the conventional devices. In addition, the devices that are performing computations to determine a probability of anemia being present in an individual have a larger form factor than the implementations of the wearable device described herein. Further, conventional devices that gather blood-related information typically have a separate communications module that is detached from a data gathering component. However, the implementations of a wearable device described herein include communications circuitry within the wearable device itself. Thus, the computational techniques, as well as the design and electronic components included in the implementations of the wearable device described herein provide functionality that is not typically found in conventional devices that gather blood-related information. Accordingly, the implementations of the wearable device described herein can operate as a standalone device that can be placed or worn on a finger of an individual that gathers blood-related information, analyzes the blood-related information to provide an indication of a probability of anemia being present in an individual, and wirelessly communicates the blood-related information gathered by the wearable device to additional devices.

Furthermore, implementations of systems are described herein that can obtain and store blood-related information from a number of individuals, such as hundreds of individuals, thousands of individuals, up to tens of thousands of individuals. The blood-related information can be analyzed according to one or more machine-learning techniques. In implementations, the blood-related information gathered by the one or more systems described herein can be analyzed to determine candidate treatments for anemia. The one or more systems can determine candidate treatments that can be effective for individuals that have one or more characteristics, such as genetic characteristics, physiological characteristics, and/or blood-related characteristics. The blood-related information can also be analyzed to identify individuals that may be in need of additional testing. For example, the implementations of one or more systems described herein can analyze blood-related information of one or more individuals to identify individuals that have at least a threshold probability of having an underlying biological condition that is causing anemia. The underlying biological condition can be genetically related. Additionally, the underlying biological condition can be a chronic condition or a disease, such as cancer. Further, the implementations of one or more systems described herein can analyze blood-related information to identify trends in anemic conditions of individuals and/or determine hotspots where anemia is present above a threshold amount. The implementations of one or more systems described herein can also enable healthcare practitioners and patients to access medical records of patients that can include blood-related information of patients, levels of anemia of patients, treatments of anemia provided to patients, and improvement in anemia-related conditions based on treatments received by patients. Healthcare practitioners and/or individuals can also have access to medical literature and research via the one or more systems described herein, such as anemia-related medical literature and research.

The systems described herein are different from conventional systems that simply monitor blood-related information of individuals. In particular, the systems described herein can aggregate blood-related information from many individuals and analyze the blood-related information using one or more machine learning techniques. The analysis of the blood-related information can be used to identify suggested clinical treatments for individuals that may be effective and to identify one or more biological characteristics of individuals that have a particular type of anemia. For example, the systems described herein can be used to identify genetic characteristics and/or morphological characteristics of individuals that have anemia. Implementations of systems described herein can also analyze blood-related information of a number of individuals to determine one or more suggested clinical treatments that can result in improvement of the anemic conditions of the number of individuals and to determine one or more suggested clinical treatments that are less effective in improving the anemic conditions of the individuals. In this way, information can be accessible to individuals and healthcare practitioners that was not previously available to aid in the diagnosis and treatment of anemia.

Further, the use of computing device applications and communication channels can facilitate communication between healthcare practitioners and patients separated by distance, as well as communication between various healthcare practitioners separated by distance, to aid in the diagnosis of anemia in individuals and the delivery of candidate treatments to individuals diagnosed with anemia. The computing device applications can also be used to provide targeted communications, alerts, and/or reminders to individuals or groups of individuals with respect to anemia. As a result, the ease of access to information related to at least one of the causes, symptoms, and/or suggested clinical treatments of anemia can increase.

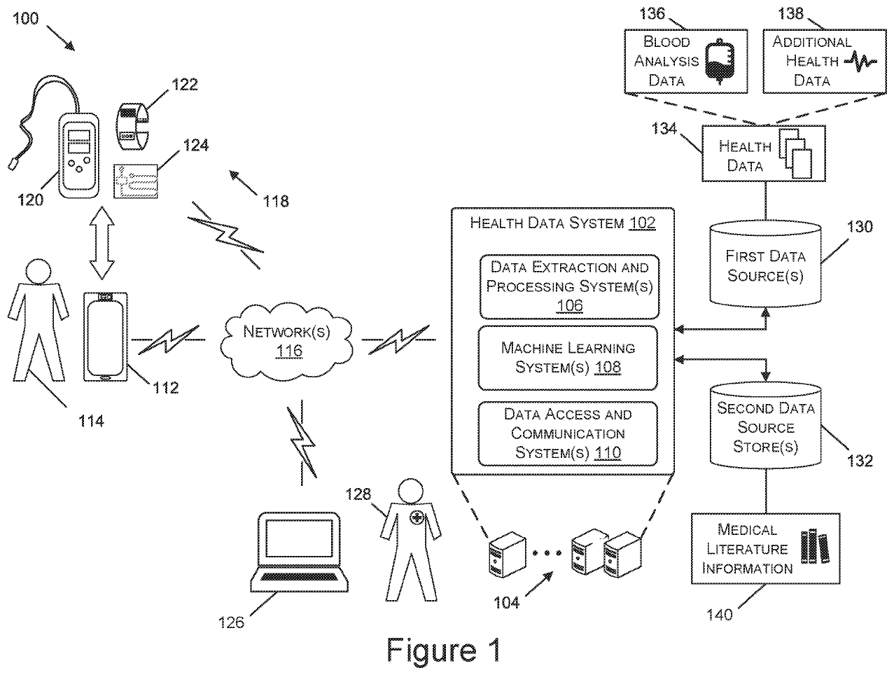

FIG. 1 illustrates an example environment to obtain data used in the detection, diagnosis, and determining candidate treatments of one or more biological conditions or issues. As used herein, a biological condition can refer to an abnormality of function and/or structure in an individual to such a degree as to produce or threaten to produce a detectable feature of the abnormality. A biological condition can be characterized by external and/or internal characteristics, signs, and/or symptoms that indicate a deviation from a biological norm in one or more populations. A biological condition can include at least one of one or more diseases, one or more disorders, one or more injuries, one or more syndromes, one or more disabilities, one or more infections, one or more isolated symptoms, or other atypical variations of biological structure and/or function of individuals. In some instances, described herein, a biological condition can be referred to as a “biological issue.” Additionally, a treatment, as used herein, can refer to a substance, procedure, routine, device, and/or other intervention that can administered or performed with the intent of alleviating one or more effects of a biological condition in an individual.

The environment 100 can include a health data system 102. The health data system 102 can obtain red blood cell information from several sources and make the red blood cell information accessible to individuals and healthcare providers. Additionally, the health data system 102 can analyze red blood cell information to determine a level of anemia for individuals. The health data system 102 can also analyze red blood cell information to determine one or more recommended treatments for individuals that are diagnosed with anemia. Additionally, the health data system 102 can make red blood cell information and anemia treatment information accessible to individuals and to healthcare practitioners through a centralized system. In various implementations, an application executed by computing devices of individuals and/or computing devices of healthcare practitioners can access the health data system 102 to obtain red blood cell information for individuals for a given period of time, obtain treatment protocols for anemia, retrieve information indicating whether or not anemia treatment protocols have been followed, and so forth.

The health data system 102 can be implemented by one or more computing devices 104. The one or more computing devices 104 can include one or more server computing devices, one or more desktop computing devices, one or more laptop computing devices, one or more tablet computing devices, one or more mobile computing devices, or combinations thereof. In certain implementations, at least a portion of the one or more computing devices 104 can be implemented in a distributed computing environment. For example, at least a portion of the one or more computing devices 104 can be implemented in a cloud computing architecture.

The health data system 102 can include one or more data extraction and processing systems 106. The one or more data extraction and processing systems 106 can obtain data from a number of data sources. The data obtained by the one or more data extraction and processing systems 106 can be received from one or more computing devices. In addition, the data obtained by the one or more data extraction and processing systems 106 can be extracted from one or more websites. Further, the data obtained by the one or more data extraction and processing systems 106 can be retrieved from one or more data stores. At least a portion of the one or more data stores can be located remotely from the health data system 102. In various implementations, the one or more data stores can be maintained by third parties that are different from the one or more entities that implement the health data system 102.

The health data system 102 can also include one or more machine learning systems 108. The one or more machine learning systems 108 can implement one or more machine learning techniques to analyze blood-related information and other health-related information to determine a level of anemia for individuals or a probability of a diagnosis of anemia for individuals. The one or more machine learning systems 108 can also determine one or more treatments for individuals based on the level of anemia associated with the individuals. In one or more illustrative examples, the one or more machine learning systems 108 can implement one or more multilayer perceptron networks. In additional illustrative examples, the one or more machine learning systems 108 can implement one or more Bayesian networks.

Additionally, the health data system 102 can include one or more data access and communication systems 110. The one or more data access and communication systems 110 can facilitate the access of information stored by and/or information calculated by the health data system 102. For example, the one or more data access and communication systems 110 can generate one or more user interfaces that individuals can access that show health data of the individuals, such as red blood cell information, blood oxygen levels, heart rate information, combinations thereof, and the like. The one or more data access and communication systems 110 can also generate one or more user interfaces that show health data for individual patients of a healthcare practitioner. Further, the one or more data access and communication systems 110 can generate one or more user interfaces that show aggregated health data for a number of patients of a healthcare practitioner. In various implementations, the one or more data access and communication systems 110 can exchange information with an application that is executed by computing devices of individuals and that is executed by computing devices of healthcare practitioners. To illustrate, the one or more data access and communication systems 110 can be in communication with a mobile device application that is executed by a mobile device of an individual and/or a mobile device of a healthcare practitioner. In addition, the one or more data access and communication systems 110 can send notifications to individuals and to healthcare practitioners. The notifications can indicate at least one of a level of anemia of an individual, a probability of a diagnosis of anemia for an individual, health information of individuals, red blood cell information of individuals, or anemia treatment information of individuals.

The environment 100 can also include a computing device 112 that is operated by an individual 114. The computing device 112 can include a mobile computing device, a smart phone, a tablet computing device, a laptop computing device, a desktop computing device, one or more combinations thereof, and the like. The computing device 112 can send blood-related information and/or other health information of the individual 114 to the health data system 102. The computing device 112 can also access health information of the individual 114 via the health data system 102. In various implementations, the computing device 112 can execute an application to communicate with the health data system 102. The individual 114 can be an individual that has been previously diagnosed with anemia. For example, the individual 114 can have previous blood-related information resulting in the individual 114 being diagnosed with anemia. In additional examples, the individual 114 can undergo initial testing for anemia or the individual 114 may have been previously tested for anemia with no prior diagnosis of anemia.

The computing device 112 can transmit and receive data via one or more networks 116. The one or more networks 116 can be representative of any one or combination of multiple different types of wired and wireless networks, such as the Internet, cable networks, cellular networks, satellite networks, wide area wireless communication networks, wireless local area networks, wired local area networks, and public switched telephone networks (PSTN).

Blood-related information of the individual 114 can be obtained by one or more data capture devices 118. The one or more data capture devices 118 can include a monitoring device 120, a wearable device 122, a lab-on-a-chip device 124, or one or more combinations thereof. One or more data capture devices 118 can be in communication with the computing device 112. For example, one or more of the data capture devices 118 can be in wireless communication with the computing device 112. In one or more illustrative examples, one or more of the data capture devices 118 can be in communication with the computing device 112 via a Bluetooth network or via a wireless local area network. Additionally, one or more of the data capture devices 118 can be in communication with the computing device 112 via a wired connection between the one or more data capture devices 118 and the computing device 112. To illustrate, one or more of the data capture devices 118 can be coupled to the computing device 112 using a Universal Serial Bus (USB) interface and/or a micro-USB interface. Although the one or more data capture devices 118 are shown in the illustrative example of FIG. 1 to be separate from the computing device 112, in various implementations, one or more components of the data capture devices 118 can be included in the computing device 112.

The monitoring device 120 can include a first component that couples to a portion of a body of the individual 114, such as a finger, a toe, or a portion of the ear. The first component of the monitoring device 120 can capture data that is sent to a second component of the monitoring device 120 that analyzes and displays the data captured by the first component. The monitoring device 120 can obtain health information associated with the individual 114. The health information can include blood-related information of the individual 114 that is obtained by using emitters of the monitoring device 120 that can emit electromagnetic radiation at one or more wavelengths and detectors of the monitoring device 120 that can detect at least a portion of the electromagnetic radiation emitted by the emitters.

The wearable device 122 can be worn by the individual 114 and can obtain health information about the individual 114. The wearable device 122 can be wrapped around a portion of the body of the individual 114. For example, the wearable device 122 can be wrapped around a finger of the individual 114. The wearable device 122 can also be worn on a finger or a toe of the individual 114. To illustrate, the wearable device 122 can be placed on and cover a finger of the individual 114. The wearable device 122 can obtain blood-related information of the individual 114 based on amounts of attenuation of electromagnetic radiation emitted by the wearable device 122.

The lab-on-a-chip device 124 can obtain health related information of the individual 114. In various implementations, a blood sample or a tissue sample can be placed in the lab-on-a-chip device 124. The lab-on-a-chip device 124 can include one or more sensors that can capture data about the individual 114 based on the blood sample or the tissue sample. In illustrative examples, the lab-on-a-chip device 124 can produce data that corresponds to blood-related information about the individual 114.

Blood-related information obtained by the one or more data capture devices 118 can indicate at least one of an amount of oxygenated hemoglobin in the blood of the individual 114 or an amount of de-oxygenated hemoglobin in the blood of the individual 114. The blood-related information can also indicate a packed cell volume of the individual 114. In addition, one or more of the one or more data capture devices 118 can obtain additional health information of the individual 114. For example, one or more of the data capture devices 118 can obtain information indicating blood oxygenation levels of the individual 114 and/or heart rate information of the individual 114. In various implementations, the one or more data capture devices 118 can be in communication with the health data system 102 via the one or more networks 116. For example, the one or more data capture devices 118 can send health-related information obtained by the one or more data capture devices 118 to the health data system 102 using the one or more networks 116.

The environment 100 can also include an additional computing device 126 that is operated by a healthcare practitioner 128. The additional computing device 126 can include a mobile computing device, a smart phone, a tablet computing device, a laptop computing device, a desktop computing device, one or more combinations thereof, and the like. The individual 114 can be a patient of the healthcare practitioner 128 and the healthcare practitioner 128 can provide treatment to the individual 114 for one or more biological conditions of the individual 114. In one or more implementations, the healthcare practitioner 128 can provide treatment to the individual 114 for a blood-related biological condition, such as anemia. The information obtained by the one or more data capture devices 118 corresponding to the individual 114 can be accessed by the healthcare practitioner 128 via the health data system 102 using the additional computing device 126.

In various implementations, the data extraction and processing system 106 can obtain information from one or more first data sources 130 and one or more second data sources 132. The one or more first data sources 130 and the one or more second data sources 132 can include one or more data stores that are electronically accessible by the health data system 102. For example, the health data system 102 can access the one or more first data sources 130 and the one or more second data sources 132 using at least one of one or more wireless communication networks or one or more wired communication networks. In one or more implementations, at least one of the one or more first data sources 130 or the one or more second data sources 132 can be remotely located with respect to the health data system 102. Additionally, at least one of the one or more first data sources 130 or the one or more second data sources 132 can be maintained by a same entity that implements the health data system 102. Further, at least one of the one or more first data sources 130 or the one or more second data sources 132 can be maintained by a different entity than the entity that maintains the health data system 102. The one or more first data sources 130 and/or the one or more second data sources 132 can include one or more publicly accessible databases. The one or more first data sources 130 and/or the one or more second data sources 132 can also include one or more privately maintained databases. In various implementations, the data stored by at least one of the one or more first data sources 130 or the one or more second data sources 132 can be accessed using one or more websites.

The one or more first data sources 130 can store health data 134 of a number of individuals, such as the individual 114. The health data 134 can be included in medical records of individuals. The health data 134 can also include data captured by the one or more data capture devices 118. The health data 134 can include blood analysis data 136. The blood analysis data 136 can include information that indicates characteristics of blood of individuals. For example, the blood analysis data 136 can include at least one of red blood cell count, packed cell volume, amount of oxygenated hemoglobin, amount of de-oxygenated hemoglobin, or blood oxygen levels. The health data 134 can also include additional health data 138 that is different from the blood analysis data 136. The additional health data 138 can include cardiovascular health information, such as heart rate information and blood pressure information. In addition, the additional health data 138 can include imaging information, laboratory test results, diagnostic test information, clinical observations, dental health information, one or more combinations thereof, and so forth.

Further, the one or more second data sources 132 can store medical literature information 140. The medical literature information 140 can include medical research information. For example, the medical literature information 140 can include clinical research information from a number of sources. To illustrate, the medical literature information 140 can include clinical research information obtained from clinical trials, academic medical research, healthcare provider medical research, enterprise medical research, one or more combinations thereof, and the like. In various implementations, the medical literature information 140 can include research papers, journal articles, video presentations, audio presentations, slides from presentations, websites, books, or one or more combinations thereof, that include health-related information. In one or more implementations, the medical literature information 140 can include information that is related to the diagnosis and treatment of anemia.

The data extraction and processing system 106 can obtain information from a number of data sources. For example, the data extraction and processing system 106 can obtain information from at least one of the one or more first data sources 130, the one or more second data sources 132, the computing device 112, the one or more data capture devices 118, or the additional computing device 126. In various examples, the data extraction and processing system 106 can obtain information that is sent to the health data system 102 from one or more data sources. In addition, the data extraction and processing system 106 can obtain information from one or more data sources using one or more queries. The one or more queries can be related to various types of information that can be stored by the one or more data sources. Further, the one or more queries can be associated with one or more keywords. In various implementations, the data extraction and processing system 106 can obtain information from one or more data sources using one or more application programming interface (API) calls. In situations where the data extraction and processing system 106 obtains information from at least one website, the data extraction and processing system 106 can use a web crawler to extract the information from the at least one website.

The data extraction and processing system 106 can process the data obtained from one or more data sources by encoding the data according to one or more formats. For example, the data extraction and processing system 106 can format data obtained from one or more data sources such that the data can be stored and retrieved from a database accessible to the health data system 102. In one or more illustrative examples, the data extraction and processing system 106 can format data obtained from one or more data sources according to one or more data structures. The data extraction and processing system 106 can also process data obtained from one or more data sources such that the data can be analyzed using the one or more machine learning systems 108 and/or included in communications generated by the data access and communication system 110.

In various implementations, the one or more machine learning systems 108 can analyze blood-related data to determine a level of anemia for individuals or a probability of anemia being present in the individuals. For example, the one or more machine learning systems 108 can analyze blood-related data of individuals, such as packed cell volume, amount of oxygenated hemoglobin, amount of de-oxygenated hemoglobin, with respect to one or more thresholds to determine a level of anemia for individuals or a probability of anemia being present in the individuals. The levels of anemia can indicate that an individual is anemic or that an individual is not anemic. The levels of anemia can also indicate that an individual has greater than a threshold probability of becoming anemic. In various examples, the information produced by the one or more machine learning systems 108 can include diagnostic health information that can be used by a healthcare practitioner to determine a diagnosis of an individual with respect to a biological condition. An indicator of the level of anemia for an individual and/or the probability of anemia being present in an individual can be used as diagnostic health information by a healthcare practitioner.

In one or more implementations, the one or more machine learning systems 108 can use genetic data, population data, location data, nutritional information, medical history information, or one or more combinations thereof, in conjunction with blood-related data to determine a level of anemia for respective individuals. The one or more machine learning systems 108 can also analyze blood-related data in conjunction with additional health data to determine a type of anemia for individuals. To illustrate, the one or more machine learning systems 108 can determine that a probability of an individual having sickle cell anemia is greater than a threshold probability based on blood-related information and genetic information. Further, the one or more machine learning systems 108 can analyze blood-related data and additional health information to determine a type of anemia for individuals and/or to determine one or more underlying conditions that can lead to anemia. In one or more illustrative examples, the one or more machine learning systems 108 can analyze blood-related data and additional health information, such as imaging data, clinical records, laboratory test results, and the like, to determine that an individual has anemia related to diabetes or anemia related to a bone marrow disease.

The one or more machine learning systems 108 can also determine recommendations for treatment of anemia. The one or more machine learning systems 108 can determine nutritional recommendations that can be used to treat anemia in one or more individuals. Additionally, the one or more machine learning systems 108 can identify one or more supplements and/or one or more medications that can be used to treat anemia in one or more individuals. In one or more implementations, the one or more machine learning systems 108 can determine a recommendation for one or more individuals to seek treatment from a healthcare practitioner for anemia and/or for an underlying cause of anemia. Further, the one or more machine learning systems 108 can determine an amount that anemia is present in one or more locations. For example, the one or more machine learning systems 108 can determine locations that have about an average number of incidents of anemia, locations that have less than an average number of incidents of anemia, and/or locations that have greater than an average number of incidents of anemia. The one or more machine learning systems 108 can also determine an amount that anemia is present in one or more populations. In various implementations, the one or more machine learning systems 108 can determine characteristics of individuals diagnosed with anemia that are different from blood-related characteristics of the individuals. To illustrate, the one or more machine learning systems 108 can determine genetic and/or morphological traits of individuals diagnosed with anemia. In addition, the one or more machine learning systems 108 can determine lifestyle characteristics of individuals diagnosed with anemia, such as nutritional practices or amounts of physical activity.

The one or more data access and communication systems 110 can provide access to information generated by and/or stored in association with the health data system 102. In one or more implementations, the one or more data access and communication systems 110 can cause blood-related data and other health-related information to be accessible to the computing device 112 and/or the additional computing device 126. For example, the one or more data access and communication systems 110 can provide at least portions of the blood analysis data 136 and/or at least portions of the additional health data 138 to at least one of the computing device 112 and/or the additional computing device 126. In this way, at least one of the individual 114 or the healthcare practitioner 128 can obtain information related to the diagnosis and/or treatment of anemia with respect to the individual 114. The one or more data access and communication systems 110 can also provide recommendations related to the treatment of anemia that have been determined by the one or more machine learning systems 108. Further, the one or more data access and communication systems 110 can provide communications to the computing device 112 and/or to the additional computing device 126 that includes reminders about treatments for anemia of the individual 114. For example, the one or more data access and communication systems 110 can provide reminders for the individual 114 to take a nutritional supplement to treat anemia. The one or more data access and communication systems 110 can also provide alerts to the computing device 112 and/or to the additional computing device 126 indicating that the individual 114 has not taken a nutritional supplement or followed some other treatment related to anemia. The one or more data access and communication systems 110 can send communications that include alerts and/or reminders that include email, text messages, video messages, audio messages, automated phone calls, one or more combinations thereof, and so forth.

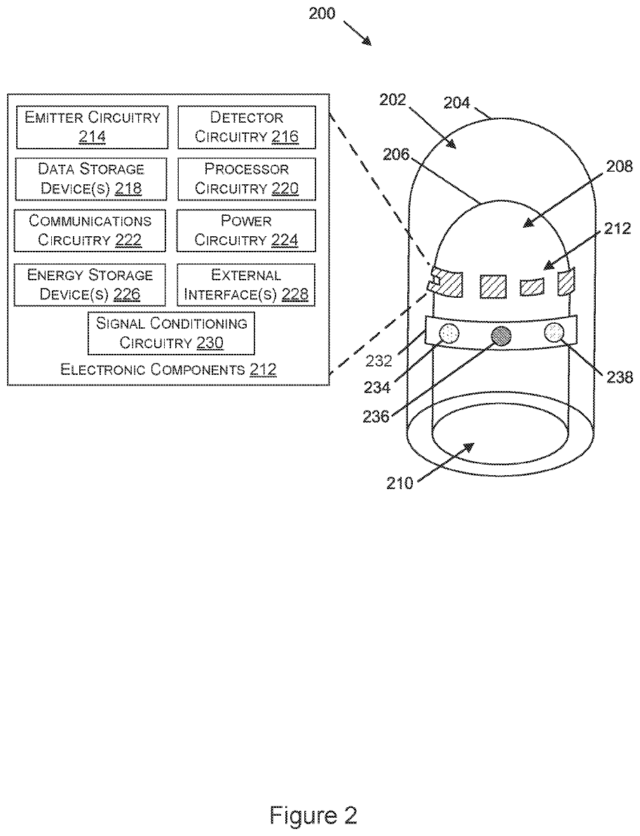

FIG. 2 illustrates an example wearable device 200 that includes circuitry to detect and analyze levels of hemoglobin metalloproteins and to determine hematocrit levels present in the blood of individuals. The wearable device 200 can include a casing 202 that includes an outer surface 204 and an inner surface 206. The casing 202 can define a lumen 208 that is accessible via an opening 210 in the casing 202. In various implementations, the lumen 208 and the opening 210 can be sized such that the wearable device 200 can be placed on a finger of an individual. In the illustrative example of FIG. 2, the wearable device 200 can correspond to the shape of a finger of an individual by having an elongated cylindrical region that terminates in a domed region. The lumen 208 can have a shape that is substantially similar to that of the outer surface 204 of the wearable device 200 and can include an elongated cylindrical region that terminates in a domed region. In one or more illustrative examples, the lumen 208 and the opening 210 can be sized such that the wearable device 200 can be placed on an index finger of an individual. The casing 202 can be comprised of a material. For example, the casing 202 can be comprised of a polymeric material. In one or more illustrative examples, the casing 202 can be comprised of a silicone-containing material. In additional examples, the casing 202 can be comprised of a fabric material. In further examples, the casing 202 can be made using an additive manufacturing process and the casing 202 can be comprised of a polymeric material that is suitable for use in one or more additive manufacturing processes.

The wearable device 200 can include electronic components 212. The electronic components 212 can be disposed within the casing 202. In one or more illustrative examples, the electronic components 212 can be disposed radially around at least a portion of the lumen 208. In various implementations, the electronic components 212 can be encased within the casing 202. Although not shown in the illustrative example of FIG. 2, at least a portion of the electronic components 212 can be electrically coupled using one or more electrical connectors. The electronic components 212 can include circuitry, logic, additional electronic components, or one or more combinations thereof, that can operate to capture and analyze blood-related information of individuals. In various implementations, the electronic components 212 can also include circuitry, logic, additional electronic components, or one or more combinations thereof, that can operate to capture and analyze additional health-related information.

The electronic components 212 can include emitter circuitry 214. The emitter circuitry 214 can cause electromagnetic radiation to be emitted. For example, the emitter circuitry 214 can include one or more light emitting diodes (LEDs). In various implementations, the emitter circuitry 214 can cause electromagnetic radiation of a number of wavelengths to be emitted. For example, the emitter circuitry 214 can include one or more emitters that emit wavelengths of electromagnetic radiation according to a plurality of different ranges of wavelengths with each particular range of wavelengths being a subset of wavelengths included in a broader set of wavelengths that spans from about 300 nm to about 1400 nm. To illustrate, the emitter circuitry 214 can emit electromagnetic radiation having wavelengths from about 370 nm to about 470 nm. In additional implementations, the emitter circuitry 214 can emit electromagnetic radiation having wavelengths from about 540 to about 690 nm. Further, the emitter circuitry 214 can emit electromagnetic radiation having wavelengths from about 710 nm to about 770 nm. In one or more implementations, the emitter circuitry 214 can emit electromagnetic radiation having wavelengths from about 780 nm to about 840 nm. In various examples, the emitter circuitry 214 can emit electromagnetic radiation having wavelengths from about 810 nm to about 900 nm. In one or more illustrative examples, the emitter circuitry 214 can emit electromagnetic radiation having wavelengths from about 910 nm to about 990 nm. The emitter circuitry 214 can also emit electromagnetic radiation having wavelengths from about 1200 nm to about 1400 nm.

The emitter circuitry 214 can include timing circuitry to determine timing of emission of electromagnetic radiation. The timing circuitry can determine a timing of emission of different wavelengths of electromagnetic radiation. To illustrate, the emitter circuitry 214 can include timing circuitry that causes different wavelengths of electromagnetic radiation to be emitted according to a pattern or timing sequence. In one or more implementations, the emitter circuitry 214 can include timing circuitry that causes various wavelengths of electromagnetic radiation to be emitted for a period of time according to the pattern or timing sequence. For example, the emitter circuitry 214 can include timing circuitry that causes a first range of wavelengths of electromagnetic radiation to be emitted during a first period of time followed by a second range of wavelengths of electromagnetic radiation to be emitted during a second period of time. In various implementations, the emitter circuitry 214 can include timing circuitry that causes a pause in electromagnetic radiation emission to be implemented between the first period of time and the second period of time.

The emitter circuitry 214 can be triggered to emit electromagnetic radiation based on receiving input. The input can include activation of one or more sensors included in the electronic components 212. In one or more illustrative examples, the emitter circuitry 214 can emit electromagnetic radiation in response to sensor input indicating that the wearable device 200 has been placed on a finger of an individual. Additionally, the emitter circuitry can stop emitting electromagnetic radiation in response to sensor input indicating that the wearable device 200 has been taken off of a finger of an individual. Further, the wearable device 200 can include an input device, such as a button or touch sensor, that can be activated to cause the emitter circuitry 214 to emit radiation and/or to stop emitting radiation. In various implementations, the emitter circuitry 214 can be controlled using a device that is remotely located from the wearable device 200 to emit electromagnetic radiation and/or to stop emitting electromagnetic radiation. To illustrate, a computing device that is in communication with the wearable device 200 can execute an application that can cause one or more signals to be sent to the wearable device 200 and that are received by the emitter circuitry 214. The one or more signals can cause the emitter circuitry 214 to emit electromagnetic radiation. The application can also send one or more additional signals to the wearable device 200 that are received by the emitter circuitry 214 and cause the emitter circuitry 214 to stop emitting electromagnetic radiation.

In one or more implementations, the emitter circuitry 214 can include timing circuitry that causes electromagnetic radiation to be emitted for a period of time of at least about 50 microseconds, at least about 100 microseconds, at least about 150 microseconds, at least about 200 microseconds, or at least about 250 microseconds. In addition, the emitter circuitry 214 can include timing circuitry that causes electromagnetic radiation to be emitted for no greater than about 500 microseconds, no greater than about 450 microseconds, no greater than about 400 microseconds, no greater than about 350 microseconds, or no greater than about 300 microseconds. In one or more illustrative examples, emitter circuitry 214 can include timing circuitry that causes electromagnetic radiation to be emitted for a period of time from about 50 microseconds to about 500 microseconds, from about 100 microseconds to about 400 microseconds, from about 50 microseconds to about 300 microseconds, or from about 50 microseconds to about 200 microseconds.

The electronic components 212 can also include detector circuitry 216 that detects electromagnetic radiation having a range of wavelengths. In one or more implementations, the detector circuitry 216 can detect electromagnetic radiation having wavelengths from about 300 nm to about 1400 nm. The detector circuitry 216 can include one or more photodiodes that detect electromagnetic radiation. In various implementations, the detector circuitry 216 can include a plurality of photodiodes. For example, the detector circuitry 216 can include a first photodiode that detects electromagnetic radiation having one or more first wavelengths emitted by a first LED of the emitter circuitry 214 and a second photodiode that detects electromagnetic radiation having one or more second wavelengths emitted by a second LED of the emitter circuitry 214. In one or more illustrative examples, the emitter circuitry 214 can include one or more first emitters that emit electromagnetic radiation having wavelengths from about 540 nm to about 690 nm, one or more second emitters that emit electromagnetic radiation having wavelengths from about 710 nm to about 770 nm, one or more third emitters that emit electromagnetic radiation having wavelengths from about 780 nm to about 840 nm, one or more fourth emitters that emit electromagnetic radiation from about 810 nm to about 900 nm, and one or more fifth emitters that emit electromagnetic radiation having wavelengths from about 910 nm to about 990 nm.

In addition, the electronic components 212 can include one or more data storage devices 218 and processor circuitry 220. The one or more data storage devices 218 can store data generated by the wearable device 200. For example, the one or more data storage devices 218 can store data corresponding to the emission of electromagnetic radiation. To illustrate, the one or more data storage devices 218 can store data indicating the emission of electromagnetic radiation of one or more wavelengths for one or more periods of time by the emitter circuitry 214. The one or more data storage devices 218 can also store wavelengths of electromagnetic detected by the detector circuitry 216 during one or more periods of time. In various implementations, the one or more data storage devices 218 can store data indicating an intensity of electromagnetic radiation emitted by the emitter circuitry 214 and an intensity of electromagnetic radiation detected by the detector circuitry 216. The one or more data storage devices 218 can include random access memory. In one or more implementations, the one or more data storage devices 218 can include read only memory. In additional implementations, the one or more data storage devices 218 can include flash memory.

The processor circuitry 220 can include one or more processors to execute computer-readable instructions. The computer-readable instructions can be stored by the one or more data storage devices 218. In one or more implementations, computer-readable instructions executed by the processor circuitry 220 can be stored by cache memory. In various implementations, the processor circuitry 220 can include a floating-point unit. In one or more illustrative examples, the processor circuitry 220 can execute computer-readable instructions that can analyze electromagnetic radiation wavelength emission data, electromagnetic wavelength detection data, electromagnetic wavelength attenuation data, or one or more combinations thereof, to determine a level of anemia or a probability of anemia being present in an individual wearing the wearable device 200. For example, the processor circuitry 220 can determine an amount of attenuation of electromagnetic radiation for one or more wavelengths and determine blood-related information based on the amount of attenuation. The amount of attenuation of the electromagnetic radiation for the one or more wavelengths can corresponds to the presence or absence of hemoglobin metalloproteins. The amount of hemoglobin metalloproteins present in the blood of an individual can be used by the processor circuitry 220 to determine a level of anemia or a probability of anemia being present in the individual.

Further, the electronic components 212 can include communications circuitry 222. The communications circuitry 222 can include transmission circuitry to transmit data and receiver circuitry to receive data. The communications circuitry 222 can also include one or more antennas. The communications circuitry 222 can transmit and receive data according to one or more communications protocols. For example, the communications circuitry 222 can transmit and receive data according to a Bluetooth communications protocol. The communications circuitry 222 can also transmit and receive data according to a wireless local area network communications protocol, such as one or more Institute of Electrical and Electronics Engineers (IEEE) 1102.11 protocols. In various implementations, the communications circuitry 222 can transmit and receive data according to one or more wide area wireless communication standards, such as one or more cellular communication standards.

The electronic components 212 can include power circuitry 224 and one or more energy storage devices 226. The power circuitry 224 can control the supply of power to one or more of the electronic components 212 from the one or more energy storage devices 226. In various implementations, the power circuitry 224 can control charging of the one or more energy storage devices 226. The one or more energy storage devices 226 can include at least one battery. In one or more implementations, the one or more energy storage devices 226 can include at least one supercapacitor. In one or more illustrative examples, the one or more energy storage devices 226 can include a battery supplying from about 1 volt (V) to about 5 volts. Additionally, the one or more energy storage devices 226 can include a rechargeable battery, such as a lithium-ion battery.

The electronic components 212 can also include one or more external interfaces 228. The one or more external interfaces 228 can provide a connection between the wearable device 200 and one or more external devices. For example, the one or more external interfaces 228 can provide a connection between the wearable device 200 and a monitoring device that collects data from the wearable device 200. Additionally, the one or more external interfaces 228 can provide a connection between the wearable device 200 and a mobile computing device, such as a smartphone and/or a tablet computing device. In one or more illustrative examples, the one or more external interfaces 228 can include a universal serial bus (USB) interface. In one or more additional illustrative examples, the one or more external interfaces 228 can include a micro-USB interface. In further examples, the one or more external interfaces 228 can include a Lightning interface or a Thunderbolt interface.

Additionally, the electronic components 212 can include signal conditioning circuitry 230. The signal conditioning circuitry 230 can include at least one of one or more filters or one or more amplifiers to modify signals received by the detector circuitry 216. The signal conditioning circuitry 230 can filter data related to the detection of electromagnetic radiation that is not emitted by the emitter circuitry 214. For example, the signal conditioning circuitry 230 can filter data obtained from the detector circuitry 216 that corresponds to ambient light in an environment that includes the wearable device 200.

In one or more implementations, the wearable device 200 can include an indicator band 232 that is disposed radially within the casing 202. The indicator band 232 can include one or more indicators that can individually correspond to a level of anemia. For example, the indicator band 232 can include a first indicator 234 that corresponds to a first level of anemia, a second indicator 236 that corresponds to a second level of anemia, and a third indicator 238 that corresponds to a third level of anemia. Each level of anemia can correspond to a range of probabilities that an individual is anemic. In one or more illustrative examples, a first level of anemia can correspond to relatively low probabilities that an individual is anemic, a second level of anemia can correspond to intermediate probabilities that an individual is anemic, and a third level of anemia can correspond to relatively high probabilities that an individual is anemic. In one or more additional implementations, each level of anemia can correspond to a level of need for the individual to receive treatment for anemia. To illustrate, a first level of anemia can correspond to a relatively low level of need to receive treatment for anemia, a second level of anemia can correspond to an intermediate level of need to receive treatment for anemia, and a third level of anemia can correspond to a relatively high level of need to receive treatment for anemia.

The indicator band 232 can be comprised of a polymeric material. Additionally, the indicator band 232 can be comprised of a metallic material. Further, the indicators 234, 236, 238 can comprise LEDs. In various implementations, each indicator 234, 236, 238 can comprise LEDs that emit wavelengths of electromagnetic radiation that correspond to different colors. To illustrate, the first indicator 234 can include an LED that emits wavelengths of electromagnetic radiation that correspond to a green color, the second indicator 236 can include an LED that emits wavelengths of electromagnetic radiation that correspond to a yellow color, and the third indicator 238 can include an LED that emits wavelengths of electromagnetic radiation that correspond to a red color. Although the indicator band 232 of the illustrative example of FIG. 2 includes three indicators, in additional examples, the indicator band 232 can include more indicator bands or fewer indicator bands. To illustrate, the indicator band 232 can include a first indicator and a second indicator.

The indicators 234, 236, 238 can be coupled to one or more of the electronic components 212. In various implementations, the indicators 234, 236, 238 can be activated by the processor circuitry 220. For example, after the wearable device 200 is placed on a finger of an individual, the wearable device 200 can collect data from the emitter circuitry 214 and the detector circuitry 216. The processor circuitry 220 can execute computer-readable instructions to analyze the data and determine a level of anemia of the individual. The processor circuitry 220 can determine which one of the indicators 234, 236, 238 corresponds to the level of anemia and then activate the respective indicator 234, 236, or 238.

Although in the illustrative example of FIG. 2 the wearable device 200 has a shape that corresponds to that of a finger of an individual, in additional implementations, the wearable device 200 can have other shapes. For example, the wearable device 200 can be formed in the shape of a band that can wrap around a body part of an individual, such as a finger or a toe of the individual. In these implementations, the wearable device 200 can comprise an elongated cylindrical region without the domed region such that the wearable device 200 is open at the top and bottom when wrapped around a body part of an individual.

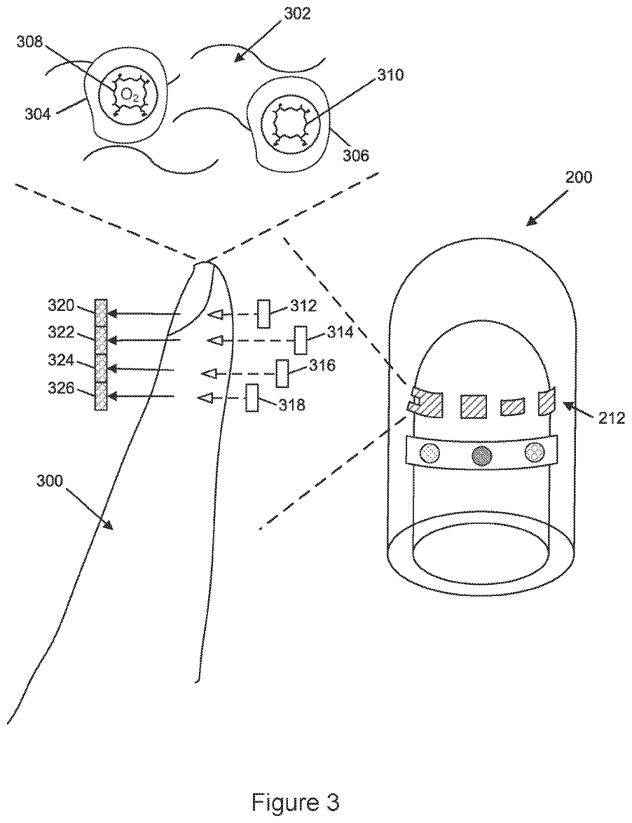

FIG. 3 illustrates an example wearable device 200 that can be placed on a finger 300 and that includes an array of emitters that each transmit electromagnetic radiation at different wavelengths and one or more detectors that detect electromagnetic radiation produced by the array of emitters. The illustrative example of FIG. 3 shows a portion of blood 302 that can be flowing through the finger 300. The blood 302 can include a first red blood cell 304 and a second red blood cell 306. The red blood cells 304, 306 can include a number of biomolecules. For example, the first red blood cell 304 can include a number of hemoglobin molecules, such as the first hemoglobin molecule 308, and the second red blood cell 306 can include an additional number of hemoglobin molecules, such as the second hemoglobin molecule 310. The first hemoglobin molecule 308 is bound to an oxygen molecule and the second hemoglobin molecule 310 is not bound to an oxygen molecule. Hemoglobin molecules that are bound to at least one oxygen molecule can be referred to herein as oxygenated hemoglobin. In addition, hemoglobin molecules that are not bound to an oxygen molecule can be referred to herein as de-oxygenated hemoglobin.

The wearable device 200 can include electronic components 212. The electronic components 212 can include emitter circuitry that includes a number of emitters 312, 314, 316, 318 and detector circuitry that includes one or more detectors 320, 322, 324, 326. The number of emitters 312, 314, 316, 318 can include LEDs that emit different wavelengths of electromagnetic radiation and the one or more detectors 320, 322, 324, 326 can include photodiodes that can detect the wavelengths of electromagnetic radiation emitted by the emitters. The emitters 312, 314, 316, 318 can also emit electromagnetic radiation with one or more intensities. In addition, the detectors 320, 322, 324, 326 can detect one or more intensities of electromagnetic radiation. In the illustrative example of FIG. 3, the electronic components 212 can include a first emitter 312 that can be paired with a first detector 320, a second emitter 314 that can be paired with a second detector 322, a third emitter 316 that can be paired with a third detector 324, and a fourth emitter 318 that can be paired with a fourth detector 326. Although the illustrative example of FIG. 3 indicates four different detectors 320, 322, 324, 326, in additional implementations, the detector circuitry can include a different number of detectors. In an additional example, the detector circuitry can include a single detector that detects electromagnetic radiation emitted by the emitters 312, 314, 316, 318.

The first emitter 312 can emit a first range of wavelengths of electromagnetic radiation, the second emitter 314 can emit a second range of wavelengths of electromagnetic radiation, the third emitter 316 can emit a third range of wavelengths of electromagnetic radiation, and the fourth emitter 318 can emit a fourth range of wavelengths of electromagnetic radiation. In one or more illustrative examples, the first emitter 312 can emit wavelengths of electromagnetic radiation from about 540 nm to about 690 nm. In addition, the second emitter 314 can emit wavelengths of electromagnetic radiation from about 710 to about 770 nm. Further, the third emitter 316 can emit wavelengths of electromagnetic radiation from about 780 nm to about 840 nm. The fourth emitter 318 can emit wavelengths of electromagnetic radiation from about 910 nm to about 990 nm.

The presence or absence of various molecules within the blood 302 can impact the attenuation of electromagnetic radiation transmitted from the emitters 312, 314, 316, 318. For example, oxygenated hemoglobin can absorb electromagnetic radiation at a first set of wavelengths and de-oxygenated hemoglobin can absorb electromagnetic radiation at a second set of wavelengths that is different from the first set of wavelengths. By determining an amount of attenuation of various wavelengths of electromagnetic radiation emitted by the emitters 312, 314, 316, 318 based on electromagnetic radiation detected by one or more of the detectors 320, 322, 324, 326, the wearable device 200 can determine blood-related information for an individual.

In one or more illustrative examples, a protocol for the emission and detection of electromagnetic radiation by the wearable device 200 can be followed to determine information corresponding to the blood 302. For example, the first emitter 312 can emit first electromagnetic radiation having a first range of wavelengths for a first period of time and one or more of the detectors 320, 322, 324, 326 can detect an amount of the first electromagnetic radiation that is not absorbed by one or more types of molecules included in the blood 302. After the first emitter 312 has completed emitting electromagnetic radiation, a pause can occur for a period of time and the second emitter 314 can emit second electromagnetic radiation having a second range of wavelengths for a second period of time after the pause. One or more of the detectors 320, 322, 324, 326 can detect an amount of the second electromagnetic radiation that is not absorbed by one or more types of molecules included in the blood 302 Δn additional pause can follow the emission of the second electromagnetic radiation and after the additional pause, the third emitter 316 can emit third electromagnetic radiation having a third range of wavelengths for a third period of time. One or more of the detectors 320, 322, 324, 326 can detect an amount of the third electromagnetic radiation that is not absorbed by one or more types of molecules in the blood 302. Further, following a pause after the third period of time, the fourth emitter 318 can emit fourth electromagnetic radiation having a fourth range of wavelengths for a fourth period of time. In various implementations, one or more of the detectors 320, 322, 324, 326 can detect an amount of the fourth electromagnetic radiation that is not absorbed by one or more types of molecules included in the blood 302.

After implementing a protocol regarding the emission of electromagnetic radiation by the emitters 312, 314, 316, 318 and detection of electromagnetic radiation by one or more of the detectors 320, 322, 324, 326, the amount of attenuation of each of the ranges of electromagnetic radiation can be determined by the wearable device 200. The amount of attenuation of each of the ranges of electromagnetic radiation can indicate the presence or absence of one or more biomolecules in the blood 302. By analyzing the respective amounts of the one or more biomolecules in the blood 302, a level of anemia can be determined for an individual. For example, an amount of oxygenated hemoglobin, an amount of de-oxygenated hemoglobin, and a packed cell volume of the blood 302 can be determined based on the attenuation of electromagnetic radiation emitted by the emitters 312, 314, 316, 318. The amount of oxygenated hemoglobin, an amount of de-oxygenated hemoglobin, and a packed cell volume of the blood 302 can then be used to determine a level of anemia of an individual in which the blood 302 is flowing.

In one or more illustrative examples, the attenuation of electromagnetic radiation determined based on data produced by one or more of the emitters 312, 314, 316, 318 and data produced by one or more of the detectors 320, 322, 324, 326 can be used to determine a level of anemia or a probability of anemia being present in an individual. For example, an amount of attenuation of electromagnetic radiation at wavelengths from about 850 nm to about 1000 nm, such as from about 910 nm to about 990 nm, can indicate an amount of oxygenated hemoglobin present in the blood of individuals and an amount of attenuation of electromagnetic radiation at wavelengths from about 600 nm to about 750 nm, such as from about 600 nm to about 680 nm or from about 710 nm to about 790 nm, can indicate an amount of deoxygenated hemoglobin present in the blood of individuals. One or more correlations can be made between the amount of oxygenated hemoglobin and/or the amount of deoxygenated hemoglobin present in the blood of individuals with a level of anemia or having at least a threshold probability of being diagnosed with anemia. In various examples, the correlations can be made using previously obtained oxygenated hemoglobin levels and/or deoxygenated hemoglobin levels of individuals for which a respective level of anemia has been determined. To illustrate, a first level of oxygenated hemoglobin and/or a first level of deoxygenated hemoglobin can be correlated to a first level of anemia or a first range of probabilities of anemia being present in individuals based on information obtained from individuals previously diagnosed with anemia and a second level of oxygenated hemoglobin and/or a second level of deoxygenated hemoglobin can be correlated to a second level of anemia or a second range of probabilities of anemia being present in individuals based on information obtained from individuals previously diagnosed with anemia. The amounts of deoxygenated hemoglobin and/or oxygenated hemoglobin determined based on data obtained from one or more of the emitters 312, 314, 316, 318 and one or more of the detectors 320, 322, 324, 326 can then be compared against the predetermined correlations to determine a level of anemia or a probability of anemia being present in an individual wearing the wearable device 200.

In one or more implementations, the correlations between amounts of deoxygenated hemoglobin and oxygenated hemoglobin that correspond to levels of anemia or probabilities of being diagnosed with anemia can be expressed as one or more ratios. In various examples, the amount of attenuation of electromagnetic radiation of one or more wavelengths can be expressed as an amount of current produced by the one or more detectors 320, 322, 324, 326 based on an amount of electromagnetic radiation incident on the one or more detectors 320, 322, 324, 326 or an amount of voltage derived from the current produced by the one or more detectors 320, 322, 324, 326. In one or more illustrative examples, the correlations used to determine a level of anemia can be based on at least one of a direct current (DC) component or an alternating current (AC) component of the current derived from the one or more detectors 320, 322, 324, 326 based on electromagnetic radiation sensed by the one or more detectors 320, 322, 324, 326. Current and/or voltage readings can also be determined in response to the emission of electromagnetic radiation of the emitters 312, 314, 316, 318. In these situations, ratios of current and/or ratios of voltage related to the emission and the detection of electromagnetic radiation by the wearable device can be used to determine a level of anemia or a probability of anemia being present based on previously determined correlations between respective levels of anemia or respective probabilities of anemia being present and one or more current ratios and/or voltage ratios. In additional implementations, intensity of electromagnetic radiation emitted by one or more of the emitters 312, 314, 316, 318 and intensity of electromagnetic radiation detected by one or more of the detectors 320, 322, 324, 326 can also be correlated to levels of anemia. For example, ratios of intensity of emitted electromagnetic radiation and intensity of detected electromagnetic radiation can be used to determine a level of anemia or a probability of anemia being present based on previously determined correlations between levels of anemia or probabilities of anemia being present and ratios of intensity of emitted electromagnetic radiation with respect to intensity of detected electromagnetic radiation.

Although the illustrative example of FIG. 3 indicates that the wearable device 200 includes four emitters 312, 314, 316, 318 and a number of detectors 320, 322, 324, 326 to detect the electromagnetic radiation emitted, by the emitters 312, 314, 316, 318, the wearable device 200 can include a greater number of emitters and/or detectors or fewer emitters and/or detectors. In one or more examples, the wearable device 200 can include a fifth emitter that emits electromagnetic radiation having wavelengths from about 810 nm to about 900 nm and a fifth detector that detects electromagnetic radiation emitted by the fifth emitter. In one or more additional examples, the detectors 320, 322, 324, 326 and any other detectors included in the wearable device 200 can be included in a single photodiode that detects a range of electromagnetic radiation emitted by the emitters included in the wearable device 200, such as from about 500 nm to about 1400 nm or from about 600 nm to about 1000 nm.

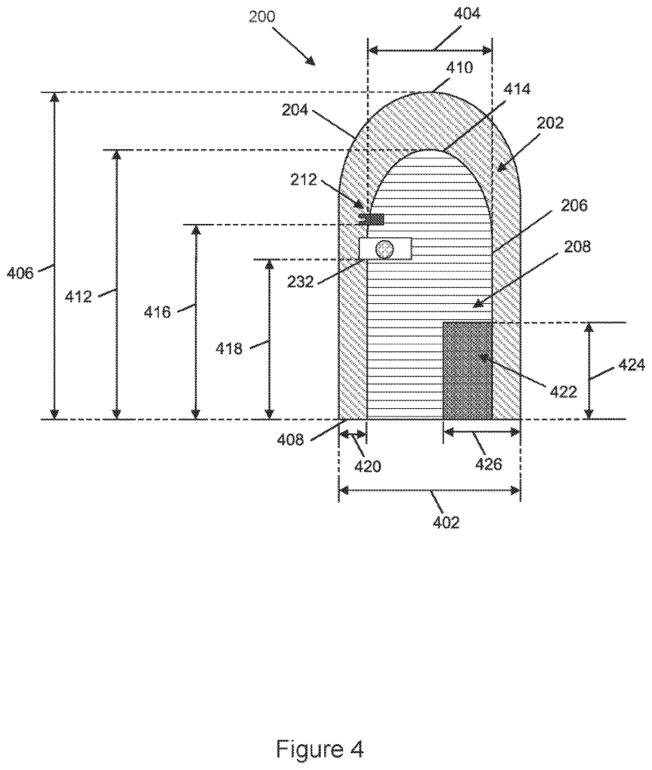

FIG. 4 illustrates a side view of an example wearable device 200 that generates data that can be used to determine a level of anemia of an individual. The casing 202 of the wearable device 200 can have a width 402 that is from about 0.2 centimeters (cm) to about 10 cm, from about 0.7 cm to about 2.5 cm, from about 1.8 cm to about 4 cm, from about 2.5 cm to about 5 cm, from about 3 cm to about 6.5 cm, from about 5 cm to about 9 cm, or from about 1 cm to about 5 cm. In addition, the lumen 208 can have a width 404 that is from about 0.05 cm to about 6 cm, from about 0.5 cm to about 1.5 cm, from about 1.5 cm to about 3 cm, from about 2 cm to about 4.5 cm, from about 0.1 cm to about 4 cm, from about 3 cm to about 5 cm, or from about 0.8 cm to about 4.5 cm. The width 404 of the lumen 208 can correspond to a width of the opening 210 of the wearable device 200.

The wearable device 200 can also have a height 406 from a base 408 of the wearable device 200 to an apex 410 of the wearable device 200. The height 406 can be from about 2 cm to about 10 cm, from about 2 cm to about 3.6 cm, from about 2.8 cm to about 5 cm, from about 4 cm to about 6.4 cm, or from about 5 cm to about 10 cm. Additionally, the lumen 208 can have a height 412 from the base 408 of the wearable device 200 to an apex 414 of the lumen 208 from about 1.5 cm to about 8 cm, from about 1.5 cm to about 3 cm, from about 2.5 cm to about 4.5 cm, from about 3.6 cm to about 5.5, and from about 4.5 cm to about 8 cm. Further, the wearable device 200 can include a height 416 from a bottom of one of the electronic components 212 to the base 408 of the wearable device 200. The height 416 can be from about 0.8 cm to about 5.5 cm, from about 0.8 cm to about 2 cm, from about 1.4 cm to about 2.8 cm, from about 2.5 cm to about 4 cm, from about 3.5 cm to about 5 cm, or from about 4 cm to about 5.5 cm. The wearable device 200 can also have a height 418 from a bottom of the indicator band 232 to the base 408 of the wearable device 200 that is from about 0.7 cm to about 5 cm, from about 0.7 cm to about 2 cm, from about 1.4 cm to about 2.7 cm, from about 2.3 cm to about 4 cm, or from about 2.8 cm to about 5 cm. The casing 202 can have a width 420 from the outer surface 204 of the wearable device 200 to the inner surface 206 of the lumen 208 that is from about 0.10 cm to about 3 cm, from about 0.1 cm to about 1 cm, from about 0.6 cm to about 1.5 cm, or from about 1.2 cm to about 2.5 cm, or from about 1.8 cm to about 3 cm.

In various implementations, the wearable device 200 can include a cut-out region 422. The cut-out region 422 can be free of the material comprising the wearable device 200. The cut-out region 422 can have a height 424 and a width 426. The height 424 of the cut-out region 422 can be from about 0.8 cm to about 4 cm, from about 0.8 cm to about 2.3 cm, from about 1.4 cm to about 2.8, or from about 2.5 cm to about 4 cm. The width 426 of the cut-out region 422 can be from about 0.5 cm to about 3 cm, from about 0.5 cm to about 2 cm, or from about 1.5 cm to about 3 cm.

In one or more illustrative examples, the wearable device 200 can have a height 406 from about 5 cm to about 9 cm, the lumen 208 can have a height 412 from about 3.5 cm to about 5.5 cm, the height 416 of the wearable device from a bottom of one of the electronic components 212 to the base 408 of the wearable device 200 can be from about 3.3 cm to about 5.3 cm, the height 418 from a bottom of the indicator band 232 to the base 408 of the wearable device 200 can be from about 2 cm to about 4 cm, the width 420 of the casing 202 from the outer surface 204 of the wearable device 200 to the inner surface 206 of the lumen 208 can be from about 1.2 cm to about 2.5 cm, the height 424 of the cut-out region 422 can be from about 1.2 cm to about 2.8 cm, and the width 426 of the cut-out region 422 can be from about 1 cm to about 2.2 cm.

In various implementations, the wearable device 200 can have dimensions that correspond to different sizes of fingers of individuals. For example, one or more first implementations of the wearable device 200 can have dimensions that correspond to those of a finger of a child, while one or more second implementations of the wearable device 200 can have dimensions that correspond to those of a finger of an adult. In one or more additional implementations, the wearable device 200 can have dimensions that correspond to those of a toe of a child or to a toe of an adult.

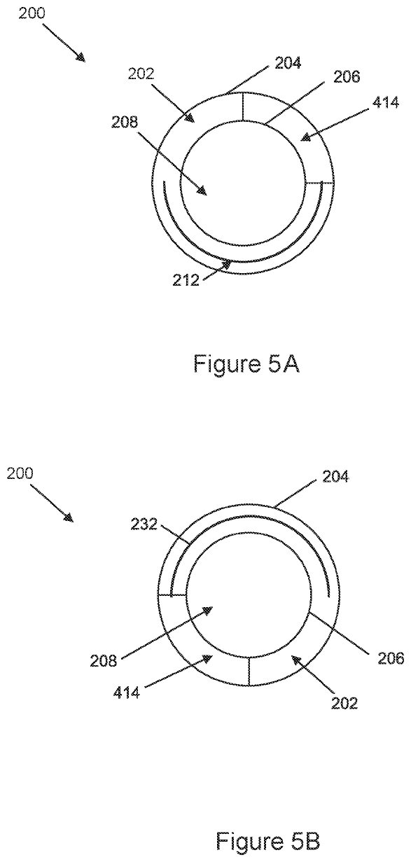

FIG. 5A illustrates a top view of an example wearable device 200 that generates data that can be used to determine a level of anemia or a probability of anemia being present in an individual. The top view of the wearable device 200 shows the casing 202 and the lumen 208 that is defined by the outer surface 204 and the inner surface 206. The top view of the wearable device 200 also shows the electronic components 212 and the cut-out region 422.

FIG. 5B illustrates a bottom view of an example wearable device 200 that generates data that can be used to determine a level of anemia or a probability of anemia being present in an individual. The bottom view of the wearable device 200 shows the casing 202 and the lumen 208 that is defined by the outer surface 204 and the inner surface 206. Additionally, the bottom view of the wearable device 200 also shows the indicator band 232 and the cut.

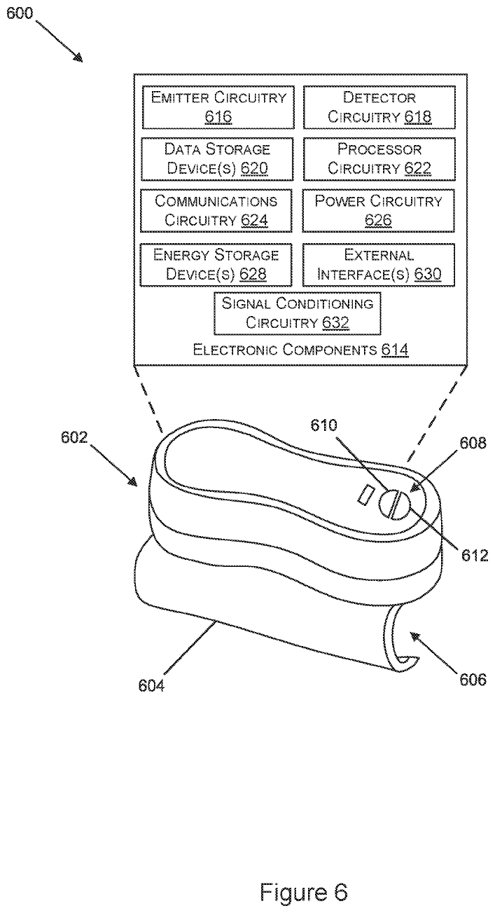

FIG. 6 illustrates another example wearable device 600 that includes sensor circuitry to detect levels of hemoglobin metalloproteins and to determine hematocrit levels present in the blood of individuals with a clamp member to attach to a finger of an individual. The wearable device 600 can include a body 602 and a clamp member 604. The clamp member 604 can have a concave shape and form an opening 606. The clamp member 604 can be shaped such that the wearable device 600 can clamp to a finger of an individual and minimize motion of the body 602 while the wearable device 600 is clamped to the finger of the individual. In one or more illustrative examples, the clamp member 604 and the opening 606 can be sized such that the wearable device 600 can be placed on an index finger of an individual.

In one or more examples, the body 602 and the clamp member 604 can form a uniform and continuous unit. In various examples, the clamp member 604 may not be formed such that the clamp member 604 is easily separated from the body 602. In one or more additional examples, the body 602 and the clamp member 604 can include an attachment mechanism such that the clamp member 604 can be separated from the body 602 without breaking at least one of the clamp member 604 or the body 602. In one or more illustrative examples, the attachment mechanism can include one or more grooves and one or more rails to attach and to detach the clamp member 604 to the body 602.

The body 602 and the clamp member 604 can be comprised of one or more materials. In various examples, the body 602 and the clamp member 604 can be comprised of a same material. In one or more additional examples, the body 602 and the clamp member 604 can be comprised of at least one different material. In one or more examples, at least one of the body 602 or the clamp member 604 can be comprised of a polymeric material. In one or more illustrative examples, at least one of the body 602 or the clamp member 604 can be comprised of a silicone-containing material. In additional examples, at least one of the body 602 or the clamp member 604 can be comprised of a fabric material. In further examples, at least one of the body 602 or the clamp member 604 can be made using an additive manufacturing process and at least one of the body 602 or the clamp member 604 can be comprised of a polymeric material that is suitable for use in one or more additive manufacturing processes.

The wearable device 600 can also include a number of user interface components 608. In the illustrative example of FIG. 6, the number of user interface components 608 can include at least a first user interface component 610 and a second user interface component 612. In one or more examples, at least a portion of the number or user interface components 608 can be selectable by an individual to cause the wearable device 600 to perform one or more functions. In one or more additional examples, at least a portion of the number of user interface components 608 can include one or more indicators, such as an indicator of a blood-oxygen level or an indicator of a probability of anemia being present in an individual wearing the wearable device 600. In various examples, at least a portion of the user interface components 608 can be displayed on a display device of the wearable device 600. In these scenarios, a portion of the body 602 can comprise a touch screen to display one or more of the user interface components 608 and to capture input related to one or more of the user interface components 608. In one or more further examples, at least a portion of the number of user interface components 608 can include physical components, such as buttons, that can be pressed by an individual to cause the wearable device 600 to perform one or more functions.

Additionally, at least a portion of the number of user interface components 608 can include indicators, such as one or more lighting components (e.g., light emitting diodes), that can provide an indicator of a biological condition of an individual based on a color shown by the one or more lighting components. For example, the number of user interface components 608 can include display one or more indicators that correspond to a first level of anemia, a second level of anemia, and a third level of anemia. Each level of anemia can correspond to a range of probabilities that an individual is anemic. In one or more illustrative examples, a first level of anemia can correspond to relatively low probabilities that an individual is anemic, a second level of anemia can correspond to intermediate probabilities that an individual is anemic, and a third level of anemia can correspond to relatively high probabilities that an individual is anemic. In one or more additional implementations, each level of anemia can correspond to a level of need for the individual to receive treatment for anemia. To illustrate, a first level of anemia can correspond to a relatively low level of need to receive treatment for anemia, a second level of anemia can correspond to an intermediate level of need to receive treatment for anemia, and a third level of anemia can correspond to a relatively high level of need to receive treatment for anemia.

In one or more examples, the number of user interface component 608 can comprise one or more LEDs that emit wavelengths of electromagnetic radiation that correspond to different colors and the different colors can correspond to a respective level of anemia. To illustrate, a first indicator of the number of user interface component 608 can include an LED that emits wavelengths of electromagnetic radiation that correspond to a green color and a second indicator of the number of user interface components 608 can include an LED that emits wavelengths of electromagnetic radiation that correspond to a red color. In one or more additional examples, the number of user interface components 608 can include a third indicator that comprises an LED that emits wavelengths of electromagnetic radiation that correspond to a yellow color.

The wearable device 600 can also include electronic components 614. The electronic components 614 can be disposed within the body 602. Although not shown in the illustrative example of FIG. 6, at least a portion of the electronic components 614 can be electrically coupled using one or more electrical connectors. The electronic components 614 can include circuitry, logic, additional electronic components, or one or more combinations thereof, that can operate to capture and analyze blood-related information of individuals. In various implementations, the electronic components 614 can also include circuitry, logic, additional electronic components, or one or more combinations thereof, that can operate to capture and analyze additional health-related information.

The electronic components 614 can include emitter circuitry 616. The emitter circuitry 616 can cause electromagnetic radiation to be emitted. For example, the emitter circuitry 616 can include one or more light emitting diodes (LEDs). In various implementations, the emitter circuitry 616 can cause electromagnetic radiation of a number of wavelengths to be emitted. For example, the emitter circuitry 616 can include one or more emitters that emit wavelengths of electromagnetic radiation according to a plurality of different ranges of wavelengths with each particular range of wavelengths being a subset of wavelengths included in a broader set of wavelengths that spans from about 300 nm to about 1400 nm. To illustrate, the emitter circuitry 616 can emit electromagnetic radiation having wavelengths from about 370 nm to about 470 nm. In additional implementations, the emitter circuitry 616 can emit electromagnetic radiation having wavelengths from about 600 to about 700 nm. Further, the emitter circuitry 616 can emit electromagnetic radiation having wavelengths from about 710 nm to about 790 nm. In one or more implementations, the emitter circuitry 616 can emit electromagnetic radiation having wavelengths from about 780 nm to about 840 nm. In one or more illustrative examples, the emitter circuitry 616 can emit electromagnetic radiation having wavelengths from about 810 nm to about 900 nm. In one or more additional illustrative examples, the emitter circuitry 616 can emit electromagnetic radiation having wavelengths from about 910 nm to about 1000 nm. The emitter circuitry 214 can also emit electromagnetic radiation having wavelengths from about 1200 nm to about 1400 nm. In one or more illustrative examples, the emitter circuitry 616 can include one or more first emitters that emit electromagnetic radiation having wavelengths from about 620 nm to about 700 nm, one or more second emitters that emit electromagnetic radiation having wavelengths from about 710 nm to about 790 nm, one or more third emitters that emit electromagnetic radiation having wavelengths from about 780 nm to about 840 nm, one or more fourth emitters that emit electromagnetic radiation from about 810 nm to about 890 nm, and one or more fifth emitters that emit electromagnetic radiation having wavelengths from about 910 nm to about 990 nm.

The emitter circuitry 616 can include timing circuitry to determine timing of emission of electromagnetic radiation. The timing circuitry can determine a timing of emission of different wavelengths of electromagnetic radiation. To illustrate, the emitter circuitry 616 can include timing circuitry that causes different wavelengths of electromagnetic radiation to be emitted according to a pattern or timing sequence. In one or more implementations, the emitter circuitry 616 can include timing circuitry that causes various wavelengths of electromagnetic radiation to be emitted for a period of time according to the pattern or timing sequence. For example, the emitter circuitry 616 can include timing circuitry that causes a first range of wavelengths of electromagnetic radiation to be emitted during a first period of time followed by a second range of wavelengths of electromagnetic radiation to be emitted during a second period of time. In various implementations, the emitter circuitry 616 can include timing circuitry that causes a pause in electromagnetic radiation emission to be implemented between the first period of time and the second period of time.

The emitter circuitry 616 can be triggered to emit electromagnetic radiation based on receiving input. The input can include activation of one or more sensors included in the electronic components 614. In one or more illustrative examples, the emitter circuitry 616 can emit electromagnetic radiation in response to sensor input indicating that the wearable device 600 has been placed on a finger of an individual. Additionally, the emitter circuitry 616 can stop emitting electromagnetic radiation in response to sensor input indicating that the wearable device 600 has been taken off of a finger of an individual. Further, the wearable device 600 can include a user interface component 608, such as a button or touch sensor, that can be activated to cause the emitter circuitry 616 to emit radiation and/or to stop emitting radiation. In various implementations, the emitter circuitry 616 can be controlled using a device that is remotely located from the wearable device 600 to emit electromagnetic radiation and/or to stop emitting electromagnetic radiation. To illustrate, a computing device that is in communication with the wearable device 600 can execute an application that can cause one or more signals to be sent to the wearable device 600 and that are received by the emitter circuitry 616. The one or more signals can cause the emitter circuitry 616 to emit electromagnetic radiation. The application can also send one or more additional signals to the wearable device 600 that are received by the emitter circuitry 616 and cause the emitter circuitry 616 to stop emitting electromagnetic radiation.

In one or more implementations, the emitter circuitry 616 can include timing circuitry that causes electromagnetic radiation to be emitted for a period of time of at least about 50 microseconds, at least about 100 microseconds, at least about 150 microseconds, at least about 200 microseconds, or at least about 250 microseconds. In addition, the emitter circuitry 616 can include timing circuitry that causes electromagnetic radiation to be emitted for no greater than about 500 microseconds, no greater than about 450 microseconds, no greater than about 400 microseconds, no greater than about 350 microseconds, or no greater than about 300 microseconds. In one or more illustrative examples, emitter circuitry 616 can include timing circuitry that causes electromagnetic radiation to be emitted for a period of time from about 50 microseconds to about 500 microseconds, from about 100 microseconds to about 400 microseconds, from about 50 microseconds to about 300 microseconds, or from about 50 microseconds to about 200 microseconds.

The electronic components 614 can also include detector circuitry 618 that detects electromagnetic radiation having a range of wavelengths. In one or more implementations, the detector circuitry 618 can detect electromagnetic radiation having wavelengths from about 300 nm to about 1400 nm. The detector circuitry 618 can include one or more photodiodes that detect electromagnetic radiation. In various implementations, the detector circuitry 618 can include a plurality of photodiodes. For example, the detector circuitry 618 can include a first photodiode that detects electromagnetic radiation having one or more first wavelengths emitted by a first LED of the emitter circuitry 616 and a second photodiode that detects electromagnetic radiation having one or more second wavelengths emitted by a second LED of the emitter circuitry 616.

In addition, the electronic components 614 can include one or more data storage devices 620 and processor circuitry 622. The one or more data storage devices 620 can store data generated by the wearable device 600. For example, the one or more data storage devices 620 can store data corresponding to the emission of electromagnetic radiation. To illustrate, the one or more data storage devices 620 can store data indicating the emission of electromagnetic radiation of one or more wavelengths for one or more periods of time by the emitter circuitry 616. The one or more data storage devices 620 can also store wavelengths of electromagnetic detected by the detector circuitry 618 during one or more periods of time. In various implementations, the one or more data storage devices 620 can store data indicating an intensity of electromagnetic radiation emitted by the emitter circuitry 616 and an intensity of electromagnetic radiation detected by the detector circuitry 618. The one or more data storage devices 620 can include random access memory. In one or more implementations, the one or more data storage devices 620 can include read only memory. In additional implementations, the one or more data storage devices 620 can include flash memory.

The processor circuitry 622 can include one or more processors to execute computer-readable instructions. The computer-readable instructions can be stored by the one or more data storage devices 620. In one or more implementations, computer-readable instructions executed by the processor circuitry 622 can be stored by cache memory. In various implementations, the processor circuitry 622 can include a floating-point unit. In one or more illustrative examples, the processor circuitry 622 can execute computer-readable instructions that can analyze electromagnetic radiation wavelength emission data, electromagnetic wavelength detection data, electromagnetic wavelength attenuation data, or one or more combinations thereof, to determine a level of anemia or a probability of anemia being present in an individual wearing the wearable device 600. For example, the processor circuitry 622 can determine an amount of attenuation of electromagnetic radiation for one or more wavelengths and determine blood-related information based on the amount of attenuation. The amount of attenuation of the electromagnetic radiation for the one or more wavelengths can corresponds to the presence or absence of hemoglobin metalloproteins. The amount of hemoglobin metalloproteins present in the blood of an individual can be used by the processor circuitry 622 to determine a level of anemia or a probability of anemia being present in the individual.

Further, the electronic components 614 can include communications circuitry 624. The communications circuitry 624 can include transmission circuitry to transmit data and receiver circuitry to receive data. The communications circuitry 624 can also include one or more antennas. The communications circuitry 624 can transmit and receive data according to one or more communications protocols. For example, the communications circuitry 624 can transmit and receive data according to a Bluetooth communications protocol. The communications circuitry 624 can also transmit and receive data according to a wireless local area network communications protocol, such as one or more Institute of Electrical and Electronics Engineers (IEEE) 1102.11 protocols. In various implementations, the communications circuitry 624 can transmit and receive data according to one or more wide area wireless communication standards, such as one or more cellular communication standards.

The electronic components 614 can include power circuitry 626 and one or more energy storage devices 628. The power circuitry 626 can control the supply of power to one or more of the electronic components 614 from the one or more energy storage devices 628. In various implementations, the power circuitry 626 can control charging of the one or more energy storage devices 628. The one or more energy storage devices 628 can include at least one battery. In one or more implementations, the one or more energy storage devices 628 can include at least one supercapacitor. In one or more illustrative examples, the one or more energy storage devices 628 can include a battery supplying from about 1 volt (V) to about 5 volts. Additionally, the one or more energy storage devices 628 can include a rechargeable battery, such as a lithium-ion battery.

The electronic components 614 can also include one or more external interfaces 630. The one or more external interfaces 630 can provide a connection between the wearable device 600 and one or more external devices. For example, the one or more external interfaces 630 can provide a connection between the wearable device 600 and a monitoring device that collects data from the wearable device 600. Additionally, the one or more external interfaces 630 can provide a connection between the wearable device 600 and a mobile computing device, such as a smartphone and/or a tablet computing device. In one or more illustrative examples, the one or more external interfaces 630 can include a universal serial bus (USB) interface. In one or more additional illustrative examples, the one or more external interfaces 630 can include a micro-USB interface. In further examples, the one or more external interfaces 630 can include a Lightning interface or a Thunderbolt interface.

Additionally, the electronic components 614 can include signal conditioning circuitry 632. The signal conditioning circuitry 632 can include at least one of one or more filters or one or more amplifiers to modify signals received by the detector circuitry 618 The signal conditioning circuitry 632 can filter data related to the detection of electromagnetic radiation that is not emitted by the emitter circuitry 616. For example, the signal conditioning circuitry 632 can filter data obtained from the detector circuitry 618 that corresponds to ambient light in an environment that includes the wearable device 600.

After the wearable device 600 is placed on a finger of an individual, the wearable device 600 can collect data from the emitter circuitry 616 and the detector circuitry 618. The processor circuitry 622 can execute computer-readable instructions to analyze the data and determine a level of anemia of the individual. The processor circuitry 622 can determine and that corresponds to the level of anemia and then activate the respective indicator to be displayed via one or more of the number of user interface components 608.

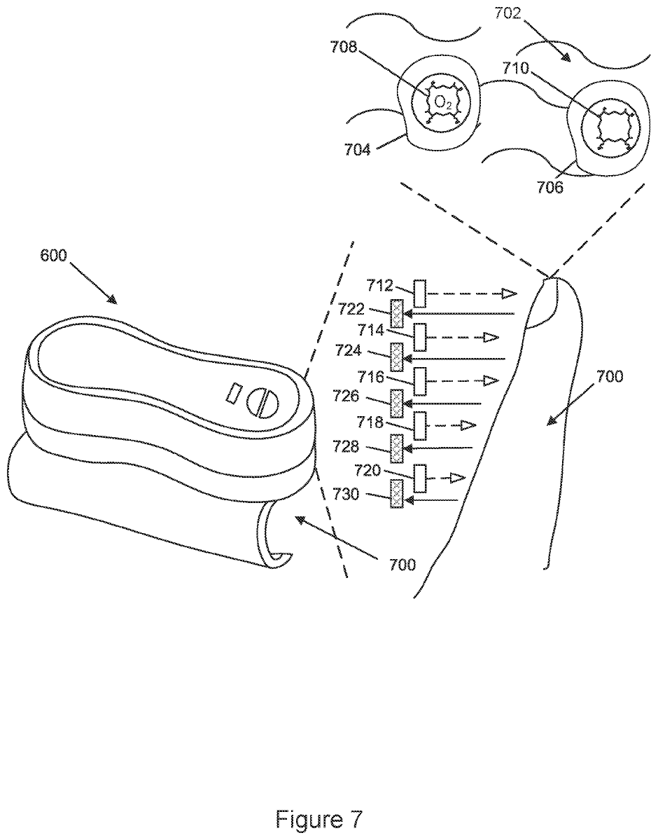

FIG. 7 illustrates another example wearable device 600 that can be placed on a finger 700 and that includes an array of emitters that each transmit electromagnetic radiation at different wavelengths and one or more detectors that detect electromagnetic radiation produced by the array of emitters with the emitters and detectors positioned on a same side of the wearable device. The illustrative example of FIG. 7 shows a portion of blood 702 that can be flowing through the finger 700. The blood 702 can include a first red blood cell 704 and a second red blood cell 706. The red blood cells 704, 706 can include a number of biomolecules. For example, the first red blood cell 704 can include a number of hemoglobin molecules, such as the first hemoglobin molecule 708, and the second red blood cell 706 can include an additional number of hemoglobin molecules, such as the second hemoglobin molecule 710. The first hemoglobin molecule 708 is bound to an oxygen molecule and the second hemoglobin molecule 710 is not bound to an oxygen molecule. Hemoglobin molecules that are bound to at least one oxygen molecule can be referred to herein as oxygenated hemoglobin. In addition, hemoglobin molecules that are not bound to an oxygen molecule can be referred to herein as de-oxygenated hemoglobin.

The wearable device 600 can include electronic components, such as emitter circuitry that includes a number of emitters 712, 714, 716, 718, 720 and detector circuitry that includes one or more detectors 722, 724, 726, 728, 730. The emitters 712, 714, 716, 718, 720 and the detectors 722, 724, 726, 728, 730 can be disposed in a body of the wearable device 600 such that the emitters 712, 714, 716, 718, 720 and detectors 722, 724, 726, 728, 730 are located on a same side of the finger 700 in one or more illustrative examples, the emitters 712, 714, 716, 718, 720 and the detectors 722, 724, 726, 728, 730 can be disposed on a single semiconductor chip. In one or more additional illustrative examples, the detectors 722, 724, 726, 728, 730 can be included in a single photodiode that detects a range of wavelengths emitted by the emitters 712, 714, 716, 718, 720, such as from about 500 nm to about 1400 nm or from about 600 nm to about 1000 nm.

The number of emitters 712, 714, 716, 718, 720 can include LEDs that emit different wavelengths of electromagnetic radiation and the one or more detectors 722, 724, 726, 728, 730 can include photodiodes that can detect the wavelengths of electromagnetic radiation emitted by the emitters. The emitters 712, 714, 716, 718, 720 can also emit electromagnetic radiation with one or more intensities. In addition, the detectors 722, 724, 726, 728, 730 can detect one or more intensities of electromagnetic radiation. In the illustrative example of FIG. 7, the electronic components can include a first emitter 712 that can be paired with a first detector 722, a second emitter 714 that can be paired with a second detector 724, a third emitter 716 that can be paired with a third detector 726, a fourth emitter 718 that can be paired with a fourth detector 728, and a fifth emitter 720 that can be paired with a fifth detector 730. Although the illustrative example of FIG. 7 indicates five different detectors 722, 724, 726, 728, 730, in additional implementations, the detector circuitry can include a different number of detectors. In an additional example, the detector circuitry can include a single detector that detects electromagnetic radiation emitted by the emitters 712, 714, 716, 718, 720. Further, in various implementations, the emitter circuitry can include more or fewer emitters to emit electromagnetic radiation at a number of different wavelengths.

The first emitter 712 can emit a first range of wavelengths of electromagnetic radiation, the second emitter 714 can emit a second range of wavelengths of electromagnetic radiation, the third emitter 716 can emit a third range of wavelengths of electromagnetic radiation, the fourth emitter 718 can emit a fourth range of wavelengths of electromagnetic radiation, and the fifth emitter 720 can emit a fifth range of wavelengths of electromagnetic radiation. In one or more illustrative examples, the first emitter 712 can emit wavelengths of electromagnetic radiation from about 610 nm to about 700 nm. In addition, the second emitter 714 can emit wavelengths of electromagnetic radiation from about 710 to about 770 nm. Further, the third emitter 716 can emit wavelengths of electromagnetic radiation from about 780 nm to about 840 nm. The fourth emitter 718 can emit wavelengths of electromagnetic radiation from about 810 nm to about 900 nm. The fifth emitter 720 can emit wavelengths of electromagnetic radiation from about 910 nm to about 990 nm.

The presence or absence of various molecules within the blood 702 can impact the attenuation of electromagnetic radiation transmitted from the emitters 712, 714, 716, 718, 720. For example, oxygenated hemoglobin can absorb electromagnetic radiation at a first set of wavelengths and de-oxygenated hemoglobin can absorb electromagnetic radiation at a second set of wavelengths that is different from the first set of wavelengths. By determining an amount of attenuation of various wavelengths of electromagnetic radiation emitted by the emitters 712, 714, 716, 718, 720 based on electromagnetic radiation detected by one or more of the detectors 722, 724, 726, 728, 730, the wearable device 600 can determine blood-related information for an individual.

In one or more illustrative examples, a protocol for the emission and detection of electromagnetic radiation by the wearable device 600 can be followed to determine information corresponding to the blood 702. For example, the first emitter 712 can emit first electromagnetic radiation having a first range of wavelengths for a first period of time and one or more of the detectors 722, 724, 726, 728, 730 can detect an amount of the first electromagnetic radiation that is not absorbed by one or more types of molecules included in the blood 702. After the first emitter 712 has completed emitting electromagnetic radiation, a pause can occur for a period of time and the second emitter 714 can emit second electromagnetic radiation having a second range of wavelengths for a second period of time after the pause. One or more of the detectors 722, 724, 726, 728, 730 can detect an amount of the second electromagnetic radiation that is not absorbed by one or more types of molecules included in the blood 702. An additional pause can follow the emission of the second electromagnetic radiation and after the additional pause, the third emitter 716 can emit third electromagnetic radiation having a third range of wavelengths for a third period of time. One or more of the detectors 722, 724, 726, 728, 730 can detect an amount of the third electromagnetic radiation that is not absorbed by one or more types of molecules in the blood 702. Further, following a pause after the third period of time, the fourth emitter 718 can emit fourth electromagnetic radiation having a fourth range of wavelengths for a fourth period of time. In various implementations, one or more of the detectors 722, 724, 726, 728, 730 can detect an amount of the fourth electromagnetic radiation that is not absorbed by one or more types of molecules included in the blood 702. Following a pause after the fourth period of time, the fifth emitter 720 can emit fifth electromagnetic radiation having a fifth range of wavelengths for a fifth period of time. One or more of the detectors 722, 724, 726, 728, 730 can detect an amount of the fifth electromagnetic radiation that is not absorbed by one or more types of molecules included in the blood 702.

After implementing a protocol regarding the emission of electromagnetic radiation by the emitters 712, 714, 716, 718, 720 and detection of electromagnetic radiation by one or more of the detectors 722, 724, 726, 728, 730, the amount of attenuation of each of the ranges of electromagnetic radiation can be determined by the wearable device 600. The amount of attenuation of each of the ranges of electromagnetic radiation can indicate the presence or absence of one or more biomolecules in the blood 702. By analyzing the respective amounts of the one or more biomolecules in the blood 702, a level of anemia can be determined for an individual. For example, an amount of oxygenated hemoglobin, an amount of de-oxygenated hemoglobin, and a packed cell volume of the blood 702 can be determined based on the attenuation of electromagnetic radiation emitted by the emitters 712, 714, 716, 718, 720. The amount of oxygenated hemoglobin, an amount of de-oxygenated hemoglobin, and a packed cell volume of the blood 702 can then be used to determine a level of anemia of an individual in which the blood 702 is flowing.

In one or more illustrative examples, the attenuation of electromagnetic radiation determined based on data produced by one or more of the emitters 712, 714, 716, 718, 720 and data produced by one or more of the detectors 722, 724, 726, 728, 730 can be used to determine a level of anemia or a probability of anemia being present in an individual. For example, an amount of attenuation of electromagnetic radiation at wavelengths from about 850 nm to about 1000 nm, such as from about 910 nm to about 990 nm, can indicate an amount of oxygenated hemoglobin present in the blood of individuals and an amount of attenuation of electromagnetic radiation at wavelengths from about 600 nm to about 750 nm, such as from about 630 nm to about 690 nm or from about 710 nm to about 790 nm, can indicate an amount of deoxygenated hemoglobin present in the blood of individuals. One or more correlations can be made between the amount of oxygenated hemoglobin and/or the amount of deoxygenated hemoglobin present in the blood of individuals with a level of anemia or having at least a threshold probability of being diagnosed with anemia. In various examples, the correlations can be made using previously obtained oxygenated hemoglobin levels and/or deoxygenated hemoglobin levels of individuals for which a respective level of anemia has been determined. To illustrate, a first level of oxygenated hemoglobin and/or a first level of deoxygenated hemoglobin can be correlated to a first level of anemia or a first range of probabilities of anemia being present in individuals based on information obtained from individuals previously diagnosed with anemia and a second level of oxygenated hemoglobin and/or a second level of deoxygenated hemoglobin can be correlated to a second level of anemia or a second range of probabilities of anemia being present in individuals based on information obtained from individuals previously diagnosed with anemia. The amounts of deoxygenated hemoglobin and/or oxygenated hemoglobin determined based on data obtained from one or more of the emitters 712, 714, 716, 718, 720 and one or more of the detectors 722, 724, 726, 728, 730 can then be compared against the predetermined correlations to determine a level of anemia or a probability of anemia being present in an individual wearing the wearable device 600.

In one or more implementations, the correlations between amounts of deoxygenated hemoglobin and oxygenated hemoglobin that correspond to levels of anemia or probabilities of being diagnosed with anemia can be expressed as one or more ratios. In various examples, the amount of attenuation of electromagnetic radiation of one or more wavelengths can be expressed as an amount of current produced by the one or more detectors 722, 724, 726, 728, 730 based on an amount of electromagnetic radiation incident on the one or more detectors 722, 724, 726, 728, 730 or an amount of voltage derived from the current produced by the one or more detectors 722, 724, 726, 728, 730. In one or more illustrative examples, the correlations used to determine a level of anemia can be based on at least one of a direct current (DC) component or an alternating current (AC) component of the current derived from the one or more detectors 722, 724, 726, 728, 730 based on electromagnetic radiation sensed by the one or more detectors 722, 724, 726, 728, 730. Current and/or voltage readings can also be determined in response to the emission of electromagnetic radiation of the emitters 712, 714, 716, 718, 720. In these situations, ratios of current and/or ratios of voltage related to the emission and the detection of electromagnetic radiation by the wearable device can be used to determine a level of anemia or a probability of anemia being present based on previously determined correlations between respective levels of anemia or respective probabilities of anemia being present and one or more current ratios and/or voltage ratios. In additional implementations, intensity of electromagnetic radiation emitted by one or more of the emitters 712, 714, 716, 718, 720 and intensity of electromagnetic radiation detected by one or more of the detectors 722, 724, 726, 728, 730 can also be correlated to levels of anemia. For example, ratios of intensity of emitted electromagnetic radiation and intensity of detected electromagnetic radiation can be used to determine a level of anemia or a probability of anemia being present based on previously determined correlations between levels of anemia or probabilities of anemia being present and ratios of intensity of emitted electromagnetic radiation with respect to intensity of detected electromagnetic radiation.

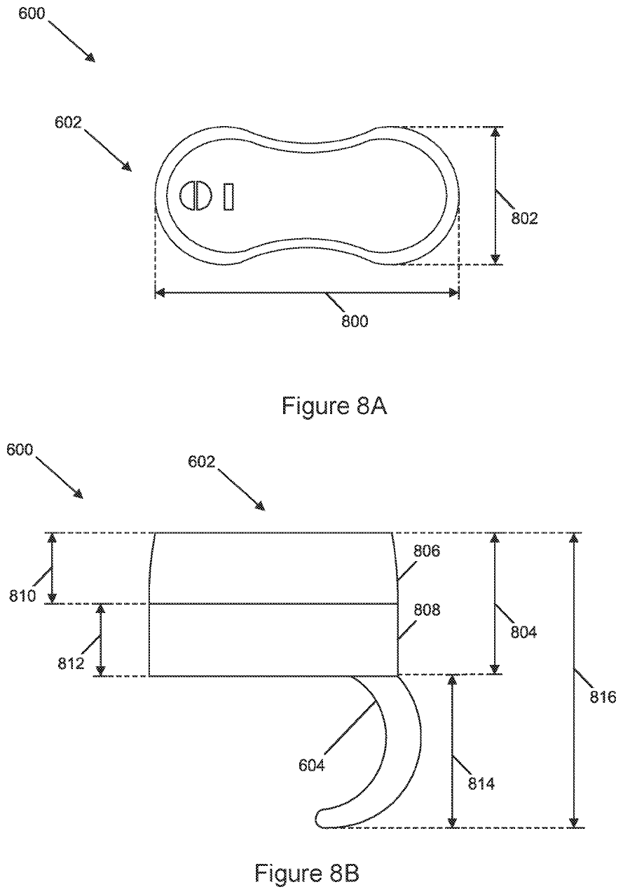

FIG. 8A illustrates a top view of an example of an additional wearable device 600 that generates data that can be used to determine a probability of anemia being present in an individual. The body 602 of the wearable device 600 can have a length 800 and a width 802. The length 800 of the body 602 can be from about 3 cm to about 12 cm, from about 4 cm to about 10 cm, or from about 5 cm to about 8 cm. In addition, the width 802 of the body 602 can be from about 0.5 cm to about 5 cm, from about 1 cm to about 4 cm, or from about 2 cm to about 3 cm.

FIG. 8B illustrates a side view of an example of an additional wearable device 600 that generates data that can be used to determine a probability of anemia being present in an individual. The body 602 of the wearable device 600 can have a height 804. The height 804 can be from about 0.5 cm to about 4 cm or from about 1 cm to about 3 cm. The body 602 can be comprised of a first portion 806 and a second portion 808. In one or more examples, the first portion 806 can be separated from the second portion 808. Additionally, the second portion 808 can be coupled to the clamp member 604. The first portion 806 can have a first height 810 and the second portion 808 can have a second height 812. The first height 810 can be from about 0.4 cm to about 2 cm or from about 0.6 cm to about 1.3 cm. The second height 812 can be from about 0.3 cm to about 2 cm or from about 0.5 cm to about 1.2 cm.

The clamp member 604 can have a height 814. The height 814 of the clamp member 604 can be from about 0.5 cm to about 3 cm, from about 0.8 cm to about 2 cm, or from about 1 cm to about 1.6 cm. Although not shown in the illustrative example of FIG. 8B, the clamp member 604 can also have a length that is disposed along the length 800 of the wearable device 600. In one or more examples, the length of the clamp member 604 can be the same as the length of the length 800. In one or more additional examples, the length of the clamp member 604 can be less than the length 800. In one or more illustrative examples, the length of the clamp member 604 can be from about 2 cm to about 10 cm, from about 3 cm to about 8 cm, or from about 4 cm to about 6 cm. The wearable device 600 can have an overall height 816 that includes the height 804 of the body 602 and the height 814 of the clamp member 604. The overall height 816 can be from about 1 cm to about 8 cm or from about 2 cm to about 5 cm.

The dimensions and curvature of the clamp member 604 can be configured such that the wearable device 600 can be coupled to a finger of an individual. In various implementations, the dimensions of the wearable device 600 can correspond to different sizes of finger of individuals. For example, one or more first implementations of the wearable device 600 can have dimensions that correspond to those of a finger of a child, while one or more second implementations of the wearable device 600 can have dimensions that correspond to those of a finger of an adult. In one or more additional implementations, the wearable device 600 can have dimensions that correspond to those of a toe of a child or to a toe of an adult.

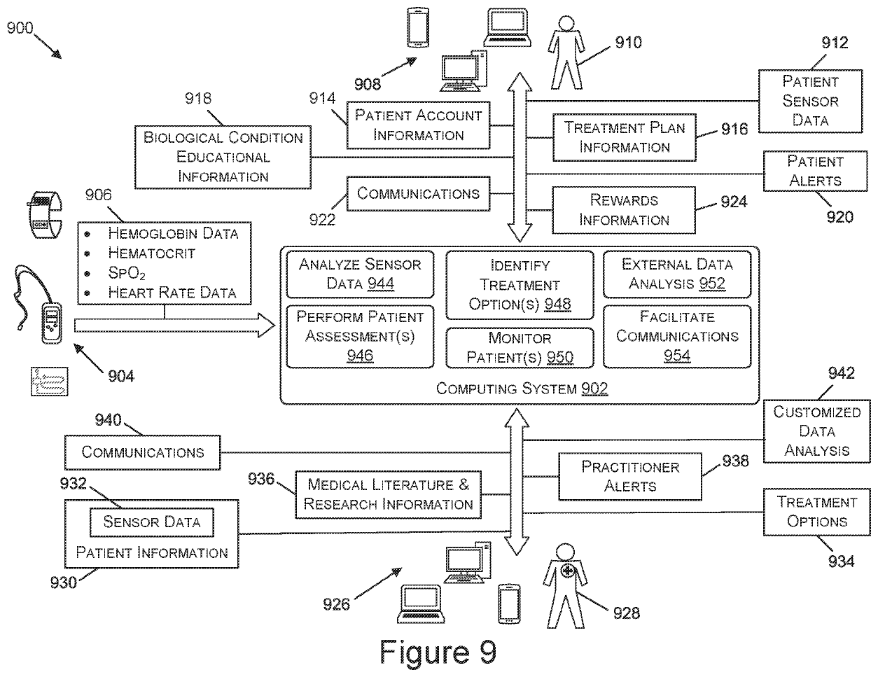

FIG. 9 illustrates an example environment 900 to facilitate communication between individuals and healthcare providers regarding information corresponding to the presence of one or more biological conditions in at least a portion of the individuals. The environment 900 can include a computing system 902 that can obtain blood-related data of individuals, analyze the blood-related data, and provide communications related to the blood-related data. In various implementations, the computing system 902 can analyze the blood-related data to determine a probability that one or more individuals are anemic. In these scenarios, the computing system 902 can provide communications to the one or more individuals and/or to healthcare practitioners corresponding to potentially anemic conditions with respect to the one or more individuals. In one or more implementations, the computing system 902 can be a part of the health data system 102 of FIG. 1 or implemented in conjunction with the health data system 102.

The environment 900 can include one or more data capture devices 904 that can obtain blood-related data of one or more individuals. The one or more data capture devices 904 can include one or more wearable devices that can be worn on a portion of a body of an individual. For example, the one or more data capture devices 904 can include one or more wearable devices that can be worn on a wrist of an individual. Additionally, the one or more data capture devices 904 can include one or more wearable devices that can be worn on a finger of an individual. In one or more illustrative examples, the one or more data capture devices 904 can include the wearable device 200 described with respect to FIGS. 2-5 and/or the wearable device 600 described with respect to FIGS. 6-8.

The one or more data capture devices 904 can also include one or more monitoring devices that can include a sensor portion and a hand-held portion. The sensor portion can obtain data produced by one or more sensors included in the sensor portion. The sensor portion can be coupled to or worn on a body of an individual. To illustrate, the sensor portion can be coupled to or worn on a finger, a wrist, an arm, a foot, and/or an ankle. The hand-held portion can include one or more display devices for displaying information corresponding to the data obtained by the sensor portion. The hand-held portion can also include one or more computer components to analyze, process, and/or display the information obtained from the sensor portion. In various examples, the hand-held portion can include at least one of one or more input devices or one or more communication devices.

Further, the one or more data capture devices 904 can include one or more lab-on-a-chip devices that can analyze a blood sample or a tissue sample of an individual. In one or more examples, the one or more lab-on-a-chip devices can be a component of a larger device. For example, the one or more lab-on-a-chip devices can be included in a mobile computing device, such as a smartphone. The one or more lab-on-a-chip devices can also be part of a handheld monitoring device.

The one or more data capture devices 904 can be in communication with the computing system 902. At least a portion of the one or more data capture devices 904 can be in direct communication with the computing system 902 via one or more communication interfaces of the one or more data capture devices 904. For example, the one or more data capture devices 904 can be in communication with the computing system 902 via one or more wired communication networks, one or more wireless communication networks, one or more local area communication networks, one or more wide area communication networks, or one or more combinations thereof. Additionally, the one or more data capture devices 904 can be indirectly in communication with the computing system 902. In these situations, the one or more data capture devices 904 can be in direct communication with one or more intermediate devices. In various examples, the one or more intermediate devices can be in direct communication with the computing system 902. In one or more illustrative examples, one or more data capture devices 904 can be in communication with an intermediate device, such as a mobile computing device, using a Bluetooth communication interface and the intermediate device can be in communication with the computing system 902 via a wired communication interface, a cellular communication interface, and/or a local wireless network communication interface.

The one or more data capture devices 904 can obtain health data 906 from one or more individuals and send the health data 906 to the computing system 902. The health data 906 can include blood-related data. For example, the health data 906 can include information related to hemoglobin biomolecules of individuals. To illustrate, the health data 906 can include information corresponding to a number of oxygenated hemoglobin molecules included in a volume of blood of an individual. Additionally, the health data 906 can include information corresponding to a number of deoxygenated hemoglobin molecules included in a volume of blood of an individual. Further, the health data 906 can include information corresponding to total hemoglobin levels included in a volume of blood of an individual. The health data 906 can also include information related to hematocrit levels of an individual. In various examples, the hematocrit levels can be determined based on the hemoglobin data obtained by the one or more data capture devices 904. The health data 906 can include additional blood-related information, such as mean corpuscular volume values, mean corpuscular hemoglobin concentration, red cell distribution width, or one or more combinations thereof. Further, the health data 906 can include additional information, such as heart rate data and peripheral capillary oxygen saturation (SpO₂) levels.

In one or more implementations, the health data 906 can include raw sensor data generated by sensing components of the one or more data capture devices 904. The raw sensor data can indicate wavelengths of electromagnetic radiation transmitted by one or more emitting devices of the one or more data capture devices 904. The raw sensor data can also include intensity levels of electromagnetic radiation transmitted by one or more emitting devices. In addition, the raw sensor data can indicate intensity values and/or wavelengths of electromagnetic radiation detected by one or more detecting devices of the one or more data capture devices 904. Further, the health data 906 can include timing data indicating times of emission and times of detection of one or more wavelengths of electromagnetic radiation. In one or more illustrative examples, the health data 906 can include attenuation values and/or scattering values with respect to electromagnetic radiation emitted and/or detected by components of the one or more data capture devices 904. In various examples, the health data 906 can be derived from the raw sensor data generated by the one or more sensor devices.

The environment 900 can also include one or more patient computing devices 908 that can be operated by one or more patients 910. The one or more patient computing devices 908 can include one or more mobile computing devices, one or more smart phones, one or more table computing devices, one or more laptop computing devices, one or more desktop computing devices, one or more additional computing devices, or one or more combinations thereof. The one or more patient computing devices 908 can be in communication with the computing system 902. The computing system 902 can facilitate the communication of various types of information between the one or more patient computing devices 908 and the computing system 902. In various examples, the one or more patient computing devices 908 can execute a mobile device application and/or a browser application that can process and display information obtained by the one or more patient computing devices 908.

In one or more illustrative examples, the computing system 902 can provide patient sensor data 912 to the one or more patient computing devices 908. The patient sensor data 912 can include data obtained from and/or derived from the health data 906. For example, the patient sensor data 912 can include blood-related information of a patient 910, SpO2 information of a patient 910, heart rate data of a patient 910, or one or more combinations thereof. The patient sensor data 912 can also include an indication of a probability of anemia being present in a patient 910. To illustrate, the patient sensor data 912 can indicate a level of anemia for a patient 910 that corresponds to a probability that the patient 910 is anemic. In one or more implementations, the patient sensor data 912 can indicate at least portions of the health data 906 of a patient 910 over a period of time, such as a number of hours, a number of days, a number of weeks, a number of months. Further, the patient sensor data 912 can indicate levels of anemia of a patient 910 over a period of time.

The computing system 902 and the one or more patient computing devices 908 can also communicate patient account information 914. The patient account information 914 can include information related to an account of a patient 910 with a service provider that provides health related information to individuals. In one or more illustrative examples, the service provider can provide information related to blood disorders, such as anemia, to individuals. In various examples, the service provider can implement at least a portion of the computing system 902. In one or more implementations, the patient account information 914 can include one or more identifiers of a patient 910, such as one or more names, one or more login identifiers, one or more passwords, one or more identification numbers, one or more account numbers, or one or more combinations thereof. The patient account information 914 can also include financial information related to one or more patients 910, such as financial account information and/or payment transactions. The payment transactions can be in relation to one or more services of a service provider that provides health related information to individuals, such as anemia diagnosis and treatment services.

Additionally, the computing system 902 and the one or more patient computing devices 908 can communicate treatment plan information 916. The treatment plan information 916 can include one or more treatments that a patient 910 can obtain with respect to one or more biological conditions. In various examples, the treatment plan information 916 can correspond to one or more treatments for blood-related disorders, such as anemia. In one or more illustrative examples, the treatment plan information 916 can include one or more activities that a patient 910 can perform with respect to the treatment of a biological condition. Additionally, the treatment plan information 916 can include one or more medications, one or more therapeutics, and/or one or more nutritional supplements that a patient 910 can consume with respect to the treatment of a biological condition. Further, the treatment plan information 916 can include nutritional information that can be used with respect to the treatment of a biological condition. The treatment plan information 916 can also include information provided by a patient 910 related to following a treatment plan for a biological condition. For example, the treatment plan information 916 can correspond to a journal or a log of activities taken by a patient 910 with respect to a treatment plan. To illustrate, the treatment plan information 916 can indicate timing and/or frequency of activities performed by a patient 910 with respect to a treatment plan. In one or more implementations, the treatment plan information 916 can indicate at least one of physical activity performed by a patient 910, a time that the physical activity was performed by the patient 910, food and/or beverages consumed by the patient 910, or a time that the food and/or beverages were consumed by the patient 910. In further implementations, the treatment plan information 916 can indicate at least one of one or more medications, one or more therapeutics, or one or more nutritional supplements consumed by a patient 910 in addition to a time of consumption for the one or more medications, the one or more therapeutics, and/or the one or more nutritional supplements.

Further, the computing system 902 and the one or more patient computing devices 908 can communicate biological condition educational information 918. The biological condition educational information 918 can include information indicating symptoms of a biological condition, treatments for a biological condition, prognosis for a biological condition, causes of a biological condition, discussion forums related to a biological condition, or one or more combinations thereof. The biological condition educational information 918 can include one or more webpages, links to one or more websites, journal articles, research papers, textbooks, or one or more combinations thereof.

The computing system 902 can also provide patient alerts 920 to the one or more patient computing devices 908. The patient alerts 920 can be related to the health data 906 obtained by the computing system 902 from the one or more data capture devices 904. For example, the patient alerts 920 can indicate that one or more measures of a biological conditions are outside of a threshold range. To illustrate, the patient alerts 920 can indicate that a probability of a patient 910 being anemic has exceeded a threshold probability. Additionally, the patient alerts 920 can indicate that SpO₂ levels of a patient 910 have fallen below a threshold level and/or that a pulse rate of a patient 910 is outside of a specified range. The patient alerts 920 can also include reminders with regard to treatment of a biological condition. In one or more illustrative examples, the patient alerts 920 can indicate a time for a patient 910 to consume at least one of a therapeutic, a nutritional supplement, or a medication. Further, the patient alerts 920 can include reminders for a patient 910 to perform one or more physical exercises or to consume one or more foods and/or beverages.

In various implementations, the computing system 902 can transmit and/or receive communications 922 with respect to the one or more patient computing devices 908. The communications 922 can include emails, text messages, video messages, calls, or one or more combinations thereof. The communications 922 can also include voice calls and/or video calls. In one or more examples, the communications 922 can be between the one or more patient computing devices 908 and one or more additional computing devices, such as one or more computing devices of at least one healthcare practitioner.

In one or more implementations, the computing system 902 can communicate rewards information 924 to the one or more patient computing devices 908. The rewards information 924 can indicate rewards that a patient 910 can accumulate by following one or more treatment protocols for a biological condition. In one or more illustrative examples, a patient 910 that is anemic can accumulate rewards by consuming an iron supplement at one or more specified times daily. The rewards information 924 can indicate virtual rewards and/or physical rewards that can be accumulated by a patient 910.

The environment 900 can also include one or more practitioner computing devices 926 that can be operated by one or more healthcare practitioners 928. The one or more practitioner computing devices 926 can include one or more mobile computing devices, one or more smart phones, one or more table computing devices, one or more laptop computing devices, one or more desktop computing devices, one or more additional computing devices, or one or more combinations thereof. The one or more practitioner computing devices 926 can be in communication with the computing system 902. The computing system 902 can facilitate the communication of various types of information between the one or more practitioner computing devices 926 and the computing system 902. In various examples, the one or more practitioner computing devices 926 can execute a mobile device application and/or a browser application that can process and display information obtained from the computing system 902. The one or more healthcare practitioners 928 can include a doctor, a nurse, a technician, a practitioner assistant, or another healthcare worker that is involved in providing treatment to one or more patients, including the patient 910.

The computing system 902 can communicate patient information 930 to the one or more practitioner computing devices 926. The patient information 930 can include health related information for one or more patients treated by the one or more healthcare practitioners 928. For example, the patient information 930 can include at least one of one or more medical records, one or more laboratory test results, one or more medical images, one or more healthcare practitioner notes, one or more healthcare practitioner observations, vital signs information, data obtained from one or more medical devices, or one or more combinations thereof. In one or more illustrative examples, the patient information 930 can include sensor data 932. The sensor data 932 can include data obtained from the one or more data capture devices 904. In various implementations, a healthcare practitioner 928 can access at least a portion of the sensor data 932 for a patient 910. To illustrate, the healthcare practitioner 928 can access blood-related information associated with the patient 910, such as hemoglobin data, hematocrit data, and/or red blood cell count. The patient information 930 can also include anemia related data, such as diagnostic health information that can be used by the healthcare practitioner 928 to make a diagnosis for the patient 910 with respect to anemia. In one or more implementations, the patient information 930 can include data indicating levels of anemia of the patient 910. The levels of anemia can indicate a probability that the patient 910 is anemic and/or a severity of anemia with respect to the patient 910. Additionally, the patient information 930 can indicate at least one of sensor data 932, blood-related information, or anemia-related information of the patient 910 over a period of time.

The computing system 902 can also provide one or more treatment options 934 to the one or more practitioner computing devices 926. The one or more treatment options 934 can include recommendations for treating one or more biological conditions of one or more patients of at least one healthcare practitioner 928. In various examples, the one or more treatment options 934 can include one or more treatment protocols for one or more biological conditions. For example, the one or more treatment options 934 can indicate a pharmaceutical product that can be used to treat a biological condition, a frequency for the pharmaceutical product to be consumed, and/or a duration for the pharmaceutical product to be consumed. In addition, the treatment options 934 can indicate at least one of nutritional information, one or more nutritional supplements to be consumed, one or more physical activity regimens, one or more meal plans, one or more surgical procedures, or one or more medical devices to be used with respect to the treatment of one or more biological conditions. In one or more implementations, a healthcare practitioner 928 can provide at least a portion of the one or more treatment options 934 to a patient 910 as part of the treatment plan information 916.

Further, the computing system 902 can provide medical literature and research information 936 to the one or more practitioner computing devices 926. The medical literature and research information 936 can include information corresponding to one or more biological conditions. For example, the medical literature and research information 936 can include symptoms of one or more biological conditions, treatments for one or more biological conditions, articles related to one or more biological conditions, clinical trials information, textbooks, seminars, classes, videos, audio information, or one or more combinations thereof. In various implementations, the medical literature and research information 936 can be accessed via one or more websites. In one or more illustrative examples, a healthcare practitioner 928 can request at least a portion of the medical literature and research information 936 using one or more search requests that can include one or more keywords. To illustrate, the computing system 902 can send medical literature and research information 936 to the one or more practitioner computing devices 926 in response to a request for information related to the diagnosis and/or treatment of anemia.

In addition, the computing system 902 can provide practitioner alerts 938 to the one or more practitioner computing devices 926. The practitioner alerts 938 can include information that corresponds to one or more patients being treated by one or more healthcare practitioners 928. For example, the practitioner alerts 938 can indicate metrics related to the health of individuals. The metrics can correspond to vital signs information of individuals, blood-related information of individuals, diagnostic information of individuals, laboratory test results of individuals, imaging data of individuals, or one or more combinations thereof. In various examples, the practitioner alerts 938 can indicate that health-related measures for individuals are outside of one or more threshold ranges. To illustrate, the practitioner alerts 938 can indicate that hemoglobin levels of a patient 910 are below one or more threshold levels. The practitioner alerts 938 can also indicate that hematocrit levels of a patient 910 are outside of one or more threshold levels. Further, the practitioner alerts 938 can indicate that a patient 910 is not following a treatment protocol for a biological condition. In one or more implementations, the practitioner alerts 938 can indicate that a patient 910 has not consumed a medication or nutritional supplement within a period of time, that a patient 910 has not performed one or more physical activities within a period of time, that a patient 910 has not gone to an appointment with a healthcare provider, and/or that a patient 910 has not received treatment for a biological condition.

The computing system 902 can also cause communications 940 to be exchanged between the one or more practitioner computing devices 926 and one or more additional computing devices, such as the one or more patient computing devices 908. The communications 940 can include emails, text messages, video messages, calls, or one or more combinations thereof. The communications 940 can also include voice calls and/or video calls. In one or more illustrative examples, the communications 940 can be related to the treatment of a patient 910 by a healthcare practitioner 928 for a biological condition.

In one or more implementations, the computing system 902 can provide customized data analysis 942 to the one or more practitioner computing devices 926. The customized data analysis 942 can include an analysis of health-related information of individuals, where the analysis is requested by one or more healthcare practitioners 928 using a respective practitioner computing device 926. In various examples, the customized data analysis 942 can correspond to an analysis by the computing system 902 with respect to health-related information of one or more patients 910 being treated by the one or more healthcare practitioners 928. The customized data analysis 942 can indicate one or more characteristics of individuals in which a biological condition is present. In one or more illustrative examples, the customized data analysis 942 can indicate blood-related information for a number of individuals being treated for anemia. For example, the customized data analysis 942 can indicate hemoglobin levels and/or hematocrit levels for at least a subset of patients 910 being treated by the one or more healthcare practitioners 928 for anemia. Additionally, the customized data analysis 942 can indicate blood-related information for a number of individuals in a geographic location. To illustrate, the customized data analysis 942 can indicate blood-related information of individuals located in a specified country, state, province, municipality, or other geographic region.

The computing system 902 can perform a number of functions to provide one or more services to at least one of the one or more patients 910 or the one or more healthcare practitioners 928. For example, the computing system 902 can perform sensor data analysis at operation 944. The sensor data analysis can include analyzing information obtained from the one or more data capture devices 904. To illustrate, the computing system 902 can analyze the health data 906 obtained from the one or more data capture devices 904. In various implementations, the sensor data analysis can include determining trends in health data 906 of patients 910 over a period of time. The sensor data analysis can also include analyzing the data obtained from the one or more data capture devices 904 with respect to one or more thresholds and/or one or more ranges. In one or more implementations, the sensor data analysis can include determining that at least a portion of the health data 906 for one or more individuals is outside of one or more specified ranges. In one or more illustrative examples, the sensor data analysis can include determining that heart rate data of an individual has exceeded a threshold heartrate for a threshold period of time. In one or more additional illustrative examples, the sensor data analysis can include determining that hemoglobin levels and/or hematocrit levels for an individual are below one or more threshold levels for hemoglobin levels and/or below one or more threshold levels for hematocrit.

In addition, the sensor data analysis at operation 944 can include analyzing raw data obtained from the one or more data capture devices 904. For example, the sensor data analysis can include determining a measure of a biological condition based on raw data obtained from the one or more data capture devices 904. To illustrate, the sensor data analysis can include analyzing at least one of electromagnetic radiation transmittance data, electromagnetic radiation intensity data, or electromagnetic radiation attenuation data obtained from the one or more data capture devices 904 to determine hemoglobin levels and/or hematocrit levels of individuals. Further, the sensor data analysis can include analyzing at least one of electromagnetic radiation transmittance data, electromagnetic radiation intensity data, or electromagnetic radiation attenuation data obtained from the one or more data capture devices 904 to determine peripheral capillary oxygen saturation levels and/or heart rate levels of one or more individuals.

The computing system 902 can also perform one or more patient assessments at operation 946. Performing a patient assessment at operation 946 can include determining that a biological condition is present in an individual. In one or more illustrative examples, the patient assessments can be based at least partly on diagnostic test results, laboratory test results, genetic information, medical records, imaging data, data obtained from the one or more data capture devices 904, or one or more combinations thereof. In one or more implementations, the patient assessments can be determined based on information provided by one or more patients 910 to the computing system 902. For example, the computing system 902 can provide a patient portal that obtains information from patients 910, such as symptoms, vital sign information, image data, and so forth. In this way, a patient assessment can be determined using information provided directly to the computing system 902 from a patient 910 via a respective patient computing device 908.

In various examples, a patient assessment can be performed at operation 946 based on the sensor data analysis performed at operation 944. For example, the computing system 902 can perform a patient assessment to determine that an individual has at least a threshold probability of being anemic based on an analysis of hemoglobin and/or hematocrit levels determined by the sensor data analysis. In one or more additional examples, the computing system 902 can perform a patient assessment to determine that individual has less than a threshold probability of being anemic based on an analysis of hemoglobin and/or hematocrit levels determined by the sensor data analysis.

In one or more implementations, the patient assessments can be performed at operation 946 using one or more computational models. The one or more computational models can be generated by the computing system 902 using one or more machine learning techniques. In one or more illustrative examples, the one or more computational models can include one or more neural networks. In various implementations, the one or more computational models can include one or more multilayer perceptron networks. The one or more computational models can be generated based on training data and testing data that is accessible to the computing system 902. The training data and the testing data can indicate characteristics of individuals that have previously been diagnosed with a biological condition. The one or more computational models implemented to perform the patient assessments can be generated by analyzing the training data to determine relationships between characteristics of individuals diagnosed with a biological condition. A computational model that has been trained and tested with respect to a biological condition can be used to perform one or more patient assessments by applying the computational model with respect to data associated with a patient 910. The data used to perform a patient assessment at operation 946 for a patient 910 with respect to a biological condition can correspond to one or more variables and/or one or more nodes included in a computational model, where the data can be used by the computational model to determine a probability of an individual having the biological condition.

Performing one or more patient assessments at operation 946 can also include determining one or more recommendations for one or more additional procedures to be performed with respect to one or more individuals that have at least a threshold probability of being diagnosed with a biological condition. For example, the computing system 902 can determine that an individual has at least a threshold probability of being diagnosed with anemia of chronic disease (ACD). In these situations, the computing system 902 can determine one or more diagnostic tests, one or more laboratory tests, one or more imaging procedures, one or more medical procedures, one or more clinical procedures, or one or more combinations thereof, to be performed with respect to the individual to identify a potential chronic disease from which the individual is suffering. Additionally, the computing system 902 can determine one or more imaging procedures that can be used to identify whether an individual that has at least a threshold probability of being anemic is suffering from internal bleeding.

In one or more illustrative examples, performing patient assessments at operation 946 can include analyzing sensor data to determine characteristics of blood of individuals. The computing system 902 can use the characteristics of blood of individuals determined from the sensor data to determine one or more types of anemia that individuals can have. For example, data corresponding to transmittance of electromagnetic radiation, intensity of electromagnetic radiation, and/or attenuation of electromagnetic radiation received from the one or more data capture devices 904 can be analyzed by the computing system 902 and used to determine at least one of mean corpuscular volume (MCV) of blood of individuals, levels of oxygenated hemoglobin, levels of deoxygenated hemoglobin, total hemoglobin levels, red cell distribution width (RDW), or mean corpuscular hemoglobin concentration (MCHC). In addition to blood characteristics of an individual, additional characteristics of the individual can also be considered in determining whether the individual may be diagnosed with anemia. To illustrate, at least one of age, gender, pregnancy status, or menstruation status can also be taken into consideration by the computing system 902 in determining whether the individual is anemic and, in situations where the individual is anemic, in determining a type of anemia of the individual.

In various examples, the computing system 902 can determine a level of MCV for a patient 910 as part of determining a patient assessment at operation 946. Based on the level of MCV of the patient 910, the computing system 902 can then determine additional blood characteristics of the patient 910. For example, in situations where the MCV levels of the patient 910 are within at least a first range of values, the computing system 902 can determine a first additional characteristic of the blood of the patient 910. Additionally, in scenarios where the MCV levels of the patient 910 are within at least a second range of values, the computing system 902 can determine a second additional characteristic of the blood of the patient 910. In one or more illustrative examples, the computing system 902 can determine MCHC values based on MCV values being within a range from about 80 femtoliters (fL) to about 100 fL. Additionally, the computing system 902 can determine total hemoglobin levels based on MCV values being within a range of about 80 fL to about 100 fL. Further, the computing system 902 can determine total hemoglobin levels based on MCV values being less than about 80 fL or based on MCV values being greater than about 100 fL.

In situations where the computing system 902 determines that the MCV levels of the patient 910 are from about 80 fL to about 100 fL and that MCHC levels of the patient 910 are less than 32 grams/deciliter (g/dL), the computing system 902 can then determine total hemoglobin levels for the patient 910 and also RDW levels for the patient 910. In scenarios, where total hemoglobin levels are less than 10 g/dL and RDW levels are from about 11.6% to about 14.6%, then the computing system 902 can determine that the patient 910 has at least a threshold probability of being diagnosed with severe thalassemia. Additionally, in implementations where total hemoglobin levels are less than 10 g/dl and RDW levels are greater than about 14.6%, the computing system 902 can determine that the patient 910 has at least a threshold probability of being diagnosed with severe iron deficiency anemia. Further, in situations where the total hemoglobin levels of the patient 910 are from about 10 g/dL to about 12 g/dL and RDW levels are from about 11.6% to about 14.6%, the computing system 902 can determine that the patient 910 has at least a threshold probability of being diagnosed with moderate thalassemia. Also, where total hemoglobin levels of the patient 910 are from about 10 g/dL to about 12 g/dL and RDW levels are greater than about 14.6%, the computing system 902 can determine that the patient 910 has at least a threshold probability of being diagnosed with moderate iron deficiency anemia.

In one or more implementations where the computing system 902 determines that the MCV levels of the patient 910 are from about 80 fL to about 100 fL and the MCHC levels of the patient 910 are from about 32 g/dL to about 36 g/dL, the computing system 902 can then determine total hemoglobin levels of the patient 910 to determine one or more biological conditions associated with the patient 910. For example, the computing system 902 can determine that the patient 910 has MCHC levels from about 32 g/dL to about 36 g/dL and total hemoglobin levels less than about 10 g/dL and determine that the patient 910 has at least a threshold probability of being diagnosed with severe anemia of renal disease. In one or more additional examples, the computing system 902 can determine that the patient 910 has MCHC levels from about 32 g/dL to about 36 g/dL and total hemoglobin levels from about 10 g/dL to about 12 g/dL and determine that the patient 910 has at least a threshold probability of being diagnosed with moderate anemia of renal disease.

In one or more examples where the MCV levels of the patient 910 are less than about 80 fL, the computing system 902 can also determine that the total hemoglobin levels of the patient 910 are less than about 10 g/dL and then determine that the patient 910 has at least a threshold probability of being diagnosed with severe anemia of chronic disease. In addition, the computing system 902 can determine that MCV levels of the patient 910 are less than about 80 fL and that the total hemoglobin levels of the patient 910 are from about 10 g/dL to about 12 g/dL to determine that the patient has at least a threshold probability of being diagnosed with moderate anemia of chronic disease.

In further implementations, the computing system 902 can determine that MCV levels of the patient 910 are greater than about 80 f and that the total hemoglobin levels of the patient 910 are less than about 10 g/dL to determine that the patient 910 has at least a threshold probability of being diagnosed with severe aplastic anemia. Also, the computing system 902 can determine that the MCV levels of the patient 910 are greater than about 80 fL and that the total hemoglobin levels of the patient 910 are from about 10 g/dL to about 12 g/dL to determine that the patient 910 has at least a threshold probability of being diagnosed with moderate aplastic anemia.

Additionally, the computing system 902 can perform, at operation 948, treatment option identification. Treatment option identification can include determining one or more candidate treatments for a patient 910 with respect to one or more biological conditions. The computing system 902 can determine one or more treatment options for a patient 910 based on a diagnosis of a patient 910 by a healthcare practitioner 928. The computing system 902 can also determine options for the treatment of a biological condition based on a potential diagnosis of an individual with the biological condition, where the potential diagnosis can be included in a patient assessment performed by the computing system 902 at operation 946. For example, the computing system 902 can determine that a patient 910 has at least a threshold probability of having a respective biological condition and, with respect to operation 948, the computing system 902 can then determine a candidate treatment for the respective biological condition. In one or more illustrative examples, the computing system 902 can determine, at operation 948, that a treatment for a patient with iron deficiency anemia can include taking a nutritional supplement that includes one or more forms of iron daily.

The computing system 902 can use one or more additional computational models, at operation 948, to determine one or more candidate treatments for a biological condition. The one or more additional computational models used by the computing system 902, at operation 948, can be different from the one or more models used at operation 946 to perform the one or more patient assessments. The one or more additional models can also be generated by the computing system 902 using one or more machine learning techniques. In various examples, the one or more additional computational models can be generated using additional training data and additional testing data. The additional training data and the additional testing data can be different from testing data and training data used to generate one or more computational models used by the computing system 902 to perform one or more of the patient assessments at operation 946. In various examples, the training data and the testing data used to generate the one or more additional computational models can include data directed to at least one of characteristics of individuals diagnosed with a biological condition, one or more treatments used by the individuals diagnosed with the biological condition, or the effectiveness of the treatments used by the individuals diagnosed with the biological condition.

The computing system 902 can also, at operation 950, monitor one or more patients 910. In one or more implementations, the computing system 902 can, at operation 950, determine whether to send one or more alerts to a patient 910 via the one or more patient computing devices 908 and/or to a healthcare practitioner 928 via one or more practitioner computing devices 926. For example, the computing system 902 can use the sensor data analysis information generated at operation 944 and compare the information to one or more threshold levels. In one or more illustrative examples, the computing system 902 can monitor hemoglobin levels and/or hematocrit levels of a patient 910. Based on the hemoglobin and/or hematocrit levels of the patient 910, the computing system 902 can determine that a patient has at least a threshold probability of being anemic. Subsequently, the computing system 902 can send a practitioner alert 938 to the one or more practitioner computing devices 926 indicating that the patient 910 has at least the threshold probability of being anemic. Additionally, the computing system 902 can send a patient alert 920 to the one or more patient computing devices 908 indicating that the patient 910 has at least the threshold probability of being anemic.

In additional implementations, the computing system 902 can, at operation 950, monitor whether patients 910 are following one or more treatments for a biological condition. For example, the computing system 902 can determine a period of time in which a patient 910 is to perform one or more actions associated with a treatment for the biological condition. The computing system 902 can also determine whether the patient 910 has performed the one or more actions with the period of time. In situations where the computing system 902 determines that a patient 910 did not perform one or more actions associated with a treatment for a biological condition within a period of time, the computing system 902 can generate at least one of a patient alert 920 or a practitioner alert 938 and send the patient alert 920 to the one or more patient computing devices 908 and/or send the practitioner alert 938 to the one or more practitioner computing devices 926. For example, in scenarios where a treatment of anemia for a patient 910 includes daily consuming one or more nutritional supplements, such as an iron supplement, a folic acid supplement, and/or a vitamin B-12 supplement, the computing system 902 can monitor for information received from the one or more patient computing devices 908 and/or from the one or more practitioner computing devices 926 indicating that the patient 910 has consumed the nutritional supplement. In one or more illustrative examples, in the absence of receiving information from the one or more patient computing devices 908 and/or the one or more practitioner computing devices 926 indicating that the nutritional supplement has been consumed within a specified period of time, the computing system 902 can generate and send a patient alert 920 to the one or more patient computing devices 908 to remind the patient 910 to consume the nutritional supplement. The computing system 902 can also generate and send a practitioner alert 938 to the one or more practitioner computing devices 926 indicating that the patient 910 has not consumed the nutritional supplement.

Further, at operation 952, the computing system 902 can perform an analysis of data that is obtained from one or more sources that are external to the computing system 902. The external data can include medical records of one or more individuals. The external data can also include at least one of clinical trials information, journal articles, academic papers, or other medical literature. In various examples, the external data can include laboratory test results of one or more individuals, diagnostic test results of one or more individuals, blood-related information of one or more individuals, vital sign information of one or more individuals, genetic information of one or more individuals, treatment information for one or more biological conditions of one or more individuals, or one or more combinations thereof.

In one or more implementations, the computing system 902 can, at operation 952, use the external data as training data and/or test data to generate one or more computational models. For example, the computing system 902 can analyze data obtained from one or more external sources indicating characteristics of individuals diagnosed with a biological condition to generate one or more computational models that can be used to determine a probability that biological condition is or will be present in an additional individual. In one or more additional examples, the computing system 902 can analyze data obtained from one or more external sources indicating at least one of treatments of individuals diagnosed with a biological condition or effectiveness of the treatments with respect to the biological condition to generate one or more computational models that can be used to identify one or more candidate treatments for one or more additional individuals diagnosed with the biological condition.

In additional implementations, the computing system 902 can, at operation 954, facilitate communications between one or more patients 910 and one or more healthcare practitioners 928. For example, the computing system 902 can obtain communications 922 from the one or more patient computing devices 908 and provide at least a portion of the communications 922 to the one or more practitioner computing devices 926. Additionally, the computing system 902 can obtain communications 940 from the one or more practitioner computing devices 926 and provide at least a portion of the communications 940 to the one or more patient computing devices 908. In various implementations, the computing system 902 can provide one or more user interfaces that can capture text communications, audio communications, video communications, or one or more combinations thereof. The data obtained via the one or more user interfaces can be used to generate at least one of the communications 922 or communications 940. In one or more implementations, the computing system 902 can enable the one or more patient computing devices 908 and the one or more practitioner computing devices 926 to exchange real-time communications, such as video calls, audio calls, and/or text messages.

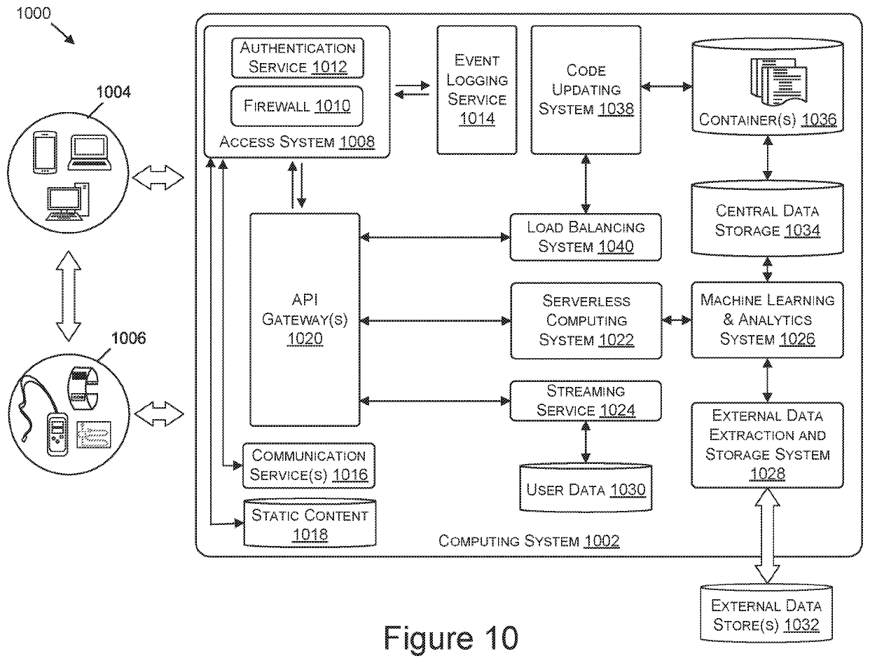

FIG. 10 illustrates an example environment 1000 to obtain and analyze information related to the detection and treatment of one or more biological conditions. The environment 1000 can include a computing system 1002. The computing system 1002 can include all or a portion of the computing system 902 described with respect to FIG. 9. Additionally, at least a portion of the computing system 1002 can be included in the health data system 102 or implemented in relation to the health data system 102. In one or more implementations, the computing system 1002 can be used and/or implemented by a service provider that provides one or more services related to the diagnosis and treatment of a biological condition.

The environment 1000 can also include one or more computing devices 1004 in communication with the computing system 1002. The one or more computing devices 1004 can include one or more mobile computing devices, one or more smart phones, one or more table computing devices, one or more laptop computing devices, one or more desktop computing devices, or one or more combinations thereof. The one or more computing devices 1004 can be operated by one or more healthcare practitioners, one or more patients diagnosed with at least one biological condition, one or more representatives of a service provider implementing the components of the computing system 1002, or one or more combinations thereof.

The environment 1000 can also include one or more data capture devices 1006 that can be in communication with at least one of the computing system 1002 or the one or more computing devices 1004. The one or more data capture devices 1006 can include the wearable device 200 described with respect to FIGS. 2-5 and/or the wearable device 600 described with respect to FIGS. 6-8. In various implementations, the one or more computing devices 1004 can be intermediate devices that communicate information between the one or more data capture devices 1006 and the computing system 1002. For example, a data capture device 1006 can send sensor data and/or other information to a computing device 1004 and the computing device 1004 can forward the information received from the data capture device 1006 to the computing system 1002. In one or more examples, the computing device 1004 can process the data obtained from the data capture device 1006 before sending the processed data to the computing system 1002. To illustrate, a computing device 1004 can format data obtained from a data capture device 1006 and, subsequently, send the formatted data to the computing system 1002. In one or more illustrative examples, the computing device 1004 can format data obtained from the data capture device 1006 according to an extensible markup language (XML) format or a JavaScript object notation (JSON) format.

The one or more data capture devices 1006 can include one or more wearable devices that can be worn on a portion of a body of an individual, one or more monitoring devices that can include a sensor portion and a hand-held portion, one or more lab-on-a-chip devices that can analyze a fluid sample and/or a tissue sample of an individual, or one or more combinations thereof. The one or more data capture devices 1006 can obtain data associated with one or more individuals using one or more sensing devices included in the one or more data capture devices 1006. In one or more illustrative examples, the one or more sensing devices can include at least one of one or more emitters of electromagnetic radiation or one or more detectors of electromagnetic radiation. The one or more sensing devices of the one or more data capture devices 1006 can also include circuitry that produces one or more electrical signals in the presence of one or more biomolecules.

The computing system 1002 can include a number of components including one or more services, one or more data stores, one or more systems, one or more additional components, or one or more combinations thereof. The components included in the computing system 1002 can be implemented on one or more computing devices that can include one or more server computing devices, one or more desktop computing devices, one or more mobile computing devices, or one or more combinations thereof. In various examples, the one or more computing devices used to implement the components of the computing system 1002 can be located in one or more geographic locations. Additionally, in one or more implementations, the one or more computing devices used to implement the components of the computing system 1002 can be administered and/or maintained by one or more entities.

The computing system 1002 can include an access system 1008 that can enable the passage of data into the computing system 1002 and out of the computing system 1002. The access system 1008 can include a firewall 1010 that controls the flow of data into the computing system 1002 and the flow of data out of the computing system 1002 according to a number of network security rules. The firewall 1010 can enable various types of data and/or communications into the computing system 1002 and can also restrict the flow of additional types of data and/or communications out of the computing system 1002. The access system 1008 can also include an authentication service 1012 that can operate in conjunction with the firewall 1010 to control the flow of data into and out of the computing system 1002. For example, the authentication service 1012 can verify credentials of computing devices 1004 sending data to the computing system 1002. In various examples, the authentication service 1012 can limit access to the computing system 1002 to a group of computing devices 1004 that have been granted access to the computing system 1002. For example, the authentication service 1012 can identify one or more authentication tokens corresponding to data sent to the computing system 1002 by a computing device 1004 and verify the one or more authentication tokens before granting access by the computing device 1004 to the computing system 1002. After verifying credentials of the computing device 1004, the authentication service 1012 can register the computing device 1004 with the computing system 1002. In one or more examples, the credentials of the computing device 1004 can be generated by an application executed by the computing device 1004. In one or more implementations, the access system 1008 can implement one or more blockchain technologies to control access to the computing system 1002.

The authentication service 1012 can also authenticate and register one or more data capture devices 1006. In one or more implementations, the authentication service 1012 can receive credentials corresponding to a data capture device 1006 and verify the credentials of the data capture device 1006. In various examples, the authentication service 1012 can directly receive the credentials from the data capture device 1006. In additional examples, the authentication service 1012 can receive the credentials of the data capture device 1006 from an intermediate device, such as a computing device 1004. In one or more implementations, the credentials of the data capture device 1006 can be stored by the data capture device 1006 and/or generated by the data capture device 1006. In additional examples, the credentials of the data capture device 1006 can be generated by an application executed by an intermediate computing device 1004. Further, after being authenticated by the authentication service 1012, a data capture device 1006 can be registered with the computing system 1002 by the authentication service 1012.

In one or more additional implementations, the computing system 1002 can include an event logging service 1014. The event logging service 1014 can log events related to the computing system 1002 receiving data from at least one of the one or more computing devices 1004 or the one or more data capture devices 1006. For example, the event logging service 1014 can generate a log of sensor data produced by a data capture device 1006 and received by the computing system 1002. In various examples, the event logging service 1014 can also determine one or more actions for the computing system 1002 to perform with respect to data received by the computing system 1002. In additional examples, the event logging service 1014 can operate in conjunction with one or more additional components of the computing system 1002 to determine one or more actions for the computing system 1002 to perform with respect to data received by the computing system 1002. In one or more illustrative examples, the event logging service 1014 can determine that sensor data obtained from a data capture device 1006 is to be forwarded to a persistent memory storage device of the computing system 1002. Further, the event logging service 1014 can determine that sensor data obtained from a data capture device 1006 is to be sent to a real-time monitoring service and/or a near real-time monitoring service of the computing system 1002 such that the sensor data can be accessed by one or more individuals, such as a healthcare practitioner and/or a patient, via an application executed by at least one computing device 1004. The event logging service 1014 can log the receipt of large amounts of sensor data by the computing system 1002, such as thousands up to millions of sensor data readings per second.

The authentication service 1012 can also authenticate individual users of the computing system 1002. Users of the computing system 1002 can be authenticated by credentials of the users with the computing system, such as a user identifier, one or more passcodes, biometric information, voice information, or one or more combinations thereof. In one or more examples, the authentication service 1012 can authenticate healthcare practitioners to access the computing system 1002. Healthcare practitioners can access the computing system 1002 to obtain information related to sensor data of patients of the healthcare practitioner, to communicate with patients of the healthcare practitioner, to access analytics tools provided by the computing system 1002, to access medical literature and other information related to one or more biological conditions, or one or more combinations thereof. Additionally, the authentication service 1012 can authenticate patients to access the computing system 1002. Patients can access the computing system 1002 to obtain information related to sensor data of the patients, to communicate with one or more healthcare practitioners, to obtain one or more treatment plans for a biological condition, to access medical information related to one or more biological conditions, or one or more combinations thereof.

The computing system 1002 can include one or more communication services 1016 that can exchange communications between computing devices 1004 of healthcare practitioners and patients of the healthcare practitioners that have been authenticated by the access system 1008. The one or more communication services 1016 can receive data corresponding to communications from one or more senders and route the communication data to one or more recipients. The one or more communication services 1016 can obtain communications data that is captured via one or more user interfaces and/or one or more input devices of the computing devices 1004. The one or more user interfaces can capture text data of communications exchanged between one or more recipients and one or more senders. The one or more input devices can include one or more microphones that can capture audio data related to voice communications. The one or more input devices can also include one or more cameras that can capture video data related to visual communications.

In addition, the computing system 1002 can include static content 1018 that can be provided to one or more computing devices 1004 and/or one or more data capture devices 1006. The static content can include content of one or more webpages. The static content 1018 can also include content that is displayed in conjunction with the execution of one or more mobile device applications that can be in communication with the computing system 1002. In various examples, the static content 1018 provided to a respective computing device 1004 can be based on a location of the computing device 1004. For example, a first computing device 1004 located in a first location, such as a first country, can obtain webpages and/or mobile device application content that is different from content obtained by a second computing device 1004 located in a second location, such as a second country. Additionally, static content 1018 provided to healthcare practitioners can differ from static content 1018 provided to patients of healthcare practitioners. To illustrate, the appearance and functionality provided via a mobile device application for healthcare practitioners can be different from the appearance and functionality provided via a mobile device application of a patient.

In various implementations, the computing system 1002 can include one or more application programming interface (API) gateways 1020. The one or more API gateways 1020 can receive at least one of API calls or API requests from the one or more computing devices 1004 and/or the one or more data capture devices 1006. The one or more API gateways 1020 can invoke one or more additional components of the computing system 1002 based on the API calls and/or the API requests received by the computing system 1002. For example, the one or more API gateways 1020 can invoke the one or more communication services 1016 based on receiving one or more API calls and/or requests related to communications between one or more healthcare practitioners and one or more patients. Additionally, the one or more API gateways 1020 can cause one or more portions of the static content 1018 to be provided to at least one of one or more computing devices 1004 or one or more data capture devices 1006. Further, the one or more API gateways 1020 can invoke additional components of the computing system 1002, such as the serverless computing system 1022 and/or the streaming service 1024 in response to one or more API calls and/or requests received by the computing system 1002. In various implementations, the one or more API gateways 1020 can invoke the serverless computing system 1022 to invoke additional components of the computing system 1002, such as the machine learning and analytics system 1026 and the external data extraction and storage system 1028.

The serverless computing system 1022 can receive one or more requests from the one or more API gateways 1020 and can determine, based on the requests, one or more functions and/or one or more services to perform operations related to the one or more requests. In situations where the serverless computing system 1022 receives requests to access information generated by one or more machine learning models, the serverless computing system 1022 can invoke the machine learning and analytics system 1026. For example, the serverless computing system 1022 can receive a request from the one or more API gateways 1020 to invoke the machine learning and analytics system 1026 to determine a probability that an individual is anemic based on sensor data obtained from a data capture device 1006.

The streaming service 1024 can operate in conjunction with the access system 1008 to store sensor data obtained from at least one of the one or more data capture devices 1006 or the one or more computing devices 1004. The streaming service 1024 can store sensor data in relation to user data 1030. To illustrate, the streaming service 1024 can store sensor data in association with an identifier of a patient and/or an identifier of a healthcare practitioner. In this way, the patient and/or a healthcare practitioner associated with the patient can access sensor data or information related to the sensor data from the computing system 1002. In addition to identifiers of patients and identifiers of healthcare practitioners, the user data 1030 can include medical records of patients, laboratory test results of patients, diagnostic test results of patients, imaging data of patients, genetic data of patients, contact information of patients, contact information of healthcare practitioners, or one or more combinations thereof.

The machine learning and analytics system 1026 can perform one or more operations related to descriptive analytics to describe data trends over a period of time. Additionally, the machine learning and analytics system 1026 can perform one or more operations related to predictive analytics to predict one or more probabilities of one or more events taking place. Further, the machine learning and analytics system 1026 can perform one or more operations related to prescriptive analytics to provide recommendations for one or more candidate actions that patients and/or healthcare practitioners can take with regard to treating a biological condition. In one or more implementations, the machine learning and analytics system 1026 can generate and implement one or more computational models to perform at least one of descriptive analytics, predictive analytics, or prescriptive analytics. The machine learning and analytics system 1026 can also analyze interactions between the computing system 1002 and patients and healthcare practitioners to determine responses by the computing system 1002 to input provided to the computing system 1002 from patients and healthcare practitioners. In one or more additional implementations, the machine learning and analytics system 1026 can analyze geospatial data to perform descriptive analytics, predictive analytics, and/or prescriptive analytics in relation to geographic information as it pertains to one or more biological conditions.

In one or more illustrative examples, the machine learning and analytics system 1026 can analyze sensor data from a number of data capture devices 1006 to determine trends in blood-related data of a number of individuals. For example, the machine learning and analytics system 1026 can determine increases and/or decreases in at least one of hemoglobin levels or hematocrit levels of a number of individuals. The machine learning and analytics system 1026 can also analyze blood-related information of a number of individuals to generate a computational model to determine a probability that one or more individuals are anemic. In addition, the machine learning and analytics system 1026 can analyze blood-related information of a number of individuals and treatment information of a number of individuals to determine one or more candidate treatments for one or more individuals that have at least a threshold probability of being anemic. Further, the machine learning and analytics system 1026 can analyze blood-related information of a number of individuals in conjunction with geographic information of the number of individuals to determine locations that have at least a threshold number of individuals diagnosed with anemia. That is, the machine learning and analytics system 1026 can generate one or more heat maps indicating regions that have at least a threshold number of individuals diagnosed with anemia.

The external data extraction and storage system 1028 can extract and store data obtained from one or more external data stores 1032. The one or more external data stores 1032 can store information related to biological conditions. For example, the one or more external data stores 1032 can store medical literature, clinical trials information, journal articles, academic papers, books, or one or more combinations thereof. The one or more external data stores 1032 can also store medical records information. To illustrate, the one or more external data stores 1032 can store laboratory test results, imaging data, observations of healthcare practitioners, diagnostic test results, treatment protocols, diagnoses of biological conditions, sensor data, or one or more combinations thereof.

At least a portion of the information stored by the one or more external data stores 1032 can be related to individuals for which sensor data from the one or more data capture devices 1006 is obtained by the computing system 1002. In one or more illustrative examples, the one or more external data stores 1032 can store health information obtained by one or more healthcare practitioners that have been visited by individuals for which the computing system 1002 has obtained sensor data from the one or more data capture devices 1006. In additional implementations, at least a portion of the information stored by the one or more external data stores 1032 can be information that is not related to individuals for which the computing system 1002 has received sensor data from the one or more data capture devices 1006. For example, the one or more external data stores 1032 can store health information of a number of individuals that have been diagnosed with a biological condition, such as anemia, but have not provided sensor data to the computing system 1002. In these situations, the health information of the number of individuals can be included in clinical trials information and/or included in research publications.

In one or more implementations, the information stored by the one or more external data stores 1032 can be accessed by the external data extraction and storage system 1028 via one or more websites. In various examples, the external data extraction and storage system 1028 can implement one or more web crawlers to obtain information from the one or more external data stores 1032. In additional implementations, the external data extraction and storage system 1028 can use authentication credentials to access the one or more external data stores 1032. In these situations, the authentication credentials can provide access by the computing system 1002 to information stored by at least a portion of the one or more external data stores 1032.

The data stored by the one or more external data stores 1032 can be structured data, in one or more implementations. Additionally, at least a portion of the data stored by the one or more external data stores 1032 can be unstructured data. In situations where the data stored by the one or more external data stores 1032 is structured data, the external data extraction and storage system 1028 can use one or more queries to obtain information from the one or more external data stores 1032. In scenarios where the data stored by the one or more external data stores 1032 is unstructured data, the external data extraction and storage system 1028 can use one or more natural language processing techniques to retrieve specified information from the one or more external data stores 1032. For example, the external data extraction and storage system 1028 can use one or more natural language processing techniques to parse information obtained from the one or more external data stores 1032 to identify one or more keywords.

In various examples, at least a portion of the information obtained from the one or more external data stores 1032 can be stored by central data storage 1034. The central data storage 1034 can include one or more data storage devices that are coupled to or otherwise accessible to the computing system 1002. In one or more implementations, the one or more data storage devices that comprise the central data storage 1034 can be located in one or more locations. The central data storage 1034 can also store information related to patients that use the one or more data capture devices 1006. For example, the central data storage 1034 can store medical records, laboratory test results, clinical trials information, diagnostic test results, imaging data, genetic information, physical activity information, food and/or liquid consumption information, medication consumption information, nutritional supplement consumption information, vital signs information, sensor data, or one or more combinations thereof, of one or more patients that use a data capture device 1006 that provides sensor information to the computing system 1002. In additional examples, the central data storage 1034 can store system access information and/or account information of patients for which sensor data is being sent from one or more data capture devices 1006 used by the patients to the computing system 1002. The central data storage 1034 can also store information related to healthcare practitioners that access and/or use the computing system 1002. To illustrate, the central data storage 1034 can store system access information and/or account information of healthcare practitioners that use at least a portion of the functionality provided by the computing system 1002. Further, the central data storage 1034 can store enterprise information for one or more service providers that implement at least a portion of the functionality provided by the computing system 1002. In one or more illustrative examples, the central data storage 1034 can store at least a portion of the static content 1018 and/or at least a portion of the user data 1030. The central data storage 1034 can also store information using one or more blockchain technologies such that the information can be stored securely and such that the information can be verifiable.

The central data storage 1034 can also store one or more containers 1036. The one or more containers 1036 can include software code that can be executed by at least one of the computing system 1002 or the one or more computing devices 1004 to perform one or more operations. In one or more implementations, the one or more containers 1036 can include computer-readable instructions that can be executed to perform one or more operations related to one or more services and/or systems implemented by the computing system 1002, such as the access system 1008, the firewall 1010, the authentication service 1012, the event logging service 1014, the one or more communication services 1016, the one or more API gateways 1020, the serverless computing system 1022, the streaming service 1024, the machine learning and analytics system 1026, and/or the external data extraction and storage system 1028. Additionally, the one or more containers 1036 can store computer-readable instructions that can be executed to perform one or more operations related to an application executable by the one or more computing devices 1004. For example, the one or more containers 1036 can store computer-readable instructions that are executable by a computing device 1004 in conjunction with a mobile device application that can send sensor data to the computing system 1002. The one or more containers 1036 can also store computer-readable instructions that are executable by a computing device 1004 in conjunction with a mobile device application that can provide information to patients, such as information related to a probability that an individual has a biological condition and/or information corresponding to one or more treatments for the biological condition. Additionally, the one or more containers 1036 can store computer-readable instructions that are executable by a computing device 1004 in conjunction with an application that can provide information to healthcare practitioners, such as patient monitoring information, patient alerts, and/or patient communications. Further, the one or more containers 1036 can also store computer-readable instructions that are executable by a data capture device 1006.

The computing system 1002 can include a code updating system 1038 that can update the one or more containers 1036. In one or more examples, the code updating system 1038 can include a continuous integration component and a continuous deployment component. In this way, as new software code is developed related to at least one of patches, updates, or new functionality to be implemented by the computing system 1002, by an application executable by the one or more computing devices 1004, or by an application executable by the one or more data capture devices 1006, the new code can be tested and/or deployed in a manner that minimizes the impact of the code changes to other services and/or systems that are implemented by the computing system 1002.

In addition, the computing system 1002 can include a load balancing system 1040 that can control and/or manage the execution of software code included in the one or more containers 1036. For example, the load balancing system 1040 can determine one or more operations to be performed by the computing system 1002 and identify one or more of the one or more containers 1036 that include software code that is executable to perform the one or more operations. In an illustrative example, the load balancing system 1040 can determine that one or more operations are to be performed by the one or more communication services 1016 and identify the one or more containers 1036 that include software code that is executable to perform the one or more operations of the one or more communication services 1016.

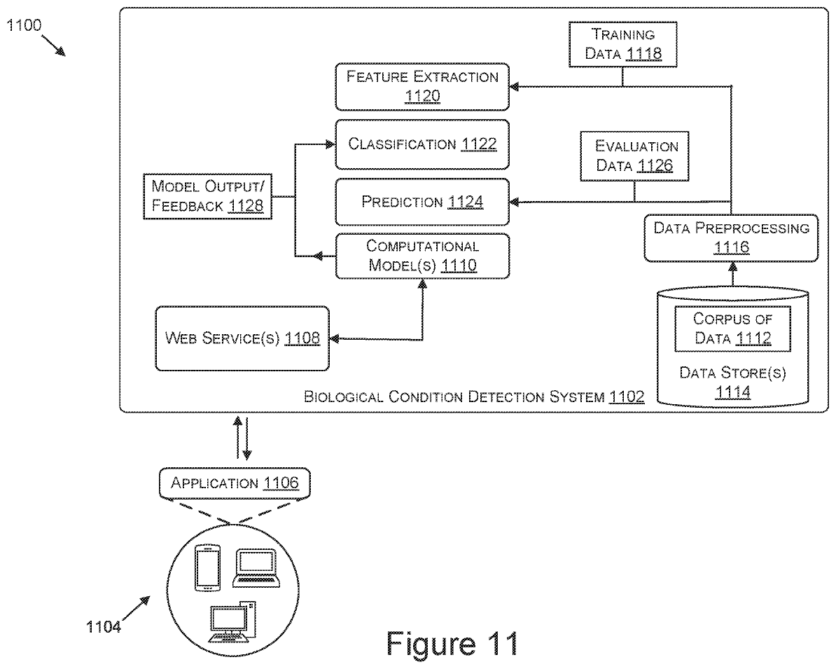

FIG. 11 illustrates an example framework 1100 to generate one or more computational models that can determine a probability that a biological condition is present in an individual. For example, the example framework 1100 can be used to generate one or more computational models that can determine a probability that an individual is anemic, a type of anemia associated with the individual, and/or determine one or more candidate treatments for anemia. The framework 1100 can include biological condition detection system 1102. The biological condition detection system 1102 can be implemented as at least a portion of the health data system 102, a portion of the computing system 902, and/or a portion of the computing system 1002. Additionally, the framework 1100 can include one or more computing devices 1104 and an application 1106 that is executable by the one or more computing devices 1104. In various examples, one or more instances of the application 1106 can be executed by a single computing device 1104. The biological condition detection system 1102 can include one or more web services 1108 that can enable the biological condition detection system 1102 to be in communication with the application 1106. In one or more illustrative examples, the application 1106 can include a mobile device application that includes computer-readable instructions that are executable by the computing device 1104 to perform operations in relation to the detection and treatment of anemia biological condition. In one or more additional examples, the application 1106 can include a browser application that includes computer-executable instructions that are executable to cause the computing device 1104 to access information provided by the biological condition detection system 1102 using one or more webpages.

The application 1106 can cause the computing device 1104 to send sensor data to the biological condition detection system 1102. The sensor data can be obtained by the computing device 1104 from one or more sensors. The one or more sensors can be a part of the computing device 1104. In additional implementations, the one or more sensors can be external with respect to the computing device 1104. For example, the one or more sensors can be included in one or more data capture devices that are in communication with the computing device 1104. To illustrate, the computing device 1104 can obtain sensor data from one or more data capture devices via at least one of a Bluetooth communication protocol or an IEEE 1102.11 communication protocol. After the sensor data has been obtained by the computing device 1104. To illustrate, the computing device 1104 can obtain sensor data from one or more data capture devices via at least one of a Bluetooth communication protocol or an IEEE 1102.11 communication protocol. After the sensor data has been obtained by the computing device 1104, the computing device 1104 can then send the sensor data to the biological condition detection system 1102. In various examples, the sensor data can include data indicating wavelength ranges of electromagnetic radiation emitted by one or more emitter devices, intensity values of electromagnetic radiation emitted by the one or more emitter devices, intensity values of electromagnetic radiation detected by one or more detector devices, attenuation values of one or more wavelength ranges of electromagnetic radiation, or one or more combinations thereof.

The application 1106 can send one or more requests to the biological condition detection system 1102 to obtain information related to one or more predictions of a diagnosis of a biological condition for an individual. In one or more illustrative examples, the biological condition detection system 1102 can receive requests from the application 1106 to obtain information related to at least one of a probability of anemia being present in an individual, one or more determinations of a type of anemia for an individual, or one or more recommendations for one or more candidate treatments for anemia. The biological condition detection system 1102 can use one or more computational models 1110 to generate responses to requests received from the application 1106. For example, the biological condition detection system 1102 can determine a probability that an individual has anemia based on blood-related information that is associated with the individual. The blood-related information of the individual can be derived from sensor data corresponding to the individual. The one or more computational models 1110 can also use blood-related information of an individual to determine a type of anemia that corresponds to the individual. To illustrate, the one or more computational models 1110 can use blood-related information of an individual to determine a probability that an individual has iron deficiency anemia or a probability that the individual has anemia caused by a chronic disease. Further, the one or more computational models 1110 can analyze blood-related information of the individual to determine one or more treatments for anemia of the individual.

The one or more computational models 1110 can also use additional information, such as biological characteristics, to determine a probability that an individual has a biological condition. For example, the one or more computational models 1110 can use additional information to determine a probability of anemia being present in an individual, to determine a type of anemia of the individual, and/or to determine one or more candidate treatments for anemia of the individual. In one or more illustrative examples, the one or more computational models 1110 can use biological characteristics of individuals to determine a probability that an individual has anemia, to determine a type of anemia of the individual, and/or to determine one or more candidate treatments for anemia of the individual. The biological characteristics can include physical features of the individual, such as age, height, weight, gender, pregnancy status, and/or menstruation status. The biological characteristics can also include genetic information of the individual. Further, the biological characteristics can include levels of analytes of the individual. In addition to the blood-related information of the individual, the levels of analytes of the individual can include at least one of levels of one or more proteins, levels of one or more vitamins, or levels of one or more nutrients. In one or more implementations, the biological characteristics of the individual can also include cardiovascular information of the individual. In various examples, the biological characteristics of the individual can be determined by the biological condition detection system 1102 based on medical records of the individual, laboratory test results of the individual, diagnostic test results of the individual, observations made by one or more healthcare practitioners about the individual, or one or more combinations thereof.

The characteristics of individuals used by the one or more computational models 1110 to determine a probability that a biological condition is present in an individual and/or to determine one or more candidate treatments for the biological condition can be based on one or more variables and/or one or more nodes included in the one or more computational models 1110. The one or more variables and/or one or more nodes included in the one or more computational models 1110 in addition to one or more weights associated with the one or more variables and/or one or more nodes can be determined through an analysis of a corpus of data 1112 that can be stored by one or more data stores 1114 coupled to or otherwise in electronic communication with the biological condition detection system 1102. The corpus of data 1112 can include information obtained from the one or more computing devices 1104 related to the health of individuals, such as blood-related information of the individuals and/or biological characteristics of the individuals. The corpus of data 1112 can also include sensor data obtained from at least one of the one or more computing devices 1104 or one or more data capture devices in communication with the biological condition detection system 1102. Additionally, the corpus of data 1112 can include information obtained from data sources that are external to the biological condition detection system 1102, such as one or more publicly accessible data sources and/or one or more privately accessible data sources. In various examples, the corpus of data 1112 can include information of individuals that have previously been diagnosed with anemia. Further, the corpus of data 1112 can include information related to the treatment of individuals previously diagnosed with anemia. In one or more illustrative examples, at least a portion of the corpus of data 1112 can be obtained by the external data extraction and storage system 1028 of FIG. 10.

The biological condition detection system 1102 can perform data preprocessing at operation 1116. The data preprocessing can include one or more operations to modify at least a portion of the corpus of data 1112 such that information included in the corpus of data 1112 can be analyzed to generate the one or more computational models 1110. The data preprocessing can include formatting at least a portion of the corpus of data 1112 according to one or more formatting schemes. In various examples, the data preprocessing can standardize the format of the information included in at least a portion of the corpus of data 1112. Additionally, the data preprocessing can include at least one of aggregating one or more portions of the corpus of data 1112 or separating one or more portions of the corpus of data 1112.

After performing the data preprocessing at operation 1116, a portion of the corpus of data 1112 can be used as training data 1118 to generate the one or more computational models 1110. In one or more implementations, the biological condition detection system 1102 can analyze the training data 1118 by performing feature extraction at operation 1120 and classification at operation 1122. Feature extraction at operation 1120 can include identifying one or more variables and/or one or more sets of variables that can be used to make one or more predictions based on a set of input data. The feature extraction can determine relationships between one or more variables included in the training data 1118 and determine one or more measures of correlations between variables and/or groups of variables included in the training data 1118. The classification that takes place at operation 1122 can include classifying one or more pieces of information included in the training data 1118 according to one or more categories.

The feature extraction and classification operations can be used to determine one or more initial computational models that can make one or more predictions at operation 1124. The predictions that take place at operation 1124 can implement the initial computational models produced by the feature extraction at operation 1120 and the classification at operation 1122 with respect to evaluation data 1126. The evaluation data 1126 can be used to evaluate the performance of the initial computational models produced by the feature extraction at operation 1120 and the classification at operation 1122. The evaluation data 1126 can include a portion of the corpus of data 1112 that is different from the training data 1118. In this way, the evaluation data 1126 that is used to evaluate the performance of the initial computational models is different from the training data 1118 that is used to generate the one or more initial computational models. Based on the performance of the initial computational models implemented with the evaluation data 1126, the feature extraction and/or classification can be performed one or more additional times until the one or more computational models 1110 are produced. The one or more computational models 1110 can be produced after one or more iterations of feature extraction, classification, and prediction using the evaluation data 1126. In one or more implementations, the one or more computational models 1110 can be produced after the feature extraction, classification, and prediction produce computational models that satisfy one or more performance criteria, such as one or more convergence criteria or one or more accuracy criteria.

After the one or more computational models 1110 have been generated, the one or more computational models 1110 can be further refined as additional data is obtained by the biological condition detection system 1102. For example, as additional sensor data, additional blood-related information, and/or additional biological characteristics of individuals are obtained by the biological condition detection system 1102, the accuracy of the one or more computational models 1110 can be further evaluated. In various examples, as the new data is obtained by the biological condition detection system 1102, model output/feedback 1128 can be produced that is then fed back to the classification operation at 1122. In this way, the new data can be used to improve the predictions made at operation 1124 by modifying one or more variables, one or more nodes, and/or one or more weights of the one or more computational models 1110.

The biological condition detection system 1102 can use one or more machine learning techniques to generate the one or more computational models 1110. For example, the one or more computational models 1110 can include one or more neural networks. To illustrate, the one or more computational models 1110 can include at least one of one or more convolutional neural networks, one or more recurrent neural networks, or one or more multilayer perceptron neural networks. Additionally, at least a portion of the one or more computational models 1110 can be generated using a computational platform that analyzes sensor data using one or more machine learning techniques within a microcontroller. In these situations, the data obtained by sensors, such as sensors of a wearable device, can be analyzed on the device itself using one or more machine learning techniques without sending the data to a remotely located (e.g., cloud-based) computational platform. In further implementations, at least a portion of the one or more computational models 1110 can be generated using a computational platform that is remotely located from a device that is collecting data from one or more sensors. In various examples, the one or more computational models 1110 can be generated using a combination of one or more machine learning techniques implemented by a computational platform that operates on a sensor-containing device and a one or more machine learning techniques implemented by a computational platform that operates remotely with respect to the sensor-containing device.

In one or more illustrative examples, the one or more computational models 1110 can determine a probability that an individual may be diagnosed with anemia and the corpus of data 1112 can include information corresponding to individuals that have previously been diagnosed with anemia. For example, the corpus of data 1112 can include blood-related information and/or additional biological characteristics of individuals that have been previously diagnosed with anemia. The biological condition detection system 1102 can analyze a portion of the blood-related information and/or the additional biological characteristics of individuals previously diagnosed with anemia included in the corpus of data 1112 as the training data 1118 using the feature extraction operation 1120, the classification operation 1122, and the prediction operation 1124 to generate the one or more computational models 1110. In this way, the biological condition detection system 1102 can determine one or more portions of blood-related information and/or one or more biological characteristics that can be predictors of one or more types of anemia. Subsequently, as the biological condition detection system 1102 obtains additional information about additional individuals, the biological condition detection system 1102 can extract the relevant portions of the additional information that correspond to components of the one or more computational models 1110 and the implement the one or more computational models 1110 with the relevant portions of the additional information to determine a probability that the additional individuals are anemic and/or to determine a probability of one or more individuals being diagnosed with a respective type of anemia.

In one or more additional illustrative examples, the one or more computational models 1110 can determine one or more recommendations for one or more candidate treatments with respect to a biological condition. To illustrate, the corpus of data 1112 can include anemia treatment information indicating at least one of one or more treatments of one or more types of anemia, one or more measures of effectiveness of at least a portion of the one or more treatments, or one or more characteristics of individuals that have received the one or more treatments. The biological condition detection system 1102 can analyze a portion of the anemia treatment information included in the corpus of data 1112 as the training data 1118 using the feature extraction operation 1120, the classification operation 1122, and the prediction operation 1124 to generate the one or more computational models 1110. In this way, the biological condition detection system 1102 can determine one or more characteristics of individuals that can be used to identify one or more candidate treatments for anemia. For example, the biological condition detection system 1102 can determine that individuals with first hemoglobin levels can be treated using one or more first candidate treatments and individuals with second hemoglobin levels can be treated using one or more second candidate treatments. Subsequently, as the biological condition detection system 1102 obtains anemia treatment information about additional individuals, such as additional hemoglobin levels, the biological condition detection system 1102 can extract the relevant portions of the additional information and implement the one or more computational models 1110 with the relevant portions of the additional information to determine one or more candidate treatments for the additional individuals.

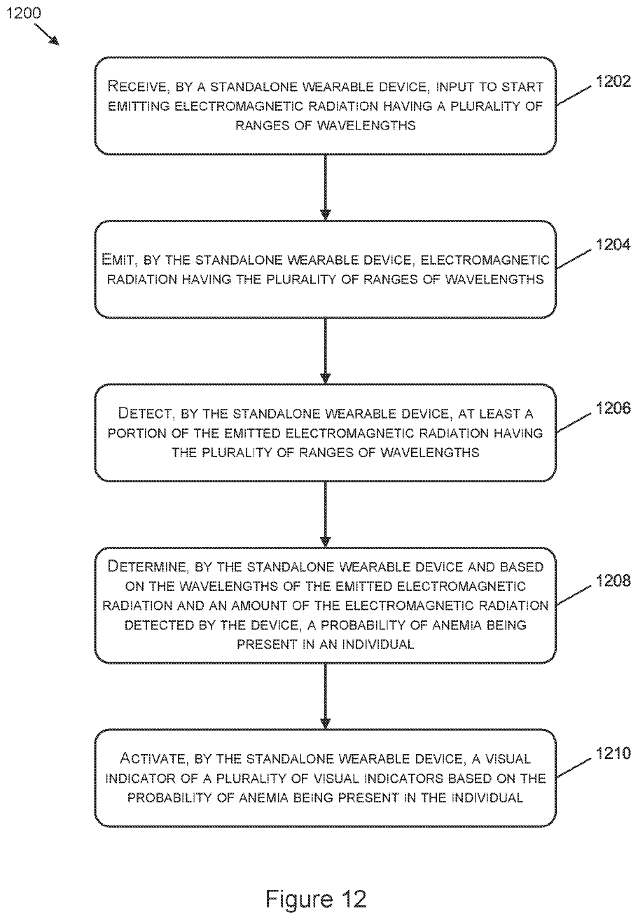

FIGS. 12-14 illustrate example methods for detecting the presence or absence of one or more biological conditions in individuals. The example processes are illustrated as collections of blocks in logical flow graphs, which represent sequences of operations that can be implemented in hardware, software, or a combination thereof. The blocks are referenced by numbers. In the context of software, the blocks represent computer-executable instructions stored on one or more computer-readable media that, when executed by one or more processing units (such as hardware microprocessors), perform the recited operations. Generally, computer-executable instructions include routines, programs, objects, components, data structures, and the like that perform particular functions or implement particular data types. The order in which the operations are described is not intended to be construed as a limitation, and any number of the described blocks can be combined in any order and/or in parallel to implement the process.

FIG. 12 is a flow diagram of an example process 1200 for a standalone wearable device to use sensor data to provide an indication of a level of anemia for an individual. The process 1200 can include, at operation 1202, receiving, by a standalone wearable device, input to start emitting electromagnetic radiation having a plurality of ranges of wavelengths. Each wavelength range of the plurality of wavelength ranges can comprise a different subset of wavelengths from about 300 nm to about 1500 nm. The wearable device can be referred to as a standalone wearable device herein due to the data collection and at least a portion of the data analysis to determine a level of anemia of an individual being performed by the wearable device itself. Although, in various additional implementations, at least a portion of the analysis of data collected by the wearable device can be performed by additional devices, such as a cloud-based computing system, a mobile device of an individual, a health practitioner computing device, and the like. Additionally, the device can be referred to herein as a wearable device based on the device having characteristics such that the device can be worn on a portion of a body of an individual. In one or more implementations, the standalone wearable device can be worn on a finger of an individual.

The input received to start the emission of electromagnetic radiation by the standalone wearable device can be provided via an input device of the standalone wearable device. The input device can include a button of the standalone wearable device. The input device can also include one or more sensors of the standalone wearable device. The one or more sensors can include at least one touch sensor. Additionally, the one or more sensors can include at least one proximity sensor. In various implementations, placing the standalone wearable device on a finger of an individual can generate the input that causes the standalone wearable device to start emitting electromagnetic radiation. In further implementations, the input to activate the standalone wearable device to start emitting electromagnetic radiation can be received from a computing device that is not physically coupled to the standalone wearable device. To illustrate, the standalone wearable device can receive input to start emitting electromagnetic radiation from a mobile computing device via a wireless communication interface of the standalone wearable device. The mobile computing device can be operated by an individual wearing the standalone wearable device or by a healthcare practitioner. In addition, the mobile device can be executing a mobile device application that collects, analyzes, and displays information related to anemia and information related to the blood of individuals based on data obtained from the standalone wearable device. Also, the standalone wearable device can receive input to start emitting electromagnetic radiation from a monitoring device in communication with the standalone wearable via a wireless communication interface of the standalone wearable device or via a wired communication interface of the standalone wearable device.

At operation 1204, the process 1200 can include emitting, by the standalone wearable device, electromagnetic radiation having the plurality of wavelength ranges. Each wavelength range can include at least one wavelengths of electromagnetic radiation. The plurality of wavelength ranges can include wavelengths of electromagnetic radiation ranges within about 300 nm to about 1500 nm. In one or more illustrative examples, a first wavelength range can be from about 540 nm to about 690 nm, a second wavelength range can be from about 710 to about 770 nm, a third wavelength range can be from about 780 nm to about 840 nm; a fourth wavelength can be from about 810 nm to about 900 nm, and a fifth wavelength range can be from about 910 nm to about 990 nm.

In one or more implementations, the electromagnetic radiation can be emitted by one or more LEDs of the standalone wearable device. The plurality of wavelength ranges of electromagnetic radiation can be emitted according to one or more protocols that indicate a sequence and/or timing of the emission of individual wavelength ranges of the plurality of wavelength ranges. For example, an electromagnetic radiation emission protocol can indicate an amount of time to emit one or more wavelength ranges. An electromagnetic emission protocol can also indicate an amount of time to pause between the emission of different wavelength ranges. Further, an electromagnetic emission protocol can indicate one or more combinations of wavelength ranges to be emitted sequentially or to be emitted during one or more overlapping time periods. In one or more illustrative examples, individual wavelength ranges of electromagnetic radiation can be emitted for substantially a same period of time. In one or more additional illustrative examples, individual wavelength ranges of electromagnetic radiation can be emitted for a time period that is different from a time period that other wavelength ranges of electromagnetic radiation are emitted. In various implementations, the electromagnetic radiation can be emitted in pulses for a period of time from about 50 microseconds to about 500 microseconds. At least one example electromagnetic radiation emission protocol can indicate that a first wavelength range of electromagnetic radiation is emitted for a first period of time, followed by a pause, a second wavelength range of electromagnetic radiation is emitted for a second period of time after the pause completes, and a third wavelength range of electromagnetic radiation is emitted for a third period of time after another pause that takes place after the electromagnetic radiation having the second wavelength range is completed. The protocol can be repeated one or more times. Additionally, the electromagnetic radiation can be emitted for a minimum amount of time. A maximum threshold of time for the emission of electromagnetic radiation can also be implemented.

The process 1200 can also include, at operation 1206, detecting, by the standalone wearable device, at least a portion of the emitted electromagnetic radiation having the plurality of ranges of wavelengths. In various examples, the electromagnetic radiation emitted by the standalone wearable device can be absorbed by one or more biomolecules in the blood of individuals. The amount of electromagnetic radiation absorbed by the one or more biomolecules can be based on an amount of the one or more biomolecules present in the blood of individuals. For example, as the amount of a biomolecule in the blood of an individual increases, an increased amount of electromagnetic radiation of a wavelength range can be absorbed. As the amount of electromagnetic radiation emitted by the standalone wearable device is absorbed, a decreasing amount of the electromagnetic radiation is detected by the standalone wearable device.

In one or more illustrative examples, emitting and detecting the plurality of wavelength ranges of electromagnetic radiation can be used to determine an amount of oxygenated hemoglobin in the blood of individuals and/or an amount of deoxygenated hemoglobin in the blood of individuals, and or the total amount of hemoglobin in the blood of individuals. Additionally, emitting and detecting with respect to the plurality of wavelength ranges of electromagnetic radiation can be used to determine an amount of methemoglobin in the blood of individuals. Further, emitting and detecting the plurality of wavelength ranges of electromagnetic radiation can be used to determine an amount of carboxyhemoglobin in the blood of individuals. The plurality of wavelength ranges of electromagnetic radiation can also be used to determine additional information about the individuals, such as heart rate information and peripheral capillary oxygen saturation (SpO₂) information.

The electromagnetic radiation can be detected by one or more photodiodes. In various implementations, the standalone wearable device can include one or more photodiodes that detect wavelengths of electromagnetic radiation that span all of the wavelength ranges emitted by the standalone wearable device. In additional implementations, one or more first photodiodes of the standalone wearable device can detect a first portion of the ranges of wavelengths emitted by the standalone wearable device and one or more second photodiodes of the standalone wearable device can detect a second portion of the ranges of wavelengths emitted by the standalone wearable device. In one or more implementations, the standalone wearable device can include one or more respective photodiodes that detect electromagnetic radiation for each wavelength range emitted by the standalone wearable device.

At operation 1208, the process 1200 can include the standalone wearable device determining a level of anemia for an individual. The level of anemia can indicate one or more probabilities of anemia being present in the individual. The level of anemia can be based on the wavelengths of electromagnetic radiation emitted by the standalone wearable device and on an amount of the electromagnetic radiation detected by the standalone wearable device. For example, a range of wavelengths of electromagnetic radiation can be absorbed by a biomolecule and the biomolecule can be indicative of a level of anemia of an individual. To illustrate, a level of anemia can be determined based at least partly on an amount of oxygenated hemoglobin included in the blood of an individual and oxygenated hemoglobin can absorb electromagnetic radiation having a respective range of wavelengths. The standalone wearable device can emit electromagnetic radiation having the respective range of wavelengths over a period of time and an amount of the electromagnetic radiation having the respective range of wavelengths can be detected over the period of time. In various implementations, the electromagnetic radiation having the respective range of wavelengths emitted over the period of time can have a first intensity and the electromagnetic radiation having the respective range of wavelengths detected over the period of time can have a second intensity that is less than the first intensity. The second intensity can be less than the first intensity due to the absorption of at least a portion of the electromagnetic radiation having the respective range of wavelengths absorbed by the oxygenated hemoglobin. In one or more implementations, the difference between the first intensity and the second intensity can be used to determine an amount of attenuation of the electromagnetic radiation having the respective range of wavelengths. The amount of attenuation can be used to determine an amount of oxygenated hemoglobin present in the blood of the individual and the amount of oxygenated hemoglobin can then be used, at least in part, to determine the level of anemia of the individual.

Further, the process 1200 can include, at operation 1210, activating, by the standalone wearable device, a visual indicator of a plurality of visual indicators based on the probability of anemia being present in the individual. For example, each visual indicator of the plurality of visual indicators can correspond to different ranges of probabilities of anemia being present in the individual. To illustrate, a first visual indicator can correspond to a first range of probabilities of anemia being present in the individual, a second visual indicator can correspond to a second range of probabilities of anemia being present in the individual, and a third visual indicator can correspond to a third range of probabilities of anemia being present in the individual. In addition, individual indicators of the plurality of indicators can correspond to a respective color. In one or more illustrative examples, the first visual indicator can include a green light emitting diode (LED), the second visual indicator can include a yellow LED, and the third visual indicator can include a red LED. In further examples, the standalone wearable device can include a display device and the information shown on the display device can include at least one of the visual indicators based on the level of anemia of the individual.

In various implementations, the first level of anemia can correspond to an individual having less than a minimum probability of an individual being diagnosed with anemia or less than a minimum probability of an individual requiring treatment for anemia. In addition, the second level of anemia can correspond to an individual having an intermediate probability of an individual being diagnosed with anemia and/or having an intermediate probability of an individual requiring treatment for anemia. The third level of anemia can correspond to a relatively high probability of an individual being diagnosed with anemia and/or having a relatively high probability of an individual requiring treatment for anemia. Based on the probability of the individual being diagnosed with anemia, a visual indicator that corresponds to the probability can be identified and activated. In one or more implementations, activating the visual indicator can include supplying an amount of power to the visual indicator or supplying an amount of power to a display device that includes the visual indicator.

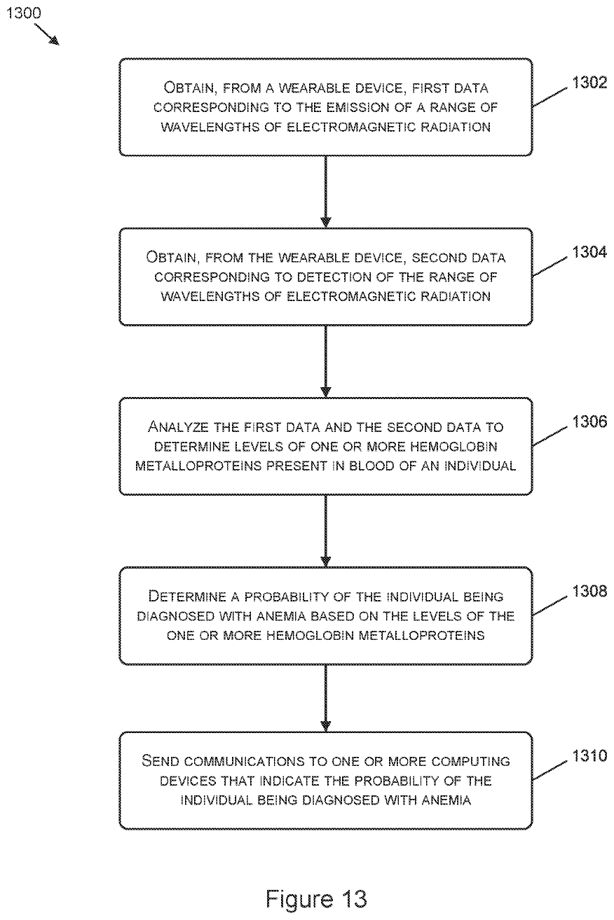

FIG. 13 is a flow diagram of an example process 1300 to analyze electromagnetic emission and detection information to determine a probability of anemia being present in an individual. The example process 1300 can be performed by at least one of a computing system of a computational platform, a computing device of a health care practitioner, or a computing device of an individual that may be diagnosed with anemia. At operation 1302, the process 1300 can include obtaining, from a wearable device, first data corresponding to the emission of a range of wavelengths of electromagnetic radiation. The range of wavelengths of electromagnetic radiation can correspond to electromagnetic radiation that can be absorbed by one or more substances found in the blood of individuals. The one or more substances can include one or more hemoglobin molecules. The one or more hemoglobin molecules can include oxygenated hemoglobin, deoxygenated hemoglobin, methemoglobin, and/or carboxyhemoglobin.

The wearable device can be placed on a finger, a toe, or an ear of an individual. The wearable device can be a standalone wearable device that is not physically coupled to another device. In additional implementations, the wearable device can include a sensor portion that is physically coupled, such as via a wire or connector, to a monitoring device. The wearable device can include a number of emitters to emit electromagnetic radiation at a number of ranges of wavelengths and one or more detectors to detect electromagnetic radiation emitted by the number of emitters.

At operation 1304, the process 1300 can include obtaining, from the wearable device, second data corresponding to detection of the range of wavelengths of electromagnetic radiation. The second data can also include one or more intensities of electromagnetic radiation detected by the wearable device. In one or more examples, the intensity of electromagnetic radiation detected by the wearable device can be less than the electromagnetic radiation emitted by the wearable device. The differences between the amount of electromagnetic radiation emitted by the wearable device and the electromagnetic radiation detected by the wearable device can be based on an amount of the electromagnetic radiation absorbed by one or more biomolecules included in the blood of individuals. The differences between the amount of electromagnetic radiation emitted by the wearable device and the electromagnetic radiation detected by the wearable device can be based on an amount of scattering of the electromagnetic radiation.

In addition, at operation 1306, the process 1300 can include analyzing the first data and the second data to determine levels of one or more hemoglobin metalloproteins present in blood of an individual. Analyzing the first data and the second data can include correlating the electromagnetic radiation emitted by the wearable device with the electromagnetic radiation detected by the wearable device. For example, the first data can include emitted electromagnetic radiation of a range of wavelengths and the second data can include detected electromagnetic radiation of a same or similar range of wavelengths. To illustrate, the first data can include emitted electromagnetic radiation having wavelengths from about 600 nm to about 770 nm and the detected electromagnetic radiation can have wavelengths from about 700 nm to about 770 nm. In various examples, the emitted electromagnetic radiation and the detected electromagnetic radiation can be correlated to one another based on timing information associated with the first data and the second data. The timing information can indicate one or more indicators of time that electromagnetic radiation was emitted and one or more additional indicators of time that electromagnetic radiation was detected. The timing information can also indicate a protocol implemented by the wearable device that indicates one or more patterns of emission and detection of electromagnetic radiation by the wearable device. In this way, the first data and the second data can be correlated and the amount of attenuation of the electromagnetic radiation having a specified wavelength of a specified range of wavelengths can be determined.

Determining levels of one or more hemoglobin metalloproteins can be based on the amount of attenuation of various ranges of wavelengths of electromagnetic radiation. In various examples, as an amount of attenuation of electromagnetic radiation of a range of wavelengths increases, the amount of one or more hemoglobin metalloproteins present in the blood of an individual can also increase. For example, as the amount of attenuation of electromagnetic radiation having wavelengths from about 600 nm to about 770 nm increases, the amount of deoxygenated hemoglobin present in the blood of the individual can also be increased. Additionally, as the amount of attenuation of electromagnetic radiation having wavelengths from about 820 nm to about 990 nm increases, the amount of oxygenated hemoglobin present in the blood of the individual can also be increased. In one or more implementations, the levels of the one or more hemoglobin metalloproteins present in the blood of the individual can be used to determine hematocrit levels of the individual.

Further, at operation 1308, the process 1300 can include determining a probability of the individual being diagnosed with anemia based on the levels of the one or more hemoglobin metalloproteins. The levels of hemoglobin metalloproteins present in the blood of individuals can correspond to probabilities of the individuals being diagnosed with anemia. In one or more implementations, the levels of hemoglobin metalloproteins present in the blood of individuals can be directly correlated with probabilities of individuals being diagnosed with anemia. In one or more additional implementations, the levels of hemoglobin metalloproteins present in the blood of individuals can be indirectly correlated with probabilities of individuals being diagnosed with anemia. In various examples, the levels of hemoglobin metalloproteins present in the blood of individuals can be correlated with probabilities of individuals being diagnosed with anemia based on one or more analyses of one or more corpuses of data. The one or more corpuses of data can indicate hemoglobin metalloprotein levels of individuals that have previously been diagnosed with anemia. Additionally, the one or more corpuses of data can indicate information that can be derived from hemoglobin metalloprotein levels in relation to individuals that have previously been diagnosed with anemia. To illustrate, the one or more corpuses of data can indicate at least one of hematocrit levels, mean corpuscular volume levels, mean corpuscular hemoglobin concentration levels, or red cell distribution levels of individuals previously diagnosed with anemia. In one or more implementations, the one or more corpuses of data can be analyzed to generate one or more computational models that can determine probabilities of individuals being diagnosed with anemia.

At operation 1310, the process 1300 can include sending communications to one or more computing devices where the communications indicate the probability of the individual being diagnosed with anemia. The communications can include emails, messages, calls, or one or more combinations thereof. In additional examples, the communications can include one or more user interfaces that indicate the probability of the individual being diagnosed with anemia. In one or more illustrative examples, probabilities of individuals being diagnosed with anemia can be grouped together. For example, a first number of probabilities of individuals being diagnosed with anemia can be associated with a first category of individuals, a second number of probabilities of individuals being diagnosed with anemia can be associated with a second category of individuals, and a third number of probabilities of individuals being diagnosed with anemia can be associated with a third category of individuals. In one or more illustrative examples, the first category can correspond to a relatively low risk of being diagnosed with anemia, the second category can correspond to an intermediate risk of being diagnosed with anemia, and the third category can correspond to a relatively high risk of being diagnosed with anemia. In various examples, the communications send to the one or more computing devices can include an indicator of a category that corresponds to the probability of the individual being diagnosed with anemia. The indicator of the category can include a visual indicator that includes one or more colors. In additional implementations, the probability of the individual being diagnosed with anemia can include a numerical value that is shown with respect to a scale of probabilities.

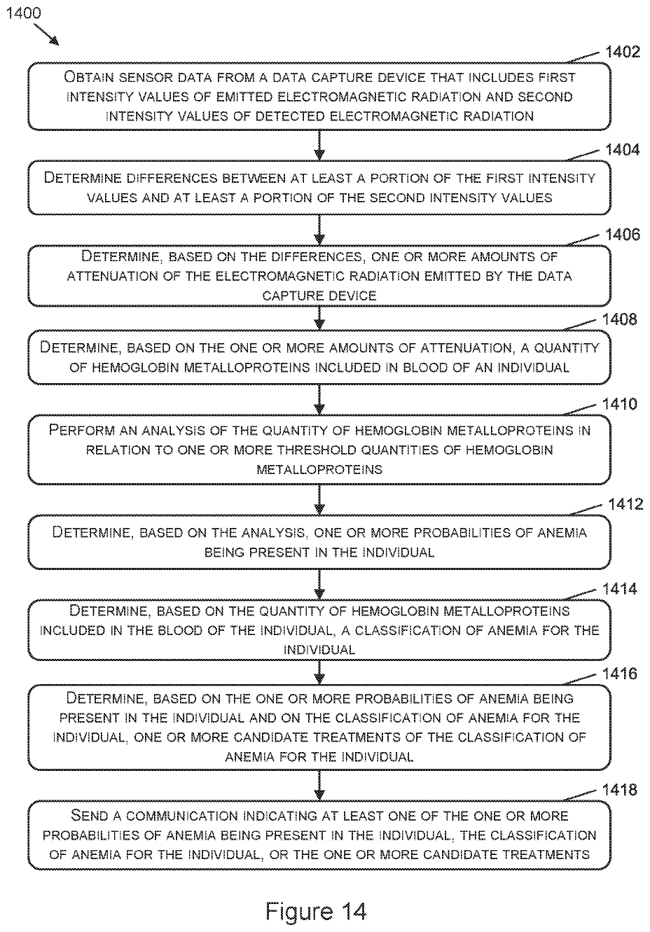

FIG. 14 is a flow diagram of an example process 1400 for a system to analyze at least sensor data to determine a probability of anemia being present in an individual and to determine one or more candidate treatments for the individual with respect to anemia.

The process 1400 can include, at operation 1402, obtaining sensor data from a data capture device. The sensor data can include first intensity values of emitted electromagnetic radiation and second intensity values of detected electromagnetic radiation. In one or more examples, the sensor data can be obtained from a plurality of data capture devices. In additional implementations, at least a portion of the sensor data can be obtained from a computing device that executes an application to collect the sensor data from one or more data capture devices and to send the sensor data to the computing system.

In one or more implementations, authentication data can be obtained from at least one of a computing device or a data capture device. The authentication data can correspond to at least one of an individual or a device that is granted access to exchange information with the computing system. In various examples, at least one of the computing device or a data capture device can be registered with the computing system. Further, the sensor data can be stored in one or more data stores in electronic communication with the computing system. In one or more illustrative examples, the sensor data can be stored in the one or more data stores in association with one or more identifiers of the at least one of the individual, the computing device, or the data capture device.

Additionally, at operation 1404, the process 1400 can include determining differences between at least a portion of the first intensity values and at least a portion of the second intensity values. For example, the computing system can determine differences between the intensity values of one or more first wavelengths of electromagnetic radiation emitted by emitters of the data capture device and the intensity values of one or more second wavelengths of electromagnetic radiation detected by detectors of the data capture device. The process 1400 can then include, at operation 1406, determining, based on the differences between the emitted electromagnetic radiation and the detected electromagnetic radiation, one or more amounts of attenuation of the electromagnetic radiation emitted by the data capture device.

Further, at operation 1408, a quantity of hemoglobin metalloproteins included in blood of an individual can be determined based on the one or more amounts of attenuation. To illustrate, based on the sensor data, first differences between first intensities of first emitted electromagnetic radiation and second intensities of second detected electromagnetic radiation can be determined. In various examples, the first emitted electromagnetic radiation and the second detected electromagnetic radiation can have a first range of wavelengths. In one or more implementations, based on the first differences, a first amount of attenuation of the first emitted electromagnetic radiation can be determined and the first amount of attenuation can be used to determine an amount of oxygenated hemoglobin included in blood of the individual. In additional implementations, based on the sensor data, second differences between third intensities of third emitted electromagnetic radiation and fourth intensities of fourth detected electromagnetic radiation can be determined, where the third emitted electromagnetic radiation and the fourth electromagnetic radiation have a second range of wavelengths. The second differences can be used to determine, based on the second differences, a second amount of attenuation of the third emitted electromagnetic radiation. In one or more implementations, the second amount of attenuation can be used to determine an amount of deoxygenated hemoglobin included in blood of the individual. In one or more illustrative examples, the first range of wavelengths is from about 600 nm to about 770 nm and the second range of wavelengths is from about 820 nm to about 990 nm.

The process 1400 can also include, at operation 1410, performing an analysis of the quantity of hemoglobin metalloproteins in relation to one or more threshold quantities of hemoglobin metalloproteins. The individual threshold quantities of hemoglobin metalloproteins can correspond to a range of probabilities of anemia being present in the individual. In one or more implementations, the process 1400 can include, at operation 1412, determining, based on the analysis, one or more probabilities of anemia being present in the individual. The one or more probabilities of anemia being present in the individual can be based on the measure of the total amount of hemoglobin included in the blood of the individual. A measure of the total amount of hemoglobin being present in the individual can be determined based on the amount of oxygenated hemoglobin in the blood of the individual and the amount of deoxygenated hemoglobin in the blood of the individual. An amount of oxygenated hemoglobin in the blood of the individual or the amount of deoxygenated hemoglobin in the blood of the individual can also be used to determine a level of hematocrit related to the individual. In these scenarios, the one or more probabilities of anemia being present in the individual can be based on the level of hematocrit.

In various examples, the one or more probabilities of anemia being present in the individual can be determined by a computational model. The computational model can be generated by performing an analysis of at least a portion of a corpus of data that indicates a plurality of biological characteristics of individuals that have been diagnosed with anemia. Based on the analysis, a plurality of measures of statistical correlations between groups of biological characteristics included in the plurality of biological characteristics can be determined. The plurality of measures of statistical correlations can be used to determine at least one of one or more variables, one or more nodes, or one or more weights of the computational model. Additionally, based on the sensor data, a feature of the individual can be determined that corresponds to at least one of a variable of the one or more variables or a node of the one or more nodes of the computational model. The feature can include age, height, weight, gender, pregnancy status, menstruation status, a genetic feature, or levels of analytes. In one or more examples, a measure of the feature of the individual can be determined and the computational model can be implemented using the measure of the feature to determine the one or more probabilities of anemia being present in the individual. The computational model can also use additional information to determine one or more probabilities of anemia being present in the individual. In one or more illustrative examples, a measure of oxygenated hemoglobin levels of the individual can be determined based on the amount of attenuation of the range of wavelengths of electromagnetic radiation emitted by the data capture device and the computational model can be implemented using the measure of oxygenated hemoglobin levels of the individual to determine the one or more probabilities of anemia being present in the individual.

At operation 1414, the process 1400 can include determining, based on the quantity of hemoglobin metalloproteins included in the blood of the individual, a classification of anemia for the individual. In one or more illustrative examples, the classification of anemia for the individual can include aplastic anemia, iron deficiency anemia, sickle cell anemia, Thalassemia, vitamin deficiency anemia, anemia caused by chronic disease, and hemolytic anemia. In addition, at operation 1416, the process 1400 can include determining, based on the one or more probabilities of anemia being present in the individual and on the classification of anemia for the individual, one or more candidate treatments of the classification of anemia for the individual. In one or more examples, the one or more candidate treatments can include at least one of a nutritional supplement, a dietary regimen, a pharmaceutical, or a procedure administered by a healthcare practitioner. In one or more additional implementations, the one or more candidate treatments can be determined by generating an additional computational model. The additional computational model can be generated by analyzing an additional corpus of data that includes a plurality of treatments of anemia. In these situations, the computing system can perform an additional analysis of at least a portion of the additional corpus of data to determine a plurality of measures of additional correlations between one or more biological characteristics of the plurality of biological characteristics and one or more treatments of the plurality of treatments. The computing system can also determine, based on the plurality of measures of additional correlations, at least one of one or more additional variables, one or more additional nodes, or one or more additional weights of the additional computational model that can be used to determine at least one recommendation for a candidate treatment of individuals in which anemia is present.

Further, at operation 1418, the process 1400 can include sending a communication to a computing device of the individual. The communication can indicate diagnostic health information, such as the one or more probabilities of anemia being present in the individual. The communication can also indicate the classification of anemia for the individual and/or the one or more recommendations for candidate treatments of the classification of anemia for the individual. In various examples, the computing system can determine that the individual is not following the one or more candidate treatments. In these scenarios, the computing system can send a first additional communication to a practitioner computing device indicating that the individual is not following the one or more recommended treatments and send a second additional communication to a patient computing device. The second additional communication can include a reminder for the individual to follow a recommended treatment for anemia.

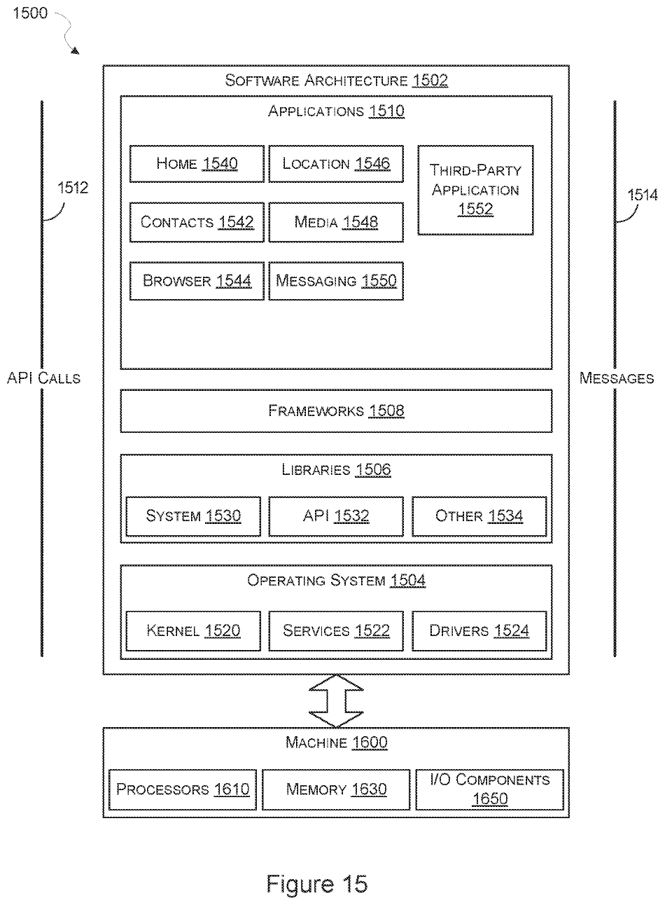

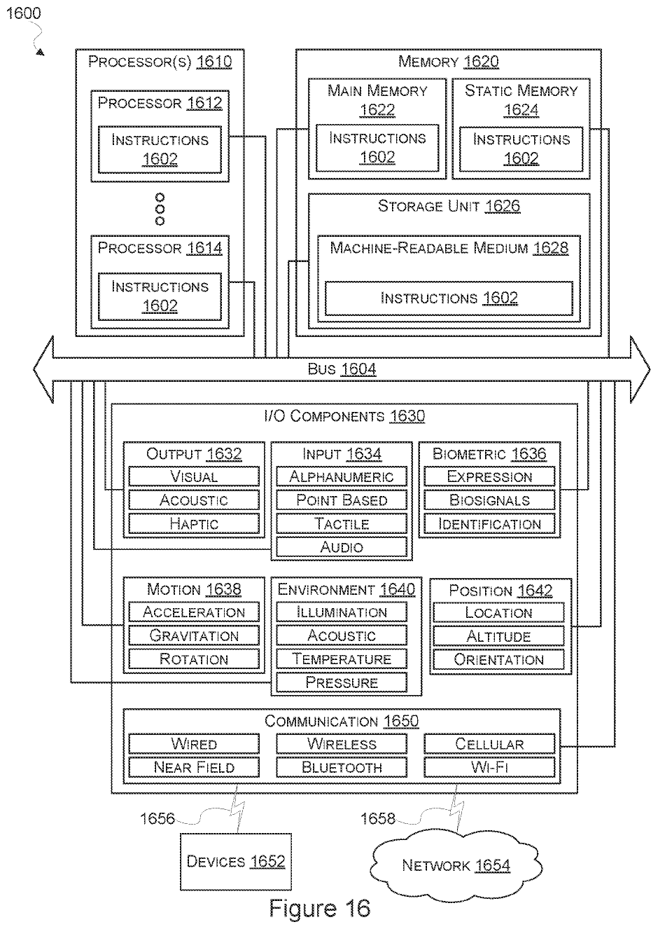

FIG. 15 is a block diagram 1500 illustrating an architecture for software 1502, which can be installed on any one or more of the devices described herein. FIG. 15 is merely a non-limiting example of a software architecture, and it will be appreciated that many other architectures can be implemented to facilitate the functionality described herein. In various embodiments, the software 1502 can be implemented by hardware such as a machine 1500 described in more detail with respect to FIG. 16 that includes processors 1610, memory 1620, and input/output (I/O) components 1630. In this example architecture, the software 1502 can be conceptualized as a stack of layers where each layer may provide a particular functionality. For example, the software 1502 can include layers such as an operating system 1504, libraries 1506, frameworks 1508, and applications 1510. Operationally, the applications 1510 invoke API calls 1512 through the software stack and receive messages 1514 in response to the API calls 1512, consistent with some implementations.

In various implementations, the operating system 1504 manages hardware resources and provides common services. The operating system 1504 can include, for example, a kernel 1520, services 1522, and drivers 1524. The kernel 1520 can act as an abstraction layer between the hardware and the other software layers, consistent with some embodiments. For example, the kernel 1520 can provide memory management, processor management (e.g., scheduling), component management, networking, and security settings, among other functionalities. The services 1522 can provide other common services for the other software layers. The drivers 1524 can be responsible for controlling or interfacing with the underlying hardware, according to some embodiments. For instance, the drivers 1524 can include display drivers, camera drivers, BLUETOOTH® or BLUETOOTH® Low-Energy drivers, flash memory drivers, serial communication drivers (e.g., Universal Serial Bus (USB) drivers), Wi-Fi® drivers, audio drivers, power management drivers, and so forth.

In some embodiments, the libraries 1506 can provide a low-level common infrastructure utilized by the applications 1510. The libraries 1506 can include system libraries 1530 (e.g., C standard library) that can provide functions such as memory allocation functions, string manipulation functions, mathematic functions, and the like. In addition, the libraries 1506 can include API libraries 1532 such as media libraries (e.g., libraries to support presentation and manipulation of various media formats such as Moving Picture Experts Group-4 (MPEG4), Advanced Video Coding (H.264 or AVC), Moving Picture Experts Group Layer-3 (MP3), Advanced Audio Coding (AAC), Adaptive Multi-Rate (AMR) audio codec, Joint Photographic Experts Group (JPEG or JPG), or Portable Network Graphics (PNG)), graphics libraries (e.g., an OpenGL framework used to render in 2D and 3D in a graphic context on a display), database libraries (e.g., SQLite to provide various relational database functions), web libraries (e.g., WebKit to provide web browsing functionality), and the like. The libraries 1506 can also include a wide variety of other libraries 1534 to provide many other APIs to the applications 1510.

The frameworks 1508 can provide a high-level common infrastructure that can be utilized by the applications 1510, according to some implementations. For example, the frameworks 1508 can provide various graphical user interface (GUI) functions, high-level resource management, high-level location services, and so forth. The frameworks 1508 can provide a broad spectrum of other APIs that can be utilized by the applications 1510, some of which may be specific to a particular operating system 1504 or platform.

In an example implementation, the applications 1510 can include a home application 1540, a contacts application 1542, a browser application 1544, a location application 1546, a media application 1548, a messaging application 1550, and a broad assortment of other applications, such as a third-party application 1552. According to some implementations, the applications 1510 can be programs that execute functions defined in the programs. Various programming languages can be employed to create one or more of the applications 1510, structured in a variety of manners, such as object-oriented programming languages (e.g., Objective-C, Java, or C++) or procedural programming languages (e.g., C or assembly language). In a specific example, the third-party application 1552 (e.g., an application developed using the ANDROID™ or IOS™ software development kit (SDK) by an entity other than the vendor of the particular platform) may be mobile software running on a mobile operating system such as IOS™, ANDROID™, WINDOWS® Phone, or another mobile operating system. In this example, the third-party application 1552 can invoke the API calls 1512 provided by the operating system 1504 to facilitate functionality described herein.

FIG. 14 illustrates a diagrammatic representation of a machine 1600 in the form of a computer system within which a set of instructions 1602 may be executed for causing the machine 1600 to perform any one or more of the methodologies discussed herein, according to one or more example implementations. Specifically, FIG. 14 shows a diagrammatic representation of the machine 1600 in the example form of a computer system, within which instructions (e.g., software, a program, an application, an applet, an app, or other executable code) for causing the machine 1600 to perform any one or more of the methodologies discussed herein may be executed. For example, the instructions 1602 can cause the machine 1600 to execute the processes and/or operations described with respect to FIG. 1 to FIG. 14. The instructions 1602 transform the general, non-programmed machine 1600 into a particular machine 1600 programmed to carry out the described and illustrated functions in the manner described. In alternative implementations, the machine 1600 operates as a standalone device or may be coupled (e.g., networked) to other machines. In a networked deployment, the machine 1600 may operate in the capacity of a server machine or a client machine in a server-client network environment, or as a peer machine in a peer-to-peer (or distributed) network environment. The machine 1600 can comprise, but not be limited to, a server computer, a client computer, a personal computer (PC), a tablet computer, a laptop computer, a netbook, a set-top box (STB), a personal digital assistant (PDA), an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 1602, sequentially or otherwise, that specify actions to be taken by the machine 1600. Further, while only a single machine 1600 is illustrated, the term “machine” shall also be taken to include a collection of machines 1600 that individually or jointly execute the instructions 1602 to perform any one or more of the methodologies discussed herein.

The machine 1600 can include processors 1610, memory 1620, and I/O components 1630, which may be configured to communicate with each other such as via a bus 1604. In an example embodiment, the processors 1610 (e.g., a central processing unit (CPU), a reduced instruction set computing (RISC) processor, a complex instruction set computing (CISC) processor, a graphics processing unit (GPU), a digital signal processor (DSP), an application-specific integrated circuit (ASIC), a radio-frequency integrated circuit (RFIC), another processor, or any suitable combination thereof) can include, for example, a processor 1612 and a processor 1614 that can execute the instructions 1602. The term “processor” is intended to include multi-core processors that may comprise two or more independent processors (sometimes referred to as “cores”) that may execute instructions 1602 contemporaneously. Although FIG. 16 shows multiple processors 1610, the machine 1600 may include a single processor 1612 with a single core, a single processor 1612 with multiple cores (e.g., a multi-core processor 1612), multiple processors 1612, 1614 with a single core, multiple processors 1612, 1614 with multiple cores, or any combination thereof.

The memory 1620 can include a main memory 1622, a static memory 1624, and a storage unit 1626, each accessible to the processors 1610 such as via the bus 1604. The storage unit 1626 can include at least one machine-readable medium 1628. The main memory 1622, the static memory 1624, the storage unit 1626, and/or the machine-readable medium 1628 can store the instructions 1602 embodying any one or more of the methodologies or functions described herein. The instructions 1602 can also reside, completely or partially, within the main memory 1622, within the static memory 1624, within the storage unit 1626, within the machine-readable medium 1628, within at least one of the processors 1610 (e.g., within the processor's cache memory), or any suitable combination thereof, during execution thereof by the machine 1600.

The I/O components 1630 can include a wide variety of components to receive input, provide output, produce output, transmit information, exchange information, capture measurements, and so on. The specific I/O components 1630 that are included in a particular machine 1600 can depend on the type of machine. For example, portable machines such as mobile phones can include a touch input device or other such input mechanisms, while a headless server machine will likely not include such a touch input device. It will be appreciated that the I/O components 1630 can include many other components that are not shown in FIG. 16. The I/O components 1630 can be grouped according to functionality merely for simplifying the following discussion, and the grouping is in no way limiting. In various example implementations, the I/O components 1630 can include output components 1632 and input components 1634. The output components 1632 can include visual components (e.g., a display such as a plasma display panel (PDP), a light-emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 1634 can include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example implementations, the I/O components 1630 can include biometric components 1636, motion components 1638, environmental components 1640, and/or position components 1642, among a wide array of other components. For example, the biometric components 1636 can include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 1638 can include acceleration sensor components (e.g., accelerometer), gravitation sensor components, rotation sensor components (e.g., gyroscope), and so forth. The environmental components 1640 can include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detect concentrations of hazardous gases for safety or to measure pollutants in the atmosphere), or other components that can provide indications, measurements, or signals corresponding to a surrounding physical environment. The position components 1642 can include location sensor components (e.g., a Global Positioning System (GPS) receiver component), altitude sensor components (e.g., altimeters or barometers that detect air pressure from which altitude may be derived), orientation sensor components (e.g., magnetometers), and the like.

Communication can be implemented using a wide variety of technologies. The I/O components 1630 can include communication components 1650 operable to couple the machine 1600 to a network 1654 or devices 1652 via a coupling 1656 and a coupling 1658, respectively. For example, the communication components 1650 can include a network interface component or another suitable device to interface with the network 1654. In further examples, the communication components 1650 can include wired communication components, wireless communication components, cellular communication components, near field communication (NFC) components, Bluetooth® components (e.g., Bluetooth® Low Energy), Wi-Fi® components, and other communication components to provide communication via other modalities. The devices 1652 can be another machine or any of a wide variety of peripheral devices (e.g., coupled via a USB).

Moreover, the communication components 1650 can detect identifiers or include components operable to detect identifiers. For example, the communication components 1650 can include radio-frequency identification (RFID) tag reader components, NFC smart tag detection components, optical reader components (e.g., an optical sensor to detect one-dimensional bar codes such as Universal Product Code (UPC) bar code, multi-dimensional bar codes such as QR code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information can be derived via the communication components 1650, such as location via Internet Protocol (IP) geolocation, location via Wi-Fi® signal triangulation, location via detecting an NFC beacon signal that may indicate a particular location, and so forth.

The various memories (i.e., 1620, 1622, 1624, and/or memory of the processor(s) 1610) and/or the storage unit 1626 can store one or more sets of instructions 1602 and data structures (e.g., software) embodying or utilized by any one or more of the methodologies or functions described herein. These instructions (e.g., the instructions 1602), when executed by the processor(s) 1610, cause various operations to implement the disclosed implementations.

As used herein, the terms “machine-storage medium,” “device-storage medium,” and “computer-storage medium” mean the same thing and can be used interchangeably. The terms refer to a single or multiple storage devices and/or media (e.g., a centralized or distributed database, and/or associated caches and servers) that store executable instructions 1602 and/or data. The terms shall accordingly be taken to include, but not be limited to, solid-state memories, and optical and magnetic media, including memory internal or external to processors 1610. Specific examples of machine-storage media, computer-storage media, and/or device-storage media include non-volatile memory, including by way of example semiconductor memory devices, e.g., erasable programmable read-only memory (EPROM), electrically erasable programmable read-only memory (EEPROM), field-programmable gate array (FPGA), and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

In various example embodiments, one or more portions of the network 1654 can be an ad hoc network, an intranet, an extranet, a virtual private network (VPN), a local-area network (LAN), a wireless LAN (WLAN), a wide-area network (WAN), a wireless WAN (WWAN), a metropolitan-area network (MAN), the Internet, a portion of the Internet, a portion of the public switched telephone network (PSTN), a plain old telephone service (POTS) network, a cellular telephone network, a wireless network, a Wi-Fi® network, another type of network, or a combination of two or more such networks. For example, the network 1654 or a portion of the network 1654 can include a wireless or cellular network, and the coupling 1658 can be a Code Division Multiple Access (CDMA) connection, a Global System for Mobile communications (GSM) connection, or another type of cellular or wireless coupling. In this example, the coupling 1656 can implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High-Speed Packet Access (HSPA), Worldwide Interoperability for Microwave Access (WiMAX), Long-Term Evolution (LTE) standard, others defined by various standard-setting organizations, other long-range protocols, or other data transfer technology.

The instructions 1602 can be transmitted or received over the network 1654 using a transmission medium via a network interface device (e.g., a network interface component included in the communication components 1650) and utilizing any one of a number of well-known transfer protocols (e.g., Hypertext Transfer Protocol (HTTP)). Similarly, the instructions 1602 can be transmitted or received using a transmission medium via the coupling 1658 (e.g., a peer-to-peer coupling) to the devices 1652. The terms “transmission medium” and “signal medium” mean the same thing and can be used interchangeably in this disclosure. The terms “transmission medium” and “signal medium” shall be taken to include any intangible medium that is capable of storing, encoding, or carrying the instructions 1602 for execution by the machine 1600, and include digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a manner as to encode information in the signal.

The terms “machine-readable medium,” “computer-readable medium,” and “device-readable medium” mean the same thing and may be used interchangeably in this disclosure. The terms are defined to include both machine-storage media and transmission media. Thus, the terms include both storage devices/media and carrier waves/modulated data signals.

Unless otherwise indicated, all numbers expressing quantities of physical properties, chemical properties, dimensions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the implementations described herein. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±100/0 of the stated value; ±9% of the stated value; ±8% of the stated value; ±7% of the stated value; ±6% of the stated value; ±5% of the stated value; ±4% of the stated value; ±3% of the stated value; ±2% of the stated value, or ±1% of the stated value.

### Illustrative Examples

Example 1. An apparatus comprising: a casing that defines a lumen, the lumen being accessible via an opening in the casing, the opening having a width from about 0.1 cm to about 4 cm and the casing having a height from about 0.5 cm to about 10 cm; and a plurality of electronic components disposed within the casing, the plurality of electronic components including: emitter circuitry that emits an amount of electromagnetic radiation having a plurality of wavelength ranges, each wavelength range of the plurality of wavelength ranges comprising a different subset of wavelengths from about 300 nm to about 1500 nm; and detector circuitry that detects at least a portion of the amount of electromagnetic radiation emitted by the emitter circuitry.

Example 2. The apparatus of example 1, wherein individual electronic components of the plurality of electronic components are disposed radially around at least a portion of the lumen.

Example 3. The apparatus of example 1 or 2, comprising a plurality of visual indicators, individual visual indicators of the plurality of visual indicators corresponding to a respective color.

Example 4. The apparatus of example 3, wherein: individual visual indicators of the plurality of visual indicators are disposed radially around at least a portion of the lumen; the plurality of visual indicators includes a first indicator corresponding to a first color, a second indicator corresponding to a second color, and a third indicator corresponding to a third color; and the first color corresponds to a first number of probabilities of an individual being diagnosed with anemia, the second color corresponds to a second number of probabilities of the individual being diagnosed with anemia, and the third color corresponds to a third number of probabilities of the individual being diagnosed with anemia.

Example 5. The apparatus of any of examples 1-4, comprising: processor circuitry; and one or more non-transitory data storage devices storing computer-readable instructions that, when executed by the processor circuitry, cause the processor circuitry to: activate the emitter circuitry to emit electromagnetic radiation having one or more wavelengths within a subset of the plurality of wavelength ranges for a period of time, the electromagnetic radiation having one or more first intensity values; activate the detector circuitry during the period of time; and determine an amount of the electromagnetic radiation detected by the detector circuitry during the period of time, the amount of the electromagnetic radiation having one or more second intensity values.

Example 6. The apparatus of example 5, wherein the one or more non-transitory data storage devices store additional computer-readable instructions that, when executed by the processor circuitry, cause the processor circuitry to: determine one or more amounts of difference between the one or more first intensity values and the one or more second intensity values; determine an amount of attenuation of the amount of electromagnetic radiation based on the one or more amounts of difference; determine, based on the amount of attenuation, an amount of a hemoglobin metalloprotein in blood of an individual; and determine a probability of anemia being present in the individual based on the amount of the hemoglobin metalloprotein.

Example 7. The apparatus of example 6, wherein the one or more non-transitory data storage devices store further computer-readable instructions that, when executed by the processor circuitry, cause the processor circuitry to activate an indicator of a plurality of indicators that corresponds to the probability of anemia being present in the individual.

Example 8. The apparatus of any one of examples 5-7, wherein the one or more non-transitory data storage devices store additional computer-readable instructions that, when executed by the processor circuitry, cause the processor circuitry to activate the emitter circuitry to emit the amount of electromagnetic radiation based on an activation input.

Example 9. The apparatus of example 8, comprising an input device coupled to one or more electronic components of the plurality of electronic components, the input device to receive the activation input.

Example 10. The apparatus of any one of example 1-9, wherein the emitter circuitry includes a plurality of light emitting diodes (LEDs) and the detector circuitry includes one or more photodiodes.

Example 11. The apparatus of example 10, wherein the plurality of LEDs includes a first LED that emits electromagnetic radiation having first wavelengths from about 540 nm to about 690 nm, a second LED that emits electromagnetic radiation having second wavelengths from about 710 to about 770 nm, a third LED that emits electromagnetic radiation having third wavelengths from about 780 nm to about 840 nm, a fourth LED that emits electromagnetic radiation having wavelengths from about 810 nm to about 900 nm, and a fifth LED that emits electromagnetic radiation having fourth wavelengths from about 910 nm to about 990 nm.

Example 12. The apparatus of any one of examples 1-11, comprising: one or more energy storage devices; power circuitry to control a supply of power from the one or more energy storage devices to the plurality of electronic components; and communications circuitry to transmit and receive signals according to one or more communication protocols.

Example 13. The apparatus of example 12, wherein the one or more energy storage devices include at least one of a battery or a supercapacitor and the communications circuitry transmits a plurality of signals to a computing device according to a wireless local area network communications protocol, the plurality of signals including first data that corresponds to first electromagnetic radiation emitted by the emitter circuitry and second data that corresponds to second electromagnetic radiation detected by the detector circuitry.

Example 14. The apparatus of any one of examples 1-13, wherein: the casing is comprised of a polymeric material; the casing has a first width from about 1 cm to about 5 cm; the lumen has a second width that is less than the first width, the second width being from about 0.8 cm to about 4.5 cm; and having a height from about 4 cm to about 6.4 cm.

Example 15. The apparatus of any one of examples 1-13, wherein the casing is comprised of a fabric material.

Example 16. The apparatus of any one of examples 1-15, comprising signal conditioning circuitry to filter additional electromagnetic radiation corresponding to ambient light from an environment that includes the apparatus, the additional electromagnetic radiation being detected by the detector circuitry.

Example 17. A method comprising: activating a plurality of emitters of a wearable device to emit electromagnetic radiation having a plurality of wavelength ranges, each wavelength range of the plurality of wavelength ranges comprising a different subset of wavelengths from about 300 nm to about 1500 nm; activating one or more detectors of the wearable device to detect an amount of the electromagnetic radiation emitted by the plurality of emitters; determining, by the wearable device, one or more differences between first intensities of the electromagnetic radiation emitted by the plurality of emitters and second intensities of the electromagnetic radiation detected by the one or more detectors; determining, by the wearable device based on the one or more differences, an amount of a hemoglobin metalloprotein in blood of an individual; determining, by the wearable device, a probability of anemia being present in the individual according to the amount of the hemoglobin metalloprotein in the blood of the individual; determining, by the wearable device, an indicator of a plurality of indicators that are included in the wearable device, the indicator corresponding to the probability of anemia being present in the individual; and activating the indicator.

Example 18. The method of example 11, wherein activating the plurality of emitters and activating the one or more detectors is part of a protocol to determine a probability of anemia being present in individuals; wherein the protocol includes emitting first electromagnetic radiation having a first range of wavelengths for a first period of time, emitting second electromagnetic radiation having a second range of wavelengths for a second period of time subsequent to the first period of time, and emitting third electromagnetic radiation having a third range of wavelengths for a third period of time subsequent to the second period of time.

Example 19. The method of example 18, wherein a first pause takes place between the first period of time and the second period of time and a second pause takes place between the second period of time and the third period of time.

Example 20. The method of any one of examples 17-19, comprising sending the probability of anemia being present in the individual to a computing device via a wireless communication interface of the wearable device.

Example 21. The method of any one of example 17-20, comprising: sending emission data to a computing device via a wireless communication interface of the wearable device, the emission data corresponding to electromagnetic radiation emitted by the wearable device over a period of time, wherein the emission data indicates a range of wavelengths of the electromagnetic radiation, the period of time, and an intensity of the electromagnetic radiation emitted by the plurality of emitters; and sending detection data to the computing device via the wireless communication interface, the detection data corresponding to an amount of the electromagnetic radiation detected by the one or more detectors, the detection data indicating the range of wavelengths of the electromagnetic radiation, the period of time, and an additional intensity of the amount of the electromagnetic radiation detected by the one or more detectors.

Example 22. The method of example 21, wherein the computing device is a mobile computing device executing an application that displays at least one of the probabilities of anemia being present in the individual or the amount of the hemoglobin metalloprotein in the blood of the individual.

Example 23. The method of example 21 or 22, wherein the computing device is associated with a healthcare practitioner related to the individual.

Example 24. The method of any one of examples 17-22, comprising receiving input via an input device of the wearable device to activate the plurality of emitters.

Example 25. The method of any one of examples 17-24, comprising: emitting, by one or more first emitters of the plurality of emitters, first electromagnetic radiation having a first wavelength range of the plurality of wavelength ranges, the first electromagnetic radiation having a first measure of intensity; and determining an amount of the first electromagnetic radiation detected by the one or more detectors, the amount of the first electromagnetic radiation detected by the one or more detectors having a second measure of intensity; wherein the amount of the hemoglobin metalloprotein in the blood of the individual is determined based on a difference between the first measure of intensity and the second measure of intensity.

Example 26. The method of example 25, comprising: emitting, by one or more second emitters of the plurality of emitters, second electromagnetic radiation having a second wavelength range of the plurality of wavelength ranges, the second electromagnetic radiation having a third measure of intensity and the second wavelength range being different from the first wavelength range; determining an amount of the second electromagnetic radiation detected by the one or more detectors, the amount of the second electromagnetic radiation detected by the one or more detectors having a fourth measure of intensity; determining a difference between the third measure of intensity and the fourth measure of intensity; and determining a peripheral capillary oxygen saturation (SpO₂) level of the individual based on the difference between the third measure of intensity and the fourth measure of intensity.

Example 27. The method of example 26, comprising: emitting, by one or more third emitters of the plurality of emitters, third electromagnetic radiation having a third wavelength range of the plurality of wavelength ranges, the third electromagnetic radiation having a fifth measure of intensity and the third wavelength range being different from the first wavelength range and the second wavelength range; determining an amount of the third electromagnetic radiation detected by the one or more detectors, the amount of the third electromagnetic radiation detected by the one or more detectors having a sixth measure of intensity; determining a difference between the fifth measure of intensity and the sixth measure of intensity; and determining a packed cell volume of the blood of the individual based on the difference between the fifth measure of intensity and the sixth measure of intensity; wherein the probability of anemia being present in the individual is based on the packed cell volume.

Example 28. A computing system comprising: one or more hardware processors; and one or more memory devices, the one or more memory devices storing computer-readable instructions that, when executed by the one or more hardware processors, cause the one or more hardware processors to perform operations comprising: obtaining sensor data from a data capture device, the sensor data including first intensity values of emitted electromagnetic radiation and second intensity values of detected electromagnetic radiation; determining differences between at least a portion of the first intensity values and at least a portion of the second intensity values; determining, based on the differences, one or more amounts of attenuation of the emitted electromagnetic radiation; determining, based on the one or more amounts of attenuation, a quantity of hemoglobin metalloproteins included in blood of an individual; performing an analysis of the quantity of hemoglobin metalloproteins in relation to one or more threshold quantities of hemoglobin metalloproteins, individual threshold quantities of hemoglobin metalloproteins corresponding to a range of probabilities corresponding to an anemia diagnosis; determining, based on the analysis, one or more probabilities of anemia being present in the individual; determining, based on the quantity of hemoglobin metalloproteins included in the blood of the individual, a classification of anemia for the individual; determining, based on the one or more probabilities of anemia being present in the individual and on the classification of anemia for the individual, one or more candidate treatments of the classification of anemia for the individual; and sending a communication to a computing device of the individual, the communication indicating at least one of the one or more probabilities of anemia being present in the individual, the classification of anemia for the individual, or the one or more candidate treatments of the classification of anemia for the individual.

Example 29. The computing system of example 28, comprising additional computer-readable instructions that, when executable by the one or more hardware processors, cause the one or more hardware processors to perform additional operations comprising: determining, based on the sensor data, first differences between first intensities of first emitted electromagnetic radiation and second intensities of second detected electromagnetic radiation, the first emitted electromagnetic radiation and the second detected electromagnetic radiation having a first range of wavelengths; determining, based on the first differences, a first amount of attenuation of the first emitted electromagnetic radiation; and determining, based on the first amount of attenuation, an amount of oxygenated hemoglobin included in blood of the individual.

Example 30. The computing system of example 29, comprising additional computer-readable instructions that, when executable by the one or more hardware processors, cause the one or more hardware processors to perform additional operations comprising: determining, based on the sensor data, second differences between third intensities of third emitted electromagnetic radiation and fourth intensities of fourth detected electromagnetic radiation, the third emitted electromagnetic radiation and the fourth detected electromagnetic radiation having a second range of wavelengths; determining, based on the second differences, a second amount of attenuation of the third emitted electromagnetic radiation; and determining, based on the second amount of attenuation, an amount of deoxygenated hemoglobin included in blood of the individual.

Example 31. The computing system of example 30, comprising additional computer-readable instructions that, when executable by the one or more hardware processors, cause the one or more hardware processors to perform additional operations comprising: determining a measure of a total amount of hemoglobin included in the blood of the individual based on the amount of oxygenated hemoglobin in the blood of the individual and the amount of deoxygenated hemoglobin in the blood of the individual, wherein the one or more probabilities of anemia being present in the individual are based on the measure of the total amount of hemoglobin included in the blood of the individual.

Example 32. The computing system of example 30, wherein the first range of wavelengths is from about 600 nm to about 770 nm and the second range of wavelengths is from about 820 nm to about 990 nm.

Example 33. The computing system of example 30, comprising additional computer-readable instructions that, when executable by the one or more hardware processors, cause the one or more hardware processors to perform additional operations comprising: determining, based on at least one of the amount of oxygenated hemoglobin in the blood of the individual or the amount of deoxygenated hemoglobin in the blood of the individual, a level of hematocrit, and wherein the one or more probabilities of anemia being present in the individual are based on the level of hematocrit.

Example 34. The computing system of example 28, comprising additional computer-readable instructions that, when executable by the one or more hardware processors, cause the one or more hardware processors to perform additional operations comprising: determining one or more procedures to recommend for the individual based on at least one of the one or more probabilities of anemia being present in the individual or the classification of anemia of the individual.

Example 35. The computing system of example 28, comprising additional computer-readable instructions that, when executable by the one or more hardware processors, cause the one or more hardware processors to perform additional operations comprising: determining that the individual is not following at least one candidate treatment of the one or more candidate treatments; sending a first additional communication to a practitioner computing device indicating that the individual is not following the at least one candidate treatment of the one or more candidate treatments; and sending a second additional communication to a patient computing device, the communication including a reminder for the individual to follow the at least one candidate treatment of the one or more candidate treatments.

Example 36. The computing system of example 28, comprising additional computer-readable instructions that, when executable by the one or more hardware processors, cause the one or more hardware processors to perform additional operations comprising: performing an analysis of a portion of a corpus of data that indicates a plurality of biological characteristics of individuals that have been diagnosed with anemia; determining, based on the analysis, a plurality of measures of statistical correlations between groups of biological characteristics included in the plurality of biological characteristics; and determining, based on the plurality of measures of statistical correlations, at least one of one or more variables, one or more nodes, or one or more weights of a computational model to determine probabilities of individuals being diagnosed with anemia.

Example 37. The computing system of example 36, comprising additional computer-readable instructions that, when executable by the one or more hardware processors, cause the one or more hardware processors to perform additional operations comprising: determining, based on the sensor data, a feature of the individual that corresponds to at least one of a variable of the one or more variables or a node of the one or more nodes of the computational model; determining a measure of the feature of the individual; and implementing the computational model using the measure of the feature to determine the probabilities of the individual being diagnosed with anemia.

Example 38. The computing system of example 37, wherein the feature includes oxygenated hemoglobin levels of the individual and the computing system includes additional computer-readable instructions that, when executable by the one or more hardware processors, cause the one or more hardware processors to perform additional operations comprising: determining, based on the sensor data, an amount of attenuation of a range of wavelengths of electromagnetic radiation emitted by the data capture device; determining a measure oxygenated hemoglobin levels of the individual based on the amount of attenuation of the range of wavelengths of electromagnetic radiation emitted by the data capture device; and implementing the computational model using the measure of oxygenated hemoglobin levels of the individual to determine the one or more probabilities of anemia being present in the individual.

Example 39. The computing system of example 36, wherein the corpus of data includes a plurality of treatments of anemia and the computing system includes additional computer-readable instructions that, when executable by the one or more hardware processors, cause the one or more hardware processors to perform additional operations comprising: performing an additional analysis of an additional portion of the corpus of data to determine a plurality of measures of additional correlations between one or more biological characteristics of the plurality of biological characteristics and one or more treatments of the plurality of treatments; and determining, based on the plurality of measures of additional correlations, at least one of one or more additional variables, one or more additional nodes, or one or more additional weights of an additional computational model to determine at least one recommendation for treatment of individuals diagnosed with. anemia.

Example 40. The computing system of example 28, wherein the sensor data is obtained from one or more data capture devices.

Example 41. The computing system of example 28, wherein the sensor data is obtained from a computing device that executes an application to collect the sensor data from one or more data capture devices and to send the sensor data to the computing system.

Example 42. The computing system of example 28, comprising additional computer-readable instructions that, when executable by the one or more hardware processors, cause the one or more hardware processors to perform additional operations comprising: obtaining authentication data from at least one of a computing device or a data capture device; determining that the authentication data corresponds to at least one of an individual or a device that is granted access to exchange information with the computing system; registering at least one of the computing device or the data capture device with the computing system; and storing the sensor data in one or more data stores in electronic data stores in electronic communication with the computing system, wherein the sensor data is stored in the one or more data stores in association with one or more identifiers of at least one of the individual or the device.

Example 43. An apparatus comprising: a body having a length from about 4 cm to about 10 cm and a width from about 1 cm to about 4 cm; a clamp member coupled to the body, the clamp member having a concave shape; and a plurality of electronic components disposed within the body, the plurality of electronic components including: emitter circuitry that emits an amount of electromagnetic radiation having a plurality of wavelength ranges, each wavelength range of the plurality of wavelength ranges comprising a different subset of wavelengths from about 300 nm to about 1500 nm; and detector circuitry that detects at least a portion of the amount of electromagnetic radiation emitted by the emitter circuitry.

Example 44. The apparatus of example 43, comprising a plurality of visual indicators, individual visual indicators of the plurality of visual indicators corresponding to a respective color.

Example 45. The apparatus of example 44, wherein: the plurality of visual indicators includes a first indicator corresponding to a first color and a second indicator corresponding to a second color, and the first color corresponds to a first number of probabilities of an individual being diagnosed with anemia and the second color corresponds to a second number of probabilities of the individual being diagnosed with anemia.

Example 46. The apparatus of any of examples 43-45, comprising: processor circuitry; and one or more non-transitory data storage devices storing computer-readable instructions that, when executed by the processor circuitry, cause the processor circuitry to: activate the emitter circuitry to emit electromagnetic radiation having one or more wavelengths within a subset of the plurality of wavelength ranges for a period of time, the electromagnetic radiation having one or more first intensity values; activate the detector circuitry during the period of time; and determine an amount of the electromagnetic radiation detected by the detector circuitry during the period of time, the amount of the electromagnetic radiation having one or more second intensity values.

Example 47. The apparatus of example 46, wherein the one or more non-transitory data storage devices store additional computer-readable instructions that, when executed by the processor circuitry, cause the processor circuitry to: determine one or more amounts of difference between the one or more first intensity values and the one or more second intensity values; determine an amount of attenuation of the first electromagnetic radiation based on the one or more amounts of difference; determine, based on the amount of attenuation, an amount of a hemoglobin metalloprotein in blood of an individual; and determine a probability of anemia being present in the individual based on the amount of the hemoglobin metalloprotein.

Example 48. The apparatus of example 47, wherein the one or more non-transitory data storage devices store further computer-readable instructions that, when executed by the processor circuitry, cause the processor circuitry to activate an indicator of a plurality of indicators that corresponds to the probability of anemia being present in the individual.

Example 49. The apparatus of any one of examples 46-48, wherein the one or more non-transitory data storage devices store additional computer-readable instructions that, when executed by the processor circuitry, cause the processor circuitry to activate the emitter circuitry to emit the amount of electromagnetic radiation based on an activation input.

Example 50. The apparatus of example 49, comprising an input device coupled to one or more electronic components of the plurality of electronic components, the input device to receive the activation input.

Example 51. The apparatus of any one of examples 43-50, wherein the emitter circuitry includes a plurality of light emitting diodes (LEDs) and the detector circuitry includes one or more photodiodes.

Example 52. The apparatus of example 51, wherein the plurality of LEDs includes a first LED that emits electromagnetic radiation having first wavelengths from about 610 nm to about 690 nm, a second LED that emits electromagnetic radiation having second wavelengths from about 710 to about 770 nm, a third LED that emits electromagnetic radiation having third wavelengths from about 780 nm to about 840 nm, a fourth LED that emits electromagnetic radiation from about 810 nm to about 900 nm, and a fifth LED that emits electromagnetic radiation having fourth wavelengths from about 910 nm to about 990 nm.

Example 53. The apparatus of any one of examples 43-52, comprising: one or more energy storage devices; power circuitry to control a supply of power from the one or more energy storage devices to the plurality of electronic components; and communications circuitry to transmit and receive signals according to one or more communication protocols.

Example 54. The apparatus of example 53, wherein the one or more energy storage devices include at least one of a battery or a supercapacitor and the communications circuitry transmits a plurality of signals to a computing device according to a wireless local area network communications protocol, the plurality of signals including first data that corresponds to first electromagnetic radiation emitted by the emitter circuitry and second data that corresponds to second electromagnetic radiation detected by the detector circuitry.

Example 55. The apparatus of any one of examples 43-54, wherein the clamp member is configured to attach to a finger of an individual.

Example 56. The apparatus of any one of examples 43-55, wherein the body and the clamp member are comprised of a polymeric material.

Example 57. The apparatus of any one of examples 43-56, comprising signal conditioning circuitry to filter additional electromagnetic radiation corresponding to ambient light from an environment that includes the apparatus, the additional electromagnetic radiation being detected by the detector circuitry.

1-16. (canceled)

17. A method comprising:
activating a plurality of emitters of a wearable device to emit electromagnetic radiation having a plurality of wavelength ranges, each wavelength range of the plurality of wavelength ranges comprising a different subset of wavelengths from about 300 nm to about 1500 nm;
activating one or more detectors of the wearable device to detect an amount of the electromagnetic radiation emitted by the plurality of emitters;
determining, by the wearable device, one or more differences between first intensities of the electromagnetic radiation emitted by the plurality of emitters and second intensities of the electromagnetic radiation detected by the one or more detectors;
determining, by the wearable device based on the one or more differences, an amount of a hemoglobin metalloprotein in blood of an individual;
determining, by the wearable device, a probability of anemia being present in the individual according to the amount of the hemoglobin metalloprotein in the blood of the individual;
determining, by the wearable device, an indicator of a plurality of indicators that are included in the wearable device, the indicator corresponding to the probability of anemia being present in the individual; and
activating the indicator.

18. The method of claim 17, wherein activating the plurality of emitters and activating the one or more detectors is part of a protocol to determine a probability of anemia being present in individuals; wherein the protocol includes emitting first electromagnetic radiation having a first range of wavelengths for a first period of time, emitting second electromagnetic radiation having a second range of wavelengths for a second period of time subsequent to the first period of time, and emitting third electromagnetic radiation having a third range of wavelengths for a third period of time subsequent to the second period of time.

19. The method of claim 18, wherein a first pause takes place between the first period of time and the second period of time and a second pause takes place between the second period of time and the third period of time.

20. (canceled)

21. The method of claim 17, comprising:
sending emission data to a computing device via a wireless communication interface of the wearable device, the emission data corresponding to electromagnetic radiation emitted by the wearable device over a period of time, wherein the emission data indicates a range of wavelengths of the electromagnetic radiation, the period of time, and an intensity of the electromagnetic radiation emitted by the plurality of emitters; and
sending detection data to the computing device via the wireless communication interface, the detection data corresponding to an amount of the electromagnetic radiation detected by the one or more detectors, the detection data indicating the range of wavelengths of the electromagnetic radiation, the period of time, and an additional intensity of the amount of the electromagnetic radiation detected by the one or more detectors.

22. The method of claim 21, wherein the computing device is a mobile computing device executing an application that displays at least one of the probability of anemia being present in the individual or the amount of the hemoglobin metalloprotein in the blood of the individual.

23. (canceled)

24. (canceled)

25. The method of claim 17, comprising:
emitting, by one or more first emitters of the plurality of emitters, first electromagnetic radiation having a first wavelength range of the plurality of wavelength ranges, the first electromagnetic radiation having a first measure of intensity; and
determining an amount of the first electromagnetic radiation detected by the one or more detectors, the amount of the first electromagnetic radiation detected by the one or more detectors having a second measure of intensity;
wherein the amount of the hemoglobin metalloprotein in the blood of the individual is determined based on a difference between the first measure of intensity and the second measure of intensity.

26. The method of claim 25, comprising:
emitting, by one or more second emitters of the plurality of emitters, second electromagnetic radiation having a second wavelength range of the plurality of wavelength ranges, the second electromagnetic radiation having a third measure of intensity and the second wavelength range being different from the first wavelength range;
determining an amount of the second electromagnetic radiation detected by the one or more detectors, the amount of the second electromagnetic radiation detected by the one or more detectors having a fourth measure of intensity;
determining a difference between the third measure of intensity and the fourth measure of intensity; and
determining a peripheral capillary oxygen saturation (SpO₂) level of the individual based on the difference between the third measure of intensity and the fourth measure of intensity.

27. The method of claim 26, comprising:
emitting, by one or more third emitters of the plurality of emitters, third electromagnetic radiation having a third wavelength range of the plurality of wavelength ranges, the third electromagnetic radiation having a fifth measure of intensity and the third wavelength range being different from the first wavelength range and the second wavelength range;
determining an amount of the third electromagnetic radiation detected by the one or more detectors, the amount of the third electromagnetic radiation detected by the one or more detectors having a sixth measure of intensity;
determining a difference between the fifth measure of intensity and the sixth measure of intensity; and
determining a packed cell volume of the blood of the individual based on the difference between the fifth measure of intensity and the sixth measure of intensity;
wherein the probability of anemia being present in the individual is based on the packed cell volume.

28. A computing system comprising:
one or more hardware processors; and
one or more memory devices, the one or more memory devices storing computer-readable instructions that, when executed by the one or more hardware processors, cause the one or more hardware processors to perform operations comprising:
obtaining sensor data from a data capture device, the sensor data including first intensity values of emitted electromagnetic radiation and second intensity values of detected electromagnetic radiation;
determining differences between at least a portion of the first intensity values and at least a portion of the second intensity values;
determining, based on the differences, one or more amounts of attenuation of the emitted electromagnetic radiation;
determining, based on the one or more amounts of attenuation, a quantity of hemoglobin metalloproteins included in blood of an individual;
performing an analysis of the quantity of hemoglobin metalloproteins in relation to one or more threshold quantities of hemoglobin metalloproteins, individual threshold quantities of hemoglobin metalloproteins corresponding to a range of probabilities corresponding to an anemia diagnosis;
determining, based on the analysis, one or more probabilities of anemia being present in the individual;
determining, based on the quantity of hemoglobin metalloproteins included in the blood of the individual, a classification of anemia for the individual;
determining, based on the one or more probabilities of anemia being present in the individual and on the classification of anemia for the individual, one or more candidate treatments of the classification of anemia for the individual; and
sending a communication to a computing device of the individual, the communication indicating at least one of the one or more probabilities of anemia being present in the individual, the classification of anemia for the individual, or the one or more candidate treatments of the classification of anemia for the individual.

29. The computing system of claim 28, comprising additional computer-readable instructions that, when executable by the one or more hardware processors, cause the one or more hardware processors to perform additional operations comprising:
determining, based on the sensor data, first differences between first intensities of first emitted electromagnetic radiation and second intensities of second detected electromagnetic radiation, the first emitted electromagnetic radiation and the second detected electromagnetic radiation having a first range of wavelengths;
determining, based on the first differences, a first amount of attenuation of the first emitted electromagnetic radiation; and
determining, based on the first amount of attenuation, an amount of oxygenated hemoglobin included in blood of the individual.

30. The computing system of claim 29, comprising additional computer-readable instructions that, when executable by the one or more hardware processors, cause the one or more hardware processors to perform additional operations comprising:
determining, based on the sensor data, second differences between third intensities of third emitted electromagnetic radiation and fourth intensities of fourth detected electromagnetic radiation, the third emitted electromagnetic radiation and the fourth detected electromagnetic radiation having a second range of wavelengths;
determining, based on the second differences, a second amount of attenuation of the third emitted electromagnetic radiation; and
determining, based on the second amount of attenuation, an amount of deoxygenated hemoglobin included in blood of the individual;
wherein the first range of wavelengths is from about 900 nm to about 770 nm and the second range of wavelengths is from about 1120 nm to about 990 nm.

31. The computing system of claim 30, comprising additional computer-readable instructions that, when executable by the one or more hardware processors, cause the one or more hardware processors to perform additional operations comprising:
determining a measure of a total amount of hemoglobin included in the blood of the individual based on the amount of oxygenated hemoglobin in the blood of the individual and the amount of deoxygenated hemoglobin in the blood of the individual, wherein the one or more probabilities of anemia being present in the individual are based on the measure of the total amount of hemoglobin included in the blood of the individual.

32. (canceled)

33. The computing system of claim 30, comprising additional computer-readable instructions that, when executable by the one or more hardware processors, cause the one or more hardware processors to perform additional operations comprising:
determining, based on at least one of the amount of oxygenated hemoglobin in the blood of the individual or the amount of deoxygenated hemoglobin in the blood of the individual, a level of hematocrit, and wherein the one or more probabilities of anemia being present in the individual are based on the level of hematocrit.

34. (canceled)

35. The computing system of claim 28, comprising additional computer-readable instructions that, when executable by the one or more hardware processors, cause the one or more hardware processors to perform additional operations comprising:
determining that the individual is not following at least one candidate treatment of the one or more candidate treatments;
sending a first additional communication to a practitioner computing device indicating that the individual is not following the at least one candidate treatment of the one or more candidate treatments; and
sending a second additional communication to a patient computing device, the communication including a reminder for the individual to follow the at least one candidate treatment of the one or more candidate treatments.

36. The computing system of claim 28, comprising additional computer-readable instructions that, when executable by the one or more hardware processors, cause the one or more hardware processors to perform additional operations comprising:
performing an analysis of a portion of a corpus of data that indicates a plurality of biological characteristics of individuals that have been diagnosed with anemia;
determining, based on the analysis, a plurality of measures of statistical correlations between groups of biological characteristics included in the plurality of biological characteristics; and
determining, based on the plurality of measures of statistical correlations, at least one of one or more variables, one or more nodes, or one or more weights of a computational model to determine probabilities of individuals being diagnosed with anemia.

37. The computing system of claim 36, comprising additional computer-readable instructions that, when executable by the one or more hardware processors, cause the one or more hardware processors to perform additional operations comprising:
determining, based on the sensor data, a feature of the individual that corresponds to at least one of a variable of the one or more variables or a node of the one or more nodes of the computational model;
determining a measure of the feature of the individual; and
implementing the computational model using the measure of the feature to determine the probabilities of the individual being diagnosed with anemia.

38. The computing system of claim 37, wherein the feature includes oxygenated hemoglobin levels of the individual and the computing system includes additional computer-readable instructions that, when executable by the one or more hardware processors, cause the one or more hardware processors to perform additional operations comprising:
determining, based on the sensor data, an amount of attenuation of a range of wavelengths of electromagnetic radiation emitted by the data capture device;
determining a measure oxygenated hemoglobin levels of the individual based on the amount of attenuation of the range of wavelengths of electromagnetic radiation emitted by the data capture device; and
implementing the computational model using the measure of oxygenated hemoglobin levels of the individual to determine the one or more probabilities of anemia being present in the individual.

39. The computing system of claim 36, wherein the corpus of data includes a plurality of treatments of anemia and the computing system includes additional computer-readable instructions that, when executable by the one or more hardware processors, cause the one or more hardware processors to perform additional operations comprising:
performing an additional analysis of an additional portion of the corpus of data to determine a plurality of measures of additional correlations between one or more biological characteristics of the plurality of biological characteristics and one or more treatments of the plurality of treatments; and
determining, based on the plurality of measures of additional correlations, at least one of one or more additional variables, one or more additional nodes, or one or more additional weights of an additional computational model to determine at least one recommendation for treatment of individuals diagnosed with. anemia

40-42. (canceled)

43. An apparatus comprising:
a body having a length from about 4 cm to about 10 cm and a width from about 1 cm to about 4 cm;
a clamp member coupled to the body, the clamp member having a concave shape;
a plurality of electronic components disposed within the body, the plurality of electronic components including:
emitter circuitry that emits an amount of electromagnetic radiation having a plurality of wavelength ranges, each wavelength range of the plurality of wavelength ranges comprising a different subset of wavelengths from about 300 nm to about 1500 nm; and
detector circuitry that detects at least a portion of the amount of electromagnetic radiation emitted by the emitter circuitry; and
a plurality of visual indicators that includes a first indicator corresponding to a first color and a second indicator corresponding to a second color, wherein the first color corresponds to a first number of probabilities of an individual being diagnosed with anemia and the second color corresponds to a second number of probabilities of the individual being diagnosed with anemia.

44. (canceled)

45. (canceled)

46. The apparatus of claim 43, comprising:
processor circuitry; and
one or more non-transitory data storage devices storing computer-readable instructions that, when executed by the processor circuitry, cause the processor circuitry to:
activate the emitter circuitry to emit electromagnetic radiation having one or more wavelengths within a subset of the plurality of wavelengths for a period of time, the electromagnetic radiation having one or more first intensity values;
activate the detector circuitry during the period of time;
determine an amount of the electromagnetic radiation detected by the detector circuitry during the period of time, the amount of the electromagnetic radiation having one or more second intensity values;
determine one or more amounts of difference between the one or more first intensity values and the one or more second intensity values;
determine an amount of attenuation of the first electromagnetic radiation based on the one or more amounts of difference;
determine, based on the amount of attenuation, an amount of a hemoglobin metalloprotein in blood of an individual; and
determine a probability of anemia being present in the individual based on the amount of the hemoglobin metalloprotein.

47.-57. (canceled)
